(19) United States
(12) Patent Application Publication
(10) Pub. No.: US 2023/0040415 A1
(43) Pub. Date: Feb. 9, 2023

(54) METHODS OF TREATING LYMPHEDEMA WITH DEUPIRFENIDONE

(71) Applicant: PureTech LYT 100, Inc., Boston, MA (US)

(72) Inventors: Eric Elenko, Boston, MA (US); Michael C. Chen, Norwood, MA (US); LuAnn Sabounjian, Holliston, MA (US)

(21) Appl. No.: 17/932,260

(22) Filed: Sep. 14, 2022

Related U.S. Application Data
(63) Continuation of application No. PCT/IB2021/052299, filed on Mar. 18, 2021.
(60) Provisional application No. 62/991,435, filed on Mar. 18, 2020.
(60) Provisional application No. 63/033,657, filed on Jun. 2, 2020.
(60) Provisional application No. 63/041,614, filed on Jun. 19, 2020.
(60) Provisional application No. 63/087,075, filed on Oct. 2, 2020.
(60) Provisional application No. 63/116,522, filed on Nov. 20, 2020.
(60) Provisional application No. 63/121,175, filed on Dec. 3, 2020.
(60) Provisional application No. 63/152,646, filed on Feb. 23, 2021.

(51) Int. Cl. A61K 31/4418; A61K 9/00; A61P 7/10; A61P 1/16

(52) U.S. Cl. CPC A61K 31/4418; A61K 9/0053; A61P 7/10; A61P 1/16

(57) ABSTRACT

Disclosed herein are methods of treating lymphedema that include administering a clinically effective amount of deupirfenidone.

### TECHNICAL FIELD

The present invention is directed to methods of treating lymphedema with deupirfenidone.

### BACKGROUND

Lymphedema is a chronic debilitating disease of fibrotic and inflammatory origin, that in developed countries such as the United States occurs most often as a complication of cancer treatment. In such cases, lymphedema occurs as a result of iatrogenic injury to the lymphatic system, usually as a result of lymph node dissection or biopsy. Large skin excisions and adjuvant therapy with radiation may also cause lymphedema. See, e.g., Szuba et al., Cancer 95:2260-2267 (2002); Tsai et al., Ann. Surg. Oncol. 16: 1959-72 (2009); Purushotham et al., J. Clin. Oncol. 23: 4312-4321 (2005). According to estimates, as many as 1 in 3 patients who undergo lymph node dissection later develop lymphedema. Conservative estimates suggest that as many as 50,000 new patients are diagnosed annually. See, e.g., DiSipio et al., Lancet Oncol. 14:500-515 (2013); Petrek et al., Cancer 83: 2776-2781 (1998). Because lymphedema is a life-long disease with no cure, the number of affected individuals is increasing annually, with current estimates ranging between 5-6 million Americans (Rockson et al., Ann. NY Acad. Sci. 1131: 147-154 (2008)), and over 200 million people worldwide. It is likely that this number will continue to increase in the future since the development of lymphedema is nearly linearly related with cancer survivorship, and because the prevalence of known risk factors for lymphedema, such as obesity and radiation, is rising. See, e.g., Erickson et al., J. Natl. Cancer Inst. 93: 96-111 (2001).

Lymphedema is disfiguring and debilitating; patients have chronic swelling of the affected extremity, a sense of heaviness, pain, discomfort, skin damage, fibrosis, recurrent infections, limited mobility, and decreased quality of life. See, e.g., Hayes et al., Cancer 118:2237-2249 (2012). Severe symptoms can limit self care. When lymphedema first develops, the skin displays pitting or dimpling, and as the disease progresses, and skin thickening and fibrosis occurs, the skin can have a leathery texture. This non-pitting edema indicates an irreversible stage of lymphedema, in which the skin has a mossy or cobblestoned (hyperkeratotic) appearance. Adipose deposition is a defining characteristic of late-stage lymphedema. Skin in chronic lymphedema is highly susceptible to fissures and recurrent cellulitis. Concurrent cutaneous ulcerations, bacterial and fungal infections, and impetigo, a skin condition resulting in red sores, are also common. Lymphorrhea, an oozing of lymphatic fluid, is also frequently observed. Over time, elephantiasis nostras verrucosa can develop, leading to severe disfiguration of body parts. Cosmetic deformities resulting from lymphedema are difficult to conceal, and psychosocial stigmatization and low self-esteem, depression, anxiety, and negative body image are common among lymphedema patients because of impaired mobility, difficulty fitting into clothing, and deformity of limbs and genitalia.

Additionally, in patients with chronic lymphedema lasting greater than 10 years, there is a 10% risk of developing angiosarcoma, a highly aggressive malignant tumor in the lining of the blood and lymphatic vessels with a poor prognosis and a 5-year survival rate of approximately 35%. Other cancers have been associated with lymphedema as well.

Once lymphedema develops, it is usually progressive. Despite the fact that lymphedema is common and highly morbid, there is currently no cure, and treatment is palliative with a goal of preventing disease progression rather than restoration of lymphatic function. Beaulac et al., Arch. Surg. 137; 1253-1257 (2002). As a result, patients are required to wear tight, uncomfortable garments for the rest of their lives in an effort to prevent lymphatic fluid buildup in the affected extremity, and to undergo intense and time consuming physical therapy treatments. Koul et al., Int. J. Radiat. Oncol. Biol. Phys., 67:841-846 (2007). In addition, despite on-going chronic care, some patients still have severe progression of their disease, with increasing swelling and frequent infections in the lymphedematous limb.

There are currently no approved drug therapies for the treatment of lymphedema. Furthermore, at present, there is no known pharmacologic therapy that can halt progression or promote resolution of lymphedema. Cormier et al., Ann. Surg. Oncol. 19:642-651 (2012). In addition, there has been little progress toward the development of meaningful treatments for lymphatic diseases. Accordingly, development of targeted treatments for lymphedema is an important goal and an unmet biomedical need.

### SUMMARY OF THE DISCLOSURE

It has been discovered that the deuterium enriched pirfenidone LYT-100 (deupirfenidone) has an unexpectedly high tolerability, allowing for higher dosing for greater effectiveness without the adverse effects seen at equivalent doses for pirfenidone. It also allows for dosing without titration to immediately and more effectively treat patients. Further, the unexpectedly high tolerability allows for continuous, long-term, high dose treatment (i.e., without the need to discontinue dosing, interrupt dosing, or titrate the dose down over time due to toxicity of the drug and/or its metabolites). The improved pharmacokinetic profile of LYT-100 relative to pirfenidone further allows for a lower pill burden, and less frequent dosing, e.g., two pills twice a day, with equivalent or significantly enhanced efficacy relative to pirfenidone. LYT-100 has the following structure:

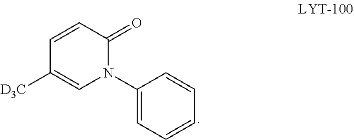 [SMILES: [2H]C([2H])([2H])c1ccc(=O)n(-c2ccccc2)c1]

In one aspect, the invention provides a method of treating lymphedema, comprising administering to a subject in need thereof an effective amount of deupirfenidone:

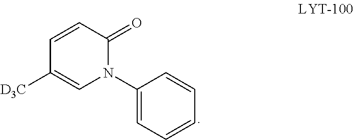 [SMILES: [2H]C([2H])([2H])c1ccc(=O)n(-c2ccccc2)c1]

In some embodiments, the deupirfenidone is administered orally at a total daily dose of 500 mg. In some embodiments, the deupirfenidone is administered orally at a total daily dose of 1000 mg. In some embodiments, the deupirfenidone is administered orally at a total daily dose of 1500 mg. In some embodiments, the deupirfenidone is administered orally at a total daily dose of 2000 mg.

In some embodiments, the deupirfenidone is administered without dose escalation.

In some embodiments, the dosing is twice daily.

In some embodiments, the dosing is three times daily.

In some embodiments, the deupirfenidone is administered without food.

In some embodiments, the deupirfenidone is administered without regard to food.

In some embodiments, the deupirfenidone is administered orally without food in two daily doses of 200 mg each. In some embodiments, the deupirfenidone is administered orally without food in three daily doses of 200 mg each.

In some embodiments, the deupirfenidone is administered orally without food in two daily doses of 250 mg each. In some embodiments, the deupirfenidone is administered orally without food in three daily doses of 250 mg each.

In some embodiments, the deupirfenidone is administered orally without food in two daily doses of 500 mg each. In some embodiments, the deupirfenidone is administered orally without food in three daily doses of 500 mg each.

In some embodiments, the deupirfenidone is administered orally without food in two daily doses of 750 mg each. In some embodiments, the deupirfenidone is administered orally without food in three daily doses of 750 mg each.

In some embodiments, the deupirfenidone is administered orally without food in two daily doses of 1000 mg each.

In some embodiments, the deupirfenidone is administered orally without food in two daily doses of 1000 mg each without dose escalation.

In some embodiments, the deupirfenidone is administered orally without food in two daily doses of 750 mg each without dose escalation.

In some embodiments, the deupirfenidone is administered orally without food in two daily doses of 500 mg each without dose escalation.

In some embodiments, the deupirfenidone is administered orally in two daily doses of 1000 mg each without dose escalation and without regard to food.

In some embodiments, the deupirfenidone is administered orally in two daily doses of 750 mg each without dose escalation and without regard to food.

In some embodiments, the deupirfenidone is administered orally in two daily doses of 500 mg each without dose escalation and without regard to food.

In some embodiments, the deupirfenidone is in tablet form.

In some embodiments, the subject has received treatment for cancer.

In some embodiments, the subject has mild to moderate breast cancer-related lymphedema.

In some embodiments, the subject is receiving or has received chemotherapy or radiation therapy.

In some embodiments, administering the deupirfenidone reduces exposure to a 5-carboxy-pirfenidone metabolite relative to administering pirfenidone. In some embodiments, a Cₘₐₓ of the 5-carboxy-pirfenidone metabolite is reduced by approximately 15%. In some embodiments, an AUC of the 5-carboxy-pirfenidone metabolite is reduced by approximately 25%.

In some embodiments, administering the deupirfenidone results in minimal or no adverse events.

In some embodiments, the pill burden is less than nine capsules or tablets per day. In some embodiments, the pill burden is two, four or six capsules or tablets per day.

In some embodiments, the patient tolerability of the deupirfenidone is improved by greater than 90% as compared to patient tolerability of pirfenidone.

In another aspect is provided a method of treating lymphedema, the method comprising orally administering to a subject in need thereof the deuterium enriched pirfenidone LYT-100, wherein the administering comprises long-term dosing at a high dosage level without interruption.

In some embodiments, the high dosage level is a total daily dose from about 1500 mg to about 2000 mg.

In some embodiments, the long-term dosing is at least 3 months.

In some embodiments, the administering does not comprise up or down titration of the high dosage level during the treating.

In some embodiments, the deupirfenidone is administered with food.

In some embodiments, the deupirfenidone is administered without food.

In some embodiments, the deupirfenidone is administered without regard to food.

In another aspect is provided a method of treating a fibrotic- or collagen-mediated disorder, the method comprising orally administering to a subject in need thereof the deuterium enriched pirfenidone LYT-100, wherein the administering comprises dosing at a high dosage level without interruption.

In some embodiments, the fibrotic- or collagen-mediated disorder is a chronic disease or disorder.

In some embodiments, the chronic disease or disorder is edema, including primary lymphedema and secondary lymphedema.

In some embodiments, the high dosage level is a total daily dose from about 1500 mg to about 2000 mg.

In some embodiments, the long-term dosing is at least 3 months.

In some embodiments, the administering does not comprise up or down titration of the high dosage level during the treating.

In another aspect is provided a method of improving a metabolic profile of LYT-100 over time, the method comprising administering to a subject the deuterium enriched pirfenidone LYT-100 over a period of time of at least 3 months.

In another aspect is provided a method of altering a metabolic profile of LYT-100 over time, the method comprising administering to a subject the deuterium enriched pirfenidone LYT-100 over a period of time of at least 3 months.

In another aspect is provided a method of preventing metabolic adaptation over time by the liver toward metabolism of LYT-100, the method comprising administering to a subject the deuterium enriched pirfenidone LYT-100 over a period of time of at least 3 months.

In another aspect is provided a method of changing a ratio of LYT-100 to at least one metabolite thereof over time, the method comprising administering to a subject the deuterium enriched pirfenidone LYT-100 over a period of time of at least 3 months.

In another aspect is provided a method of altering a metabolic profile of LYT-100 over time, the method comprising administering to a subject the deuterium enriched pirfenidone LYT-100 over a period of time of at least 3 months, and wherein the method reduces one or more of: a Cₘₐₓ of 5-carboxy-pirfenidone; an AUC of 5-carboxy-pirfenidone; a ratio of 5-carboxy-pirfenidone to LYT-100.

In any of these embodiments, the deuterium-enriched pirfenidone LYT-100 is administered with or without food at a total daily dose from about 1000 mg to about 2000 mg. In some embodiments, the deuterium-enriched pirfenidone LYT-100 is administered with or without food at a total daily dose from about 1500 mg to bout 2000 mg. In some embodiments, the deuterium-enriched pirfenidone LYT-100 is administered with or without food at a total daily dose of about 2000 mg.

### BRIEF DESCRIPTION OF THE FIGURES

FIG. 1A illustrates the single-dose pharmacokinetics of an 801 mg dose of LYT-100 and 801 mg dose of pirfenidone over 24 hours. FIG. 1B illustrates an individual's single dose pharmacokinetics of an 801 mg dose of LYT-100 and 801 mg dose of pirfenidone over 48 hours.

FIG. 1C is a model of a 500 mg twice daily dose of LYT-100 (total daily dose of 1000 mg) and its metabolites on day 7. FIG. 1D is a model of a 750 mg twice daily dose of LYT-100 (total daily dose of 1500 mg) and its metabolites on day 7. FIG. 1E is a model of the first 7 days of the dosing of FIG. 1D showing accumulation to steady state. FIG. 1F is a model of a 750 mg once daily dose of LYT-100 (total daily dose of 750 mg) and its metabolites on day 7. FIG. 1G is a model of the first 7 days of the dosing of FIG. 1F showing accumulation to steady state.

FIG. 2 depicts representative micrographs of Sirius-red stained liver sections illustrating that LYT-100 significantly reduced the area of fibrosis.

FIG. 3 illustrates the percent fibrosis area for LYT-100 versus vehicle and control.

FIG. 4A illustrates that LYT-100 does not induce survival of Primary Mouse Lung Fibroblasts (PMFL); FIG. 4B. and FIG. 4C illustrate LYT-100 reduced TGF-β-induced total collagen level in PMFL a 6 well and 96 well format, respectively; and FIG. 4D and FIG. 4E illustrate LYT-100 reduced TGF-β-induced soluble fibronectin levels and soluble collagen levels.

FIG. 5A illustrates that LYT-100 does not affect survival of L929 cells. FIG. 5B, illustrates that LYT-100 inhibits TGF-induced collagen synthesis. FIG. 5C illustrates that LYT-100 significantly inhibits TGF-β-induced total collagen levels. FIG. 5D is a graph illustrating that LYT-100 significantly inhibits TGF-β-induced soluble collagen levels. FIG. 5E illustrates that LYT-100 significantly reduced soluble fibronectin levels, in the absence and presence of TGF-β-induction.

FIG. 6 is a table of Day 1 toxicokinetic parameters from a toxicology study comparing LYT-100 to pirfenidone for parent compound and major metabolites.

FIG. 7 is a table of Day 28 toxicokinetic parameters from a toxicology study comparing LYT-100 to pirfenidone for parent compound and major metabolites.

FIGS. 8A-B are tables of Day 91 toxicokinetic parameters from a toxicology study comparing LYT-100 to pirfenidone for parent compound and major metabolites.

FIG. 9 is a chart of mean plasma concentration of LYT-100 following single and repeat oral administration of deupirfenidone in female Sprague Dawley rats.

FIG. 10 is a chart of mean plasma concentration of pirfenidone (SD-559) following single and repeat oral administration of pirfenidone in female Sprague Dawley rats.

FIG. 11 is a chart of mean plasma concentrations following single and repeat oral administration of 750 mg/kg LYT-100 and pirfenidone in female Sprague Dawley rats on day 28.

FIG. 12 and FIG. 13 are charts depicting the AUC₀₋₂₄ dose relationship following single and repeat oral administration of deupirfenidone in of female and male Sprague Dawley rats, respectively.

FIG. 14 and FIG. 15 are charts depicting the AUC₀₋₂₄ dose relationship following single and repeat oral administration of pirfenidone in of female and male Sprague Dawley rats, respectively.

FIGS. 16A-16D depict results of once daily administration of LYT-100 to reduce swelling in a mouse lymphedema model.

FIG. 17A-17D are charts of mean plasma concentrations in four cohorts (Cohorts 1-4) following oral administration of LYT-100 in human subjects in a multiple ascending dose (MAD) study at 100 mg, 250 mg, 500 mg, and 750 mg BID, respectively.

FIG. 18A and FIG. 18B are charts depicting the concentration of LYT-100 and the three major metabolites in plasma following repeat oral administration of 750 mg BID LYT-100 in human subjects in the MAD study.

FIG. 19 is a table providing Tₘₐₓ, Cₘₐₓ, AUC, and accumulation ratio for the parent drug (LYT-100) and its major metabolites quantitated for Cohorts 1-4 in the MAD study.

FIGS. 20A and 20B (linear and log scale concentration axes, respectively) are charts depicting the concentration of LYT-100 and the three major metabolites (SD-789, SD-790, and SD-1051) in plasma following repeat oral administration of 1000 mg BID LYT-100 in human subjects in the MAD study.

FIG. 21A-21E are charts of mean plasma concentrations for LYT-100 and each metabolite (SD-789, SD-790, and SD-1051) in five cohorts (Cohorts 1-4 and 6) following oral administration of LYT-100 in human subjects in a multiple ascending dose (MAD) study at 100 mg, 250 mg, 500 mg, 750 mg, and 1000 mg BID, respectively.

FIGS. 22A-22F are charts of mean plasma concentrations for LYT-100 and each metabolite (SD-789, SD-790, and SD-1051) in each of the six subjects in Cohort 6 (1000 mg BID).

FIG. 23 is a table of pharmacokinetic data (Tₘₐₓ, Cₘₐₓ, AUC₀₋₁₂, AUC₁₂₋₂₄, AUC₉₆₋₁₀₈, and AUC accumulation ratio (AUC₉₆₋₁₀₈/AUC₀₋₁₂)) for LYT-100 and each metabolite (SD-789, SD-790, and SD-1051) for Cohort 6 (1000 mg BID).

FIG. 24 is a chart depicting the dose proportionality (AUC₉₆₋₁₀₈ versus dose) for LYT-100 and major metabolite SD-789 across Cohorts 1-4 and 6 in the MAD study.

FIG. 25 is a table comparing pharmacokinetic data for LYT-100 and each metabolite (SD-789, SD-790, and SD-1051) with data extrapolated from Huang et al. (200 mg BID Pirfenidone).

FIGS. 26A and 26B are charts of mean plasma concentrations for LYT-100 and each metabolite (SD-789, SD-790, and SD-1051) in plasma following single oral administration of 500 mg LYT-100 in fasted and fed (FIGS. 26A and 26B, respectively) human subjects in Cohort 5 of the MAD study.

FIG. 27 is a table of pharmacokinetic data for LYT-100 and each metabolite (SD-789, SD-790, and SD-1051) for Cohort 5 (500 mg single dose; fed vs fasted).

FIG. 28 is a table of pharmacokinetic data for LYT-100 and the major metabolite (SD-789) extrapolated from the single ascending dose study and compared to the 750 mg cohort in the MAD study.

### DETAILED DESCRIPTION OF THE INVENTION

Pirfenidone (Deskar®), CAS #53179-13-8, Pirespa, AMR-69, Pirfenidona, Pirfenidonum, Esbriet, Pirfenex, 5-methyl-1-phenyl-1H-pyridin-2-one, 5-Methyl-1-phenyl-2-(1H)-pyridone, 5-methyl-1-phenylpyridin-2(1H)-one, is an orally administered small molecule anti-inflammatory and antifibrotic properties. Pirfenidone is an approved therapy for the treatment of IPF in 30 European countries, Japan, South Korea, China, India, Argentina, and Mexico. Worldwide, pirfenidone has been available commercially for IPF since late 2008. Pirfenidone, under the brand name Esbriet®, was approved by the FDA in the United States in 2014 for the treatment of IPF. Pirfenidone has been shown to be generally well tolerated; the most common adverse events (AEs) were gastrointestinal symptoms, fatigue, rash, and photosensitivity reactions.

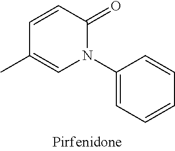

Pirfenidone has been shown to slow the progression of idiopathic pulmonary fibrosis (IPF) in clinical trials (Noble et al. Pirfenidone in patients with idiopathic pulmonary fibrosis (CAPACITY): two randomized trials. Lancet. 2011; 377(9779):1760-1769; Taniguchi et al., Pirfenidone in idiopathic pulmonary fibrosis, Eur Respir J. 2010; 35(4):821-829; King et al. A phase 3 trial of pirfenidone in patients with idiopathic pulmonary fibrosis. N Engl J Med. 2014; 370(22):2083-2092.; Rafii et al., A review of current and novel therapies for idiopathic pulmonary fibrosis. J Thorac Dis. 2013; 5:48-73).

In addition to the treatment of IPF, pirfenidone has been clinically evaluated for its safety and efficacy for the treatment of other chronic fibrotic disorders, including renal fibrosis, hepatic fibrosis, and myelofibrosis. Tada et al., Clin. Exper. Pharmacol. Physiol. 28:522-527 (2001); Cho et al., Clin. J. Am. Soc. Nephrol. 2:906-913 (2007); Nagai et al., Intern. Med. 65 41:1118-1123 (2002); Raghu et al., Am. J. Respir. Crit. Care Med. 159:1061-1069 (1999); Gahl et al., Mal. Genet. Metab. 76:234-242 (2002); Armendariz-Borunda et al., Gut 55:1663-1665 (2006); Angulo et al., Dig. Dis. Sci. 47:157-161 (2002); Mesa et al., Brit. J. Haematol. 114:111-113(2001).

It is likely that multiple mechanisms contribute to the unique profile of pirfenidone. Pirfenidone attenuates fibroblast proliferation, production of fibrosis-associated proteins and cytokines, and biosynthesis and accumulation of extracellular matrix in response to cytokine growth factors (Schaefer et al., Antifibrotic activities of pirfenidone in animal models, Eur Respir Rev. 2011; 20:85-97.; InterMune UK, Ltd. Esbriet® Summary of Product Characteristics. 2011). Pirfenidone blocks the production and activity of TGF-β, a key growth factor that increases collagen production while decreasing its degradation. Moreover, administration of pirfenidone reduces the production of other fibrogenic factors that are induced by TGF-β, such as fibronectin and connective tissue growth factor (Schaefer, 2011). Pirfenidone is capable of blocking bleomycin-induced PDGF production as well as fibroblast and hepatic stellate cell proliferation in response to PDGF (DiSario et al. Effect of pirfenidone on rat hepatic stellate cell proliferation and collagen production, J of Hepatol. 2002 November 37.5.584-591). Pirfenidone inhibits the expression of TNF-α, IL-1, and intercellular adhesion molecule 1 (ICAM-1) (Schaefer, 2011). In a murine macrophage-like cell line, pirfenidone suppressed TNF-α production or secretion through mitogen-activated protein kinase and c-Jun N-terminal kinase-independent mechanisms and increased the levels of IL-10, an anti-inflammatory cytokine (Schaefer, 2011).

In fibrotic diseases such as Idiopathic Pulmonary Fibrosis (IPF), fibrotic pathogenesis is thought to arise from epithelial injury, proliferation of lung fibroblasts, and excessive or inappropriate deposition of connective tissue matrix with endothelin, PDGF, and TGF-β playing critical roles.

Among many other demonstrated effects, pirfenidone attenuates fibroblast proliferation, production of fibrosis-associated proteins and cytokines, and biosynthesis and accumulation of extracellular matrix in response to cytokine growth factors such as TGF-β and platelet-derived growth factor, or PDGF (Schaefer et al., Antifibrotic activities of pirfenidone in animal models, Eur Respir Rev. 2011; 20:85-97.; InterMune UK, Ltd. Esbriet® Summary of Product Characteristics. 2011). Studies have shown that pirfenidone's anti-fibrotic and anti-inflammatory activity is due, at least in part, to inhibition of production and activity of TGF-B. Oku et al., Eur. J. Pharmacol. 590:400-408 (2008); Tian et al., Chin. Med. Sci. J. 21:145-151 (2006); Schaefer et al., Eur. Respir. Rev. 20:85-97 (2011). Pirfenidone is capable of blocking bleomycin-induced PDGF production as well as fibroblast and hepatic stellate cell proliferation in response to PDGF (DiSario et al. Effect of pirfenidone on rat hepatic stellate cell proliferation and collagen production, J of Hepatol. 2002 November 37.5.584-591). Pirfenidone also blocks the production and activity of TGF-β, a key growth factor that increases collagen production while decreasing its degradation. Moreover, administration of pirfenidone reduces the production of other fibrogenic factors that are induced by TGF-β, such as fibronectin and connective tissue growth factor (Schaefer, 2011).

Pirfenidone has demonstrated activity in nonclinical models for multiple fibrotic conditions, including those of the lung, kidney and liver (Schaefer, 2011; Scriabine et al., New developments in the therapy of pulmonary fibrosis, Adv Pharmacol. 2009; 57:419-464). In animal models, pretreatment with pirfenidone reduces inflammation caused by a number of inciting agents, such as lipopolysaccharide (LPS), bleomycin, amiodarone, and carbon tetrachloride, and has demonstrated activity in preclinical models of lymphedema and radiation-induced fibrosis. Pirfenidone has also shown activity investigational clinical studies in patients with unclassifiable interstitial lung disease (uILD), focal segmental glomerulosclerosis (FSGS), and other indications, and has been granted an FDA Breakthrough Therapy designation in ILDs.

Despite pirfenidone's desirable pharmacological profile, it suffers from poor tolerability and pharmacokinetic deficits that limit its use in IPF, and perhaps in other ILDs as well. Pirfenidone is also associated with significant tolerability issues and dose-limiting toxicities.

Pirfenidone has a short half-life in humans and consequently relatively frequent dosing may be required. For the treatment of some conditions, the recommended daily maintenance dose of pirfenidone is 801 mg three times per day (2403 mg·day-1) (a total of nine (9) pills per day at full dose) with a 14-day titration period upon treatment initiation.

In addition, to obtain the maximum benefits of pirfenidone treatment, the adverse events (AEs) associated with pirfenidone require management. The most common AEs are gastrointestinal (GI) and skin-related adverse events, for example, nausea, rash, diarrhea, fatigue, dyspepsia, anorexia, dizziness, gastroesophageal reflux disease, decreased appetite, decreased weight, photosensitivity, and cough. In addition, several treatment-emergent adverse events have been reported, including upper respiratory infection and bronchitis. A recent study in patients treated with pirfenidone under a compassionate use program demonstrated that 44% of the patients had an adverse event with pirfenidone, with only half of them continuing on pirfenidone after a dose-reduction. Raghu & Thickett. Thorax; 68: 605-608 (2013). Adverse events common with pirfenidone at 2403 mg/day include nausea, rash, fatigue, diarrhea, vomiting, dyspepsia, photosensitivity, and anorexia. Noble et al. Lancet; 377: 1760-69 (2011).

The results of several expanded clinical trials are summarized in Lancaster et al., Eur Resp Rev 2017:26:170057 which reports treatment-emergent adverse events (TEAEs) as rates per 100 PEY (equivalent to the frequency at which a physician might expect these TEAEs to occur if 100 patients with IPF were followed for 1 year). Herein, it is noted that the most common reported AEs leading to discontinuation are nausea, fatigue, diarrhea, and/or rash with frequencies as high as 62.1 per 100 PEY (nausea), 27.6 per 100PEY (diarrhea), 52.4 per 100PEY (fatigue). In a single-center, retrospective, observational study of 351 patients who were receiving pirfenidone, 75% of reported AEs were GI-related, with loss of appetite (17%) and nausea/vomiting (15%) being most frequent, similar to what was observed in the phase III trials. The highest number of treatment discontinuations occurred with appetite loss and nausea/vomiting. The incidence of AEs and discontinuation increases with age. The proportion of patients with ADRs leading to dose modification/interruption or discontinuation increased with increasing age: an ADR leading to dose modification/interruption occurred in 32.7% of patients aged ≥80 years and in 18.0% of patients aged <65 years, while an ADR leading to discontinuation occurred in 20.9% of patients aged ≥80 years and in 7.5% of patients aged <65 years.

A long-term observational safety study found that adverse drug reactions led to permanent treatment discontinuation in 28.7% of patients taking pirfenidone. Cottin et al. (2018). Long-term safety of pirfenidone: Results of the prospective, observational PASSPORT study. ERJ Open Research, 4(4), 00084-2018. doi:10.1183/23120541.00084-2018. Real-world experience with pirfenidone in the IPF treatment setting highlights significant problems with treatment compliance, resulting in about half of the patients starting therapy either discontinuing therapy, reducing does, or switching to another therapy, all of which lead to suboptimal disease management. For example, in a large, multinational post-marketing study that analyzed treatment practice of about 11,000 patients diagnosed with IPF, only about 13% of patients were receiving pirfenidone after about a 5-year follow-up period. A high frequency of gastrointestinal adverse events (e.g., nausea, vomiting, and diarrhea) was a major reason for low compliance. Approximately 73% of patients in the study experienced an adverse event, including 38% with gastrointestinal symptoms, leading to the high discontinuation rate.

Several methods for managing AEs associated with pirfenidone are used, including varying the dose titration schedule by using a slower titration schedule and employing dose modifications, including reductions or interruptions (e.g., dose reductions and interruptions may occur in patients receiving pirfenidone, e.g., 28 days' reduction and 14 days' interruption may occur during treatment). In addition, modification of eating habits of the patient may be required when adjusting the pirfenidone dose. Taking pirfenidone with a substantial amount of food, specifically the full dose at the end of a substantial meal or spreading out the three capsules during the meal, may reduce the rate of pirfenidone absorption and mitigate the onset of GI-related AEs.

Although slower titration and dose modification may assist in addressing patient AEs, employing such measures has significant therapeutic impact, notably patients who received pirfenidone 1197 mg/day were reported to experience greater lung function decline than patients who were receiving the full dose of 2403 mg/day.

In addition, pirfenidone treatment has liver function AE's, therefore, monitoring liver function is also important during pirfenidone treatment. Elevations of aspartate transaminase (AST) and alanine transaminase (ALT) levels to >3× the upper limit of normal (ULN) occurred in the phase III trials (3.2%), which were managed by dose modifications or discontinuation. If AST and ALT elevations (>3× to ≤5×ULN) occur without symptoms or hyperbilirubinemia, the dose may be reduced or interrupted until values return to normal. However, in cases in which the AST and ALT elevations (>3× to ≤5×ULN) are accompanied by hyperbilirubinemia or if patients exhibit >5×ULN, pirfenidone must be permanently discontinued.

In addition, patients must be monitored for drug-drug interactions, because the patients taking other oral medications at the same time, may significantly affect pirfenidone metabolism by inhibiting or inducing hepatic enzyme systems (cytochrome P450 1A2 (CYP1A2), CYP3A4, P-glycoprotein). For example, for strong CYP1A2 inhibitors such as fluvoxamine and enoxacin, pirfenidone should be reduced to 267 mg three times daily (801 mg·day-1). For moderate CYP1A2 inhibitors, such as ciprofloxacin at a dosage of 750 mg twice daily, pirfenidone should be reduced to 534 mg three times daily (1602 mg·day-1). Patients should also be assessed for GI intolerance, skin reactions and liver enzyme elevations.

Accordingly, limitations of pirfenidone include: a short half-life of only about 2.5 hours; a high pill burden (of 9 capsules per day (TID dosing); poor tolerability including nausea, diarrhea and photosensitivity; a high dose required for efficacy that induces side effects; and significant interpatient variability. Moreover, pirfenidone treatment requires various AE management strategies, including a slower dose titration for initiating treatment, taking pirfenidone with substantial meals, spacing doses throughout the meal, diet modification, weight-based dosing regimens and dose reductions and interruptions, as well as continual liver function monitoring.

LYT-100, a new chemical entity, is a deuterated, oral small molecule which overcomes the noted challenges associated with pirfenidone (e.g., compliance, dosing and tolerability issues).

Specifically, LYT-100 retains the pharmacology of pirfenidone, but has a differentiated PK profile which enables improved tolerability, less frequent dosing and potentially increased efficacy relative to treatments using pirfenidone.

For example, in a previously conducted single-dose crossover study, described herein in Example 1, an 801 mg dose of LYT-100 resulted in greater drug exposure than an 801 mg dose of pirfenidone (the FDA-approved dose of pirfenidone for treatment of idiopathic pulmonary fibrosis). In this Phase 1 study, LYT-100 was well-tolerated at a dose above 801 mg. These data, together with our PK modeling of LYT-100 and pirfenidone exposures, indicate the potential for twice-a-day dosing with LYT-100.

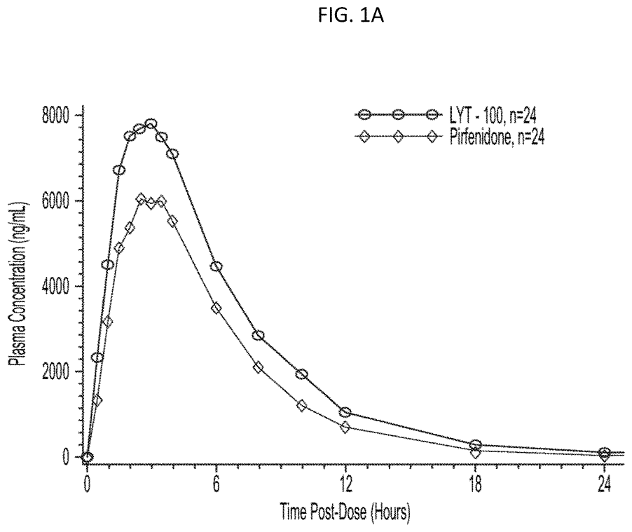

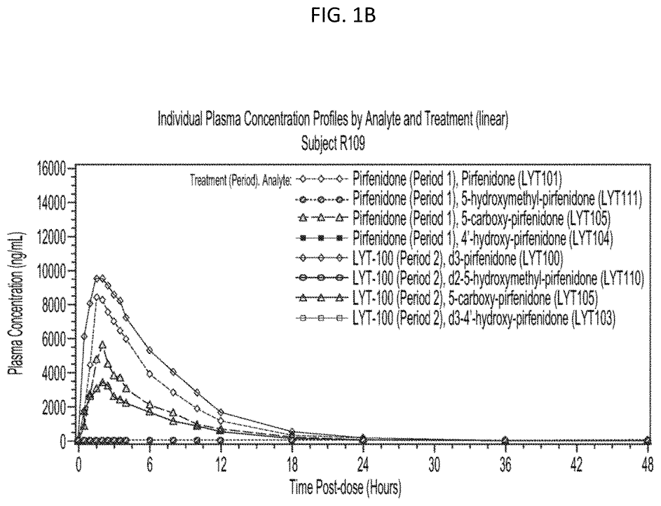

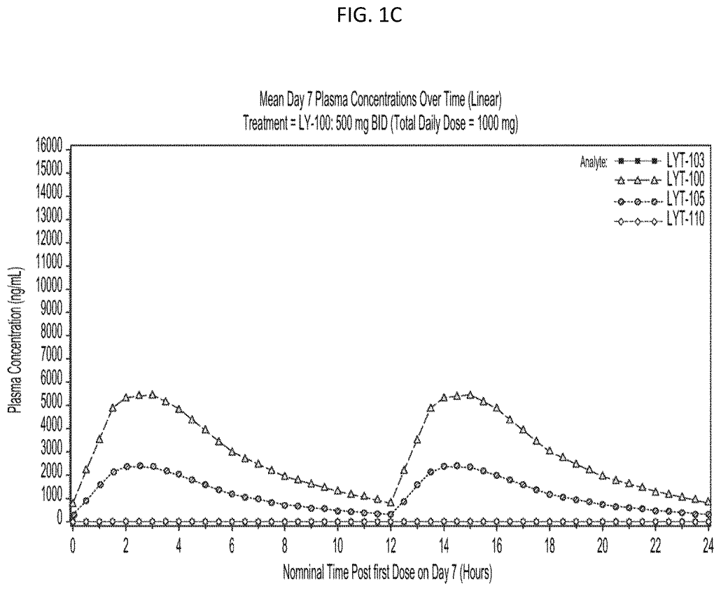

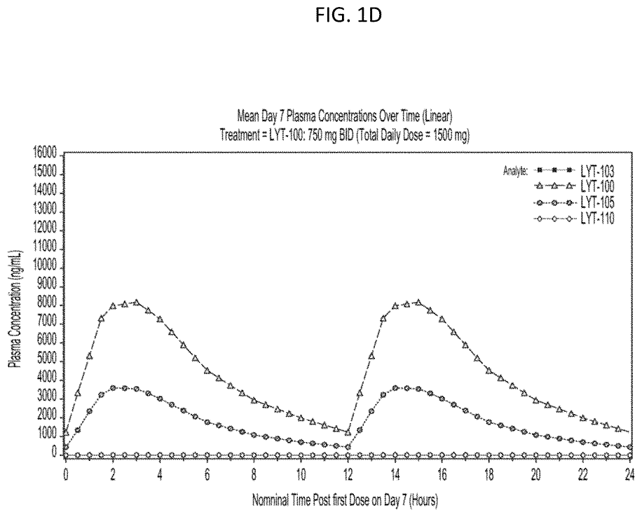

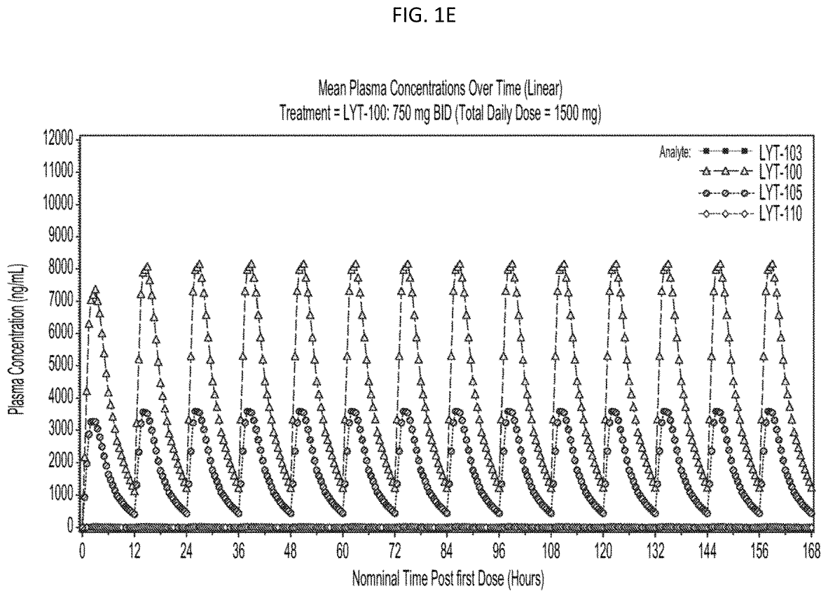

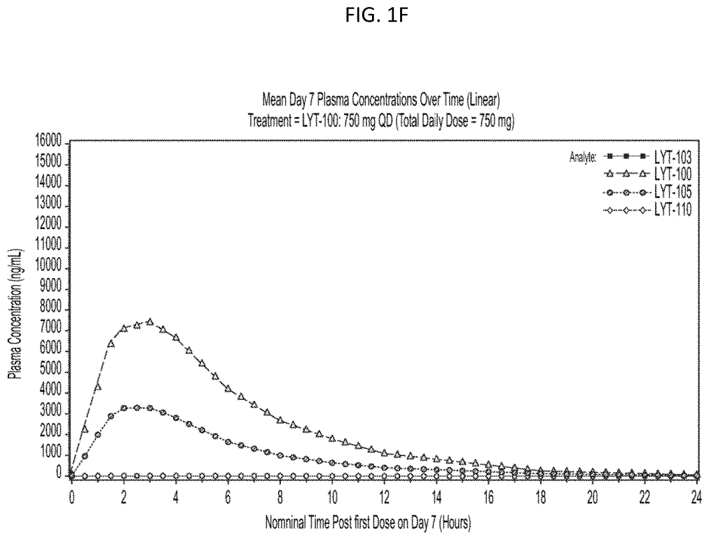

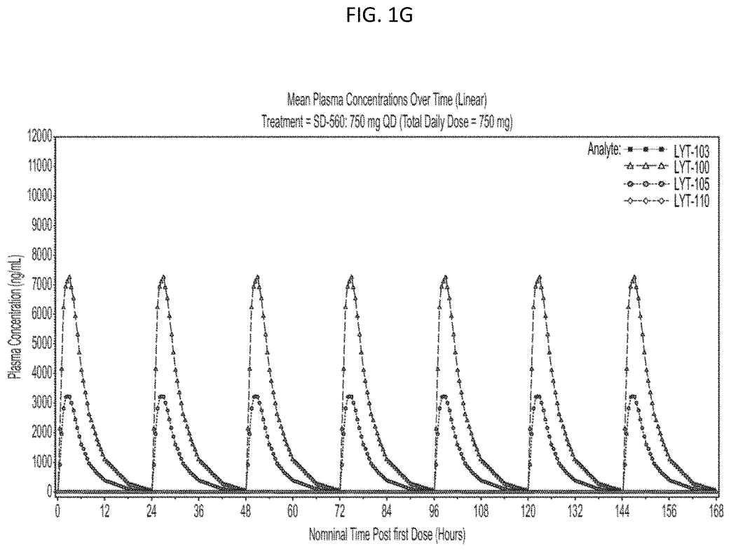

During the MAD study in healthy subjects exemplified herein (Example 2), a discovery was made that provides dosing for increased efficacy and safety in treating lymphedema. Based on the results of comparison with pirfenidone in Example 1, for example, it was believed that the 750 mg dosing of LYT-100 would be the maximum tolerated dosing (750 mg BID; 1500 mg total daily dose) for LYT-100. Specifically, since the Cₘₐₓ of a 750 mg dose of LYT-100 was expected to be at or to exceed that of the Cₘₐₓ of an 801 mg dose of pirfenidone (see e.g., FIG. 1A), the 750 mg dosing was expected to have similar adverse events to those observed with pirfenidone, such that 750 mg of LYT-100 would be the maximum dose that could be tolerated, and in many cases, the LYT-100 dose might even need to be titrated down, much like that for the 801 mg dosing titration utilized for pirfenidone. The 750 mg dosing for LYT-100, however, was surprisingly well tolerated.

The food effect portion of the MAD study evaluated two common PK measures that are used to determine the dose of a product candidate—area under the curve (AUC), which represents exposure, and Cₘₐₓ, which reflects the maximum concentration following drug administration. The LYT-100 AUC and Cₘₐₓ were both observed to decrease with food as compared to fasting conditions. Under fed conditions, the AUC reduction observed with LYT-100 (19%) was comparable to the AUC reduction stated in the ESBRIET® (pirfenidone) US Prescribing Information (16%). The Cₘₐₓ reduction observed with LYT-100 was 23%, while the Cₘₐₓ reduction stated in the ESBRIET® (pirfenidone) US Prescribing Information is 49%.

Overall, the results from the MAD study show that LYT-100 has the potential to offer a tolerability and bioavailability profile that could be highly differentiated at the same exposure levels of pirfenidone, which indicates suitability for use in treating indications where pirfenidone is shown to have benefit but where tolerability concerns limit its use. As noted above, an advantage of LYT-100 is that it can be administered on a twice-a-day dosing schedule, in contrast to pirfenidone, which requires a three-times-a-day dosing schedule. Thus, at least because of a simplified dosing schedule, LYT-100 can engender increased patient compliance and reduced pill burden relative to pirfenidone, and can ultimately be a more effective therapeutic agent. The demonstrated tolerability of LYT-100 at all doses suggests that LYT-100 may be further differentiated from pirfenidone with respect to the potential to avoid dose titration, or at least reduce the duration of any dose titration, and to eliminate the need for interruption or discontinuation of treatment.

Accordingly, disclosed herein is a method of treating a fibrotic-mediated or collagen-mediated disorder, comprising administering to a subject in need thereof the deuterated pirfenidone LYT-100, twice a day. In some embodiments, the fibrotic-mediated or collagen-mediated disorder is a chronic disease or disorder. In some embodiments, the fibrotic-mediated or collagen-mediated disorder is edema, such as primary or secondary lymphedema.

In one aspect, a method of treating lymphedema is provided that includes administering LYT-100 at a dose of 750 mg BID (1500 mg total daily dose) or 1000 mg BID (2000 mg total daily dose). In some embodiments, no titration is required to administer at either of these levels, and the LYT-100 may be administered without regard to food. In view of the enhanced tolerability of LYT-100 relative to pirfenidone, as well as the unique pharmacokinetic profile, it is believed that such high doses may be administered long term (e.g., at least three months, or even indefinitely) without the patient experiencing adverse events, and without the need for any initial dose escalation, downward titration, or interruptions to the dosing.

### Definitions

The term “adverse event” or “AE” refers to any event, side-effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product. Events meeting the definition of an AE include: (1) Exacerbation of a chronic or intermittent pre-existing condition including either an increase in frequency and/or intensity of the condition; (2) New conditions detected or diagnosed after study drug administration that occur during the reporting periods, even though they may have been present prior to the start of the study; (3) Signs, symptoms, or the clinical sequelae of a suspected interaction; (4) Signs, symptoms, or the clinical sequelae of a suspected overdose of either study drug or concomitant medications (overdose per se will not be reported as an AE/SAE).

As used herein, the term “clinically effective amount,” “clinically proven effective amount,” and the like, refer to an effective amount of an API as shown through a clinical trial, e.g., a U.S. Food and Drug Administration (FDA) clinical trial.

“Disorder”, “condition”, “disease”, “syndrome” is meant to be used as interchangeable terms to refer to a biological state that differs from the normal and/or healthy state at the cellular, tissue, organ and/or organism level, e.g., the tissue(s) of one or more organ(s) is affected such that by appearance and/or function it differs from normal and/or healthy tissue. In some embodiments, it refers to an abnormal biological state at the cellular, tissue, organ and/or organism level.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be in numerous dosage forms, for example, tablet, capsule, liquid, solution, soft gel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods, such as mass spectrometry and nuclear magnetic resonance spectroscopy.

The term “is/are deuterium,” when used to describe a given variable position in a molecule or formula, or the symbol “D,” when used to represent a given position in a drawing of a molecular structure, means that the specified position is enriched with deuterium above the naturally occurring distribution of deuterium. In some embodiments, deuterium enrichment is of no less than about 1%, no less than about 5%, no less than about 10%, no less than about 20%, no less than about 50%, no less than about 70%, no less than about 80%, no less than about 90%, no less than about 98%, or in some embodiments no less than about 99% of deuterium at the specified position. In some embodiments, the deuterium enrichment is above 90% at each specified position. In some embodiments, the deuterium enrichment is above 95% at each specified position. In some embodiments, the deuterium enrichment is about 99% at each specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The term “fibrosis” refers to the development of excessive fibrous connective tissue within an organ or tissue.

Terms such as “treating” or “treatment” or “to treat” or “alleviating” or “to alleviate” refer to therapeutic measures that cure, slow down, ameliorate or lessen one or more symptoms of, halt progression of, and/or ameliorate or lessen a diagnosed pathologic condition or disorder. Thus, those in need of treatment include those already with the disorder. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments, a subject is successfully “treated” for a disease or disorder according to the methods provided herein if the patient shows, e.g., total, partial, or transient alleviation or elimination of symptoms associated with the disease or disorder. For example, “treating edema” can include, but is not limited to, decreasing swelling, decreasing inflammation, decreasing fibrosis, decreasing pain, increasing range of motion, decreasing heaviness, decreasing tightness, decreasing skin thickening, and/or improving lymphatic function.

“Prevent” or “prevention” refers to prophylactic or preventative measures that obstruct, delay and/or slow the development of a targeted pathologic condition or disorder or one or more symptoms of a targeted pathologic condition or disorder. Thus, those in need of prevention include those at risk of or susceptible to developing the disorder. Subjects that are at risk of or susceptible to developing lymphedema include, but are not limited to, cancer patients undergoing radiation therapy, chemotherapy, and/or surgical lymph node dissection. In some embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

The terms “subject” and “patient” refers to a mammalian subject, including a human subject. In some embodiments, the patient is human subject.

### Deuterium-Enriched Pirfenidone

In some embodiments, the deuterium-enriched pirfenidone administered is a compound, including LYT-100, or a metabolite thereof, described in WO 2008/157786, WO 2009/035598, WO 2012/122165, or WO 2015/112701, the entireties of which are hereby incorporated by reference.

The compounds may be prepared or isolated in general by synthetic and/or semi-synthetic methods known to those skilled in the art for analogous compounds and by methods described in detail herein. Synthesis of the N-aryl pyridinones of the present invention, including pirfenidone and deuterium-enriched pirfenidone compounds, are described in WO 2008/157786, WO 2009/035598, WO 2012/122165, and WO 2015/112701, the entireties of which are hereby incorporated by reference.

In some embodiments, the present invention includes administering a deuterium-enriched compound shown in Table 1.

TABLE 1
Exemplary Compounds

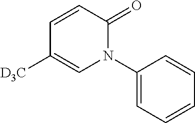 [SMILES: [2H]C([2H])([2H])c1ccc(=O)n(-c2ccccc2)c1]

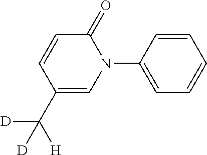 [SMILES: [2H]C([2H])c1ccc(=O)n(-c2ccccc2)c1]

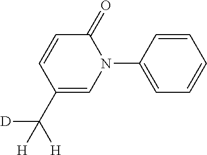 [SMILES: [2H]Cc1ccc(=O)n(-c2ccccc2)c1]

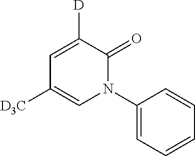 [SMILES: [2H]c1cc(C([2H])([2H])[2H])cn(-c2ccccc2)c1=O]

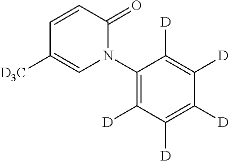 [SMILES: [2H]c1c([2H])c([2H])c(-n2cc(C([2H])([2H])[2H])ccc2=O)c([2H])c1[2H]]

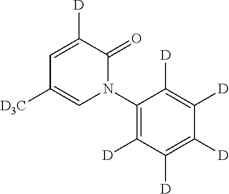 [SMILES: [2H]c1c([2H])c([2H])c(-n2cc(C([2H])([2H])[2H])cc([2H])c2=O)c([2H])c1[2H]]

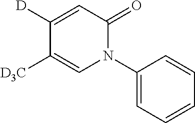 [SMILES: [2H]c1cc(=O)n(-c2ccccc2)cc1C([2H])([2H])[2H]]

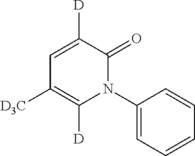 [SMILES: [2H]c1cc(C([2H])([2H])[2H])c([2H])n(-c2ccccc2)c1=O]

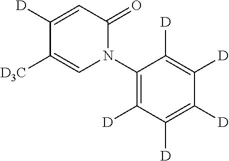 [SMILES: [2H]c1cc(=O)n(-c2c([2H])c([2H])c([2H])c([2H])c2[2H])cc1C([2H])([2H])[2H]]

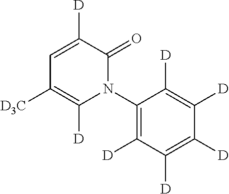 [SMILES: [2H]c1c([2H])c([2H])c(-n2c([2H])c(C([2H])([2H])[2H])cc([2H])c2=O)c([2H])c1[2H]]

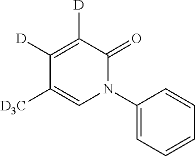 [SMILES: [2H]c1c(C([2H])([2H])[2H])cn(-c2ccccc2)c(=O)c1[2H]]

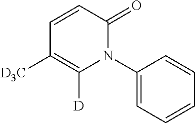 [SMILES: [2H]c1c(C([2H])([2H])[2H])ccc(=O)n1-c1ccccc1]

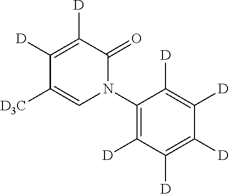 [SMILES: [2H]c1c([2H])c([2H])c(-n2cc(C([2H])([2H])[2H])c([2H])c([2H])c2=O)c([2H])c1[2H]]

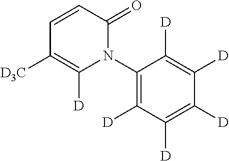 [SMILES: [2H]c1c([2H])c([2H])c(-n2c([2H])c(C([2H])([2H])[2H])ccc2=O)c([2H])c1[2H]]

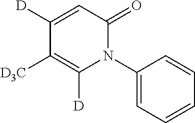 [SMILES: [2H]c1cc(=O)n(-c2ccccc2)c([2H])c1C([2H])([2H])[2H]]

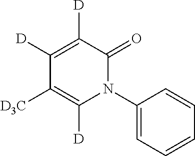 [SMILES: [2H]c1c(C([2H])([2H])[2H])c([2H])n(-c2ccccc2)c(=O)c1[2H]]

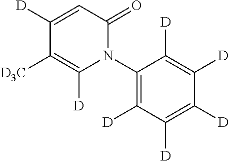 [SMILES: [2H]c1cc(=O)n(-c2c([2H])c([2H])c([2H])c([2H])c2[2H])c([2H])c1C([2H])([2H])[2H]]

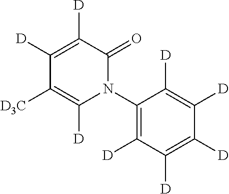 [SMILES: [2H]c1c([2H])c([2H])c(-n2c([2H])c(C([2H])([2H])[2H])c([2H])c([2H])c2=O)c([2H])c1[2H]]

The compounds as disclosed herein can be prepared by methods known to one of skill in the art and routine modifications thereof, and/or procedures found in Esaki et al., Tetrahedron 2006, 62, 10954-10961, Smith et al., Organic Syntheses 2002, 78, 51-56, U.S. Pat. Nos. 3,974,281, 8,680,123, WO2003/014087, WO 2008/157786, WO 2009/035598, WO 2012/122165, or WO 2015/112701; the entirety of each of which is hereby incorporated by reference; and references cited therein and routine modifications thereof.

In some embodiments, the deuterium-enriched pirfenidone is 5-(methyl-d3)-1-phenylpyridin-2-(1H)-one, referred to herein as deupirfenidone or LYT-100, having the following structure:

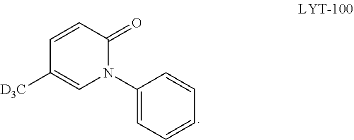 [SMILES: [2H]C([2H])([2H])c1ccc(=O)n(-c2ccccc2)c1]

Reference to “deupirfenidone” or “LYT-100” herein further includes any hydrate, solvate, crystalline polymorph, amorphous form, or the like, of 5-(methyl-d3)-1-phenylpyridin-2-(1H)-one. The preparation of LYT-100 has been disclosed in, for example, U.S. Pat. Nos. 8,383,823 and 9,018,232, and U.S. Patent Application Publication Nos 2009/0131485 and 2013/0018193, each of which is incorporated by reference herein.

As described therein, the level of deuterium enrichment of the pirfenidone may vary. The natural abundance of deuterium is 0.015%, such that each hydrogen atom in non-deuterated pirfenidone naturally comprises about 0.015% deuterium. However, as used herein, reference to “deuterated” means that each atom referred to as “D” is enriched over the natural abundance of deuterium. In some embodiments, the deuterium enrichment at each position designated as D is of no less than about 1%, no less than about 5%, no less than about 10%, no less than about 20%, no less than about 50%, no less than about 70%, no less than about 80%, no less than about 90%. In particular embodiments, each atom referred to as “D” is enriched over the natural abundance of deuterium by at least a factor of 6000 (i.e., a given atom designated as D contains at least about 90% deuterium, with the remainder being hydrogen along with the trace amount of tritium naturally present. For example, each atom designated as D contains at least about 90%, at least about 95%, at least about 96%, at least about 97%, at least about 98%, at least about 99%, at least about 99.5%, or at least about 99.9% deuterium. Thus, while non-isotopically enriched pirfenidone will inherently contain small amounts of deuterated isotopologues, the concentration of naturally abundant stable hydrogen isotopes is small and immaterial as compared to the degree of stable isotopic substitution of compounds of this disclosure. See, for instance, Wada E et al., Seikagaku 1994, 66:15; Ganes L. Zet al., Comp Biochem. Physiol Mol Integr Physiol. 1998, 119:725.

### Metabolites and Pharmacokinetics

Both LYT-100 and pirfenidone share a common major metabolite. An in vitro comparative mass balance study of pirfenidone and LYT-100 in human and rat liver microsomes and a comparative study of pirfenidone and LYT-100 metabolites in rat, dog, mouse, and monkey liver microsomes revealed that metabolism of both compounds was qualitatively similar, and showed that deuteration did not introduce novel metabolites (although metabolism of LYT-100 occurred more slowly).

As demonstrated in the examples herein below, following administration of either LYT-100 or pirfenidone in both rats and humans, the most abundant measured circulating metabolite was 5-carboxy-pirfenidone (LYT-105; SD-789), believed to result primarily through metabolic oxidation by cytochrome P450 subtype 1A2 (CYP1A2). Other metabolites detected include 4′-hydroxy-pirfenidone and the corresponding 4′-hydroxy-deupirfenidone (SD-1051), not quantifiable in humans; as well as 5-hydroxymethyl-pirfenidone (SD-788) and the corresponding 5-(hydroxymethyl-d2)-pirfenidone (SD-790), both minor metabolites in humans.

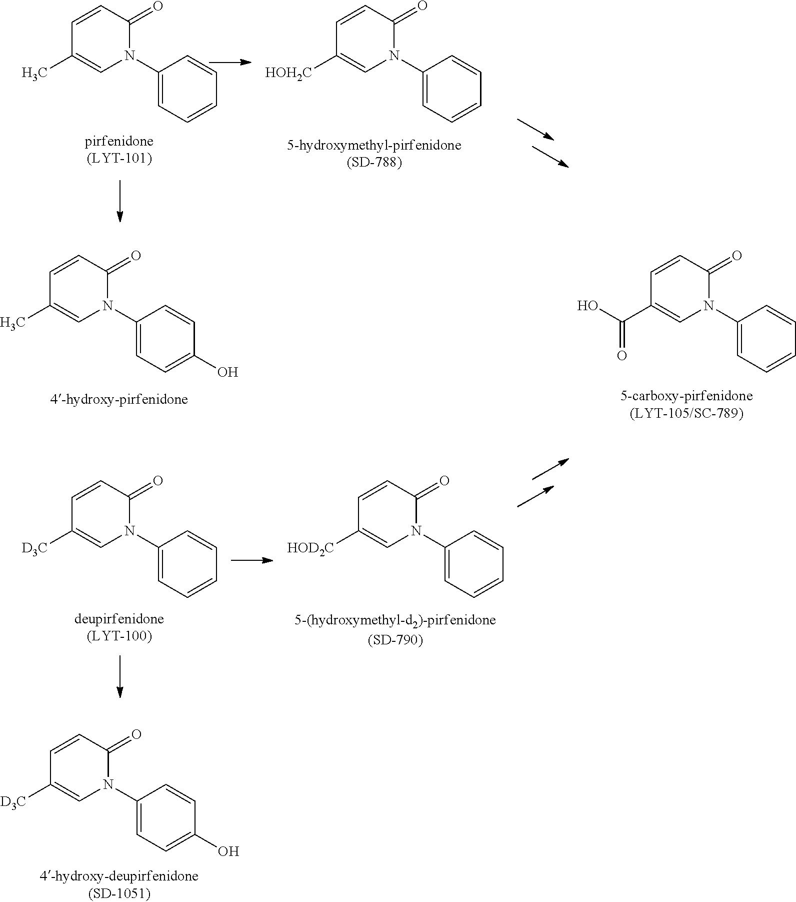

In humans, approximately 80% of an oral pirfenidone dose is excreted in urine, with the majority of recovered drug material (>95%) excreted as the 5-carboxy-pirfenidone metabolite and less than 1% as unchanged pirfenidone (InterMune UK, 2011; Rubino, 2009). In healthy volunteers, the half-life of pirfenidone is approximately 2 to 3 hours (Huang, 2013; Rubino, 2009; InterMune UK, 2011). Plasma exposure to 5-carboxy-pirfenidone is approximately 50% of the exposure to pirfenidone (Huang, 2013). Although the 5-hydroxymethyl-pirfenidone metabolite plasma levels are near or below level of quantification in the majority of human subjects after oral administration of 801 mg pirfenidone (Rubino, 2009), this pharmacologically active metabolite is prevalent in rat (PMDA, 2008). The 5-hydroxymethyl-pirfenidone metabolite has demonstrated anti-inflammatory and anti-fibrotic activity (PMDA, 2008; Togami et al., Possible involvement of pirfenidone metabolites in the antifibrotic action of a therapy for idiopathic pulmonary fibrosis, Biol Pharm Bull. 2013:36:1525-1527) while the 5-carboxy-pirfenidone metabolite has generally been described as pharmacologically inactive (PMDA, 2008; InterMune UK, 2011).

Referring to Example 1, the pharmacokinetics of the active LYT-100 (deupirfenidone) and metabolite 5-carboxy-pirfenidone (LYT-105) were evaluated in human subjects. Administration in the fed state of a single 801 mg dose of LYT-100 resulted in overall greater exposure (AUC, Cₘₐₓ) than observed with administration of an 801 mg dose of pirfenidone. No appreciable difference in the apparent elimination t½ or time to Cₘₐₓ was observed for the 2 compounds. Administration of the 801 mg dose of LYT-100 resulted in greater drug exposure than with the same pirfenidone dose, but surprisingly, the incidence of gastrointestinal and nervous system adverse events was not increased with LYT-100 administration as compared to pirfenidone.

Accordingly, disclosed herein are methods of reducing the level of 5-carboxypirfenidone in a subject, comprising administering a pirfenidone derivative, or a pharmaceutically acceptable salt thereof, to a subject in need thereof. In some embodiments, the level of 5-carboxy-pirfenidone is relative to that in a subject treated with pirfenidone. A pirfenidone “derivative” as used herein generally refers to a pirfenidone molecule that has been functionalized or chemically altered. In some embodiments, the functionalization or chemical alteration includes deuteration, fluorination, or bioisosteric replacement of one or more functional groups. In some embodiments, the functionalization or chemical alteration can include replacement of one or more hydrogen atoms of the 5-methyl group of pirfenidone with deuterium. In some embodiments, the functionalization or chemical alteration can include replacement of one or more hydrogen atoms of the 5-methyl group of pirfenidone with an alkyl group. In some embodiments, the functionalization or chemical alteration can include replacement of one or more hydrogen atoms of the 5-methyl group of pirfenidone with an electron-withdrawing functional group. Electron-withdrawing groups include, but are not limited to, halogen (e.g., fluorine, chlorine, bromine), haloalkyl (e.g., —CF₃, —CF₂H), and —C(O)OR, wherein R is an alkyl or aryl group. Exemplary pirfenidone derivatives include, without limitation, a halogenated derivative of pirfenidone, a deuterium-enriched derivative of pirfenidone, a pirfenidone further substituted on one or more rings, and the like.

Further disclosed herein is a method of reducing adverse events arising from pirfenidone treatment, comprising administering a pirfenidone derivative, or a pharmaceutically acceptable salt thereof, to a subject in need thereof. In some embodiments, the adverse events are reduced by reducing the level of 5-carboxy-pirfenidone in a subject. In some embodiments, the functionalization or chemical alteration includes deuteration, fluorination, or bioisosteric replacement of one or more functional groups. In some embodiments, the functionalization or chemical alteration can include replacement of one or more hydrogen atoms of the 5-methyl group of pirfenidone with deuterium. In some embodiments, the functionalization or chemical alteration can include replacement of one or more hydrogen atoms of the 5-methyl group of pirfenidone with an alkyl group. In some embodiments, the functionalization or chemical alteration can include replacement of one or more hydrogen atoms of the 5-methyl group of pirfenidone with an electron-withdrawing functional group.

Disclosed herein is a pharmaceutical composition comprising a (a) means for reducing the levels of 5-carboxy-pirfenidone in a subject relative to those found in a subject treated with pirfenidone, and (b) a pharmaceutically acceptable carrier.

Also disclosed herein is a pharmaceutical composition comprising a (a) means for reducing the adverse effects associated with pirfenidone treatment, and (b) a pharmaceutically acceptable carrier.

The higher peak and overall exposure of LYT-100 was associated with a lower systemic exposure of the 5-carboxy-pirfenidone (25% Cₘₐₓ reduction; 15% AUC reduction), suggesting the kinetic isotope effect at least partially protects against pre-systemic conversion of pirfenidone into 5-carboxy-pirfenidone. Accordingly, in some embodiments, administration of a deuterated pirfenidone, e.g., LYT-100, reduces the exposure to the 5-carboxy-pirfenidone metabolite. In some embodiments, exposure is reduced relative to pirfenidone by approximately 15% for AUC, 25% for Cₘₐₓ, or both. In some embodiments, the deuterated pirfenidone, e.g., LYT-100, at least partially protects against pre-systemic conversion of pirfenidone into 5-carboxy-pirfenidone.

Referring to Table 2, e.g., on average, after administration of LYT-100, the 5-carboxy-pirfenidone metabolite (LYT-105) represents 43.3% of the parent in comparison to 68.1% of the parent after administration of pirfenidone (Cₘₐₓ). On average, after administration of LYT-100, the 5-carboxypirfenidone metabolite (LYT-105) represented 43.8% of the parent in comparison to 65.9% of the parent after administration of pirfenidone (AUC). This difference in exposure was not associated with a change in half-life, suggesting formation, and not clearance, of this non-deuterated metabolite is affected by the deuterium substitution in the parent molecule.

TABLE 2
Summary of Metabolite Ratio

Metabolite/Parent Ratio %

| | Pirfenidone | |
|---|---|---|
| Pharmaco- | (LYT-101) | LYT-100 |
| kinetic | LYT-105/ | LYT-105/ |
| Parameter | LYT-101 | LYT-100 |
| Cₘₐₓ (ng/mL) | 68.1% | 43.3% |
| AUC_{0-∞} | 65.9% | 43.8% |
| (hr*ng/mL) | | |

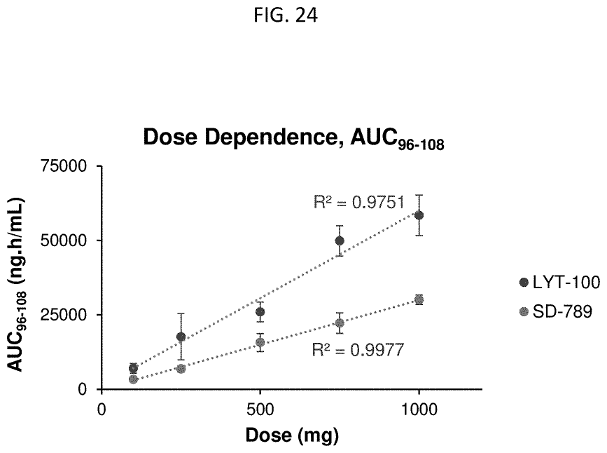

Referring to Example 2, the multiple ascending dose (MAD) study in human subjects, similar results were observed across all dose Cohorts. The major metabolite was 5-carboxy-pirfenidone at all doses, and the average ratio of metabolite to the parent (M/P) by AUC was 0.45. The dose dependence of AUC was evaluated for LYT-100 and 5-carboxy-pirfenidone across all dose Cohorts using the AUC₉₆₋₁₀₈ data points. Linear dose proportionality for both parent and major metabolite was observed. Surprisingly, however, the linear trend of each had different slopes; the parent exposure increased with dose more rapidly than metabolite exposure (FIG. 24).

Data across the Cohorts was compared against data from Huang et al. (“Pharmacokinetics, Safety and Tolerability of Pirfenidone and its Major Metabolite after Single and Multiple Oral Doses in Healthy Chinese Subjects under Fed Conditions.” Drug Res (Stuttg) 63, 388-395; 2013; 200 mg BID pirfenidone), extrapolated to 100, 250, 500, 750, 1000 mg, assuming dose proportionality and comparing to AUC₀₋₁₂ and Cₘₐₓ, LYT-100 and 5-carboxy-pirfenidone metabolite only (Table 3). With the exception of the 500 mg dose, there was an increase for LYT-100 AUC over that of pirfenidone and a decrease for the Cₘₐₓ of the 5-carboxy-pirfenidone metabolite over that of same metabolite from pirfenidone.

TABLE 3
Comparison and Extrapolation of Data

| | | | % difference |
|---|---|---|---|
| | | | (vs. 200 mg |
| | | observed | SD-559) |
| dose (mg) | compound | Cₘₐₓ | Cₘₐₓ |
| 200 | SD-559 | 2305 | N/A |
| (SD-559) | SD-789 | 1476 | N/A |
| 100 | LYT-100 | 1150 | 0% |
| | SD-789 | 567 | −23% |
| 250 | LYT-100 | 2870 | 0% |
| | SD-789 | 1170 | −37% |
| 500 | LYT-100 | 4540 | −21% |
| | SD-789 | 2360 | −36% |
| 750 | LYT-100 | 8190 | −5% |
| | SD-789 | 3370 | −39% |
| 1000 | LYT-100 | 11200 | −3% |
| | SD-789 | 4940 | −33% |

Also surprising is that LYT-100 demonstrated minimal food effect. Referring to Table 4, in the fed state, the AUC of LYT-100 was decreased 19% relative to that achieved when subjects were dosed in the fasted state, and in the fed state, the Cₘₐₓ of LYT-100 was decreased 23% relative to that achieved when subjects were dosed in the fasted state. There was a small increase for Tₘₐₓ.

In the fed state, the AUC of pirfenidone was decreased 15% relative to that achieved when subjects were dosed in the fasted state (similar to LYT-100), the Cₘₐₓ was decreased 49% relative to that achieved when subjects were dosed in the fasted state. There was a large increase of 500% in Tₘₐₓ.

TABLE 4
LYT-100 and Pirefenidone, Fed vs Fasted

| | | | | | % Change Fed |
|---|---|---|---|---|---|
| | Fasted | | Fed | | vs Fasted |
| | LYT-100 @500 mg | | LYT-100 @500 mg | | |
| AUC | 60900 | | 49500 | | −19% |
| Cₘₐₓ | 10300 | | 7900 | | −23% |
| Tmax | 1 | hr | 1.25 | hr | +25% |
| | Pirfenidone @800 mg | | Pirfenidone @800 mg | | |
| AUC | 69.7 | | 59.3 | | −15% |
| Cₘₐₓ | 15.7 | | 7.87 | | −49% |
| Tmax | 0.5 | hr | 3 | hr | +500% |

With respect to the metabolite food effect, surprisingly, there was no food effect on Cₘₐₓ or AUC of the major metabolite 5-carboxy-pirfenidone (<5% change in Cₘₐₓ, AUC; Table 5). There was only a small increase in Tₘₐₓ.

TABLE 5
5-carboxy-pirfenidone for LYT-100
and Pirefenidone, Fed vs Fasted

| | Fasted | Fed | |
|---|---|---|---|
| | LYT-100 @500 mg | LYT-100 @500 mg | % Change Fed |
| | 5-CO2 metabolite | 5-CO2 metabolite | vs Fasted |
| AUC | 2760 | 2610 | −5% |
| Cₘₐₓ | 17100 | 17400 | −1.7% |
| Tmax | 0.75 | 1 | +33% |

In some embodiments, the deuterated pirfenidone, e.g., LYT-100, has at least one of the following properties: a) decreased inter-individual variation in plasma levels of the compound or a metabolite thereof as compared to pirfenidone; b) increased average plasma levels of the compound per dosage unit thereof as compared to pirfenidone; c) decreased average plasma levels of at least one metabolite of the compound per dosage unit thereof as compared to pirfenidone; d) increased average plasma levels of at least one metabolite of the compound per dosage unit thereof as compared to pirfenidone; and e) an improved clinical effect during the treatment in the subject per dosage unit thereof as compared to pirfenidone. Thus, disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound, e.g., LYT-100; so as to effect one or more of a)-e) above during the treatment of the disorder as compared to pirfenidone. In some embodiments, the deuterium-enriched pirfenidone compound has at least two of the properties a) through e) above. In some embodiments, the deuterium-enriched pirfenidone compound has three or more of the properties a) through e) above. In one embodiment, administration of LYT-100 has the properties of increased AUC and Cₘₐₓ compared to pirfenidone; and decreased average plasma levels of 5-carboxy-pirfenidone a compared to pirfenidone. Additionally, in some embodiments, administration of LYT-100 has minimal or no adverse events, or significantly reduced adverse events relative to pirfendione. Additionally, in some embodiments, LYT-100 has an improved clinical effect during the treatment in the subject as compared to pirfenidone.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein; so as to effect decreased inter-individual variation in plasma levels of the compound or a metabolite thereof, during the treatment of the disorder as compared to the corresponding non-isotopically enriched compound. In certain embodiments, the inter-individual variation in plasma levels of the compounds as disclosed herein, or metabolites thereof, is decreased by greater than about 2%, greater than about 5%, greater than about 10%, greater than about 15%, greater than about 20%, greater than about 25%, greater than about 30%, greater than about 40%, or by greater than about 50% (including any numerical increment between the listed percentages) as compared to the corresponding non-isotopically enriched compound.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound, e.g., LYT-100, so as to affect increased average plasma levels of the compound or decreased average plasma levels of at least one metabolite of the compound per dosage unit as compared to the corresponding non-isotopically enriched compound. In certain embodiments, the average plasma levels of the compound as disclosed herein are increased by greater than about 2%, greater than about 5%, greater than about 10%, greater than about 15%, greater than about 20%, greater than about 25%, greater than about 30%, greater than about 40%, or by greater than about 50% (including any numerical increment between the listed percentages) as compared to the corresponding non-isotopically enriched compounds. In certain embodiments, the average plasma levels of a metabolite of the compound as disclosed herein are decreased by greater than about 2%, greater than about 5%, greater than about 10%, greater than about 15%, greater than about 20%, greater than about 25%, greater than about 30%, greater than about 40%, or by greater than about 50% (including any numerical increment between the listed percentages) as compared to the corresponding non-isotopically enriched compounds. Accordingly, in one embodiment, administering LYT-100 results in about a 35% increase in AUC compared to pirfenidone. In one embodiment, administering LYT-100 results in about a 25% increase in Cₘₐₓ compared to pirfenidone. Accordingly, in some embodiments, administration of a deuterated pirfenidone, e.g., LYT-100, reduces the exposure to the 5-carboxy-pirfenidone metabolite. In some embodiments, exposure is reduced relative to pirfenidone by approximately 15% for AUC, 25% for Cₘₐₓ, or both. In some embodiments, the deuterated pirfenidone, e.g., LYT-100, at least partially protects against pre-systemic conversion of pirfenidone into 5-carboxy-pirfenidone.

Plasma levels of the compound as disclosed herein, or metabolites thereof, may be measured using the methods described by Li et al. (Rapid Communications in Mass Spectrometry 2005, 19, 1943-1950).

In some embodiments, the compound has a decreased metabolism by at least one polymorphically-expressed cytochrome P450 isoform in the subject per dosage unit thereof as compared to the non-isotopically enriched compound.

In some embodiments, the cytochrome P450 isoform is selected from CYP2C8, CYP2C9, CYP2C19, and CYP2D6.

In some embodiments, the compound is characterized by decreased inhibition of at least one cytochrome P450 or monoamine oxidase isoform in the subject per dosage unit thereof as compared to the non-isotopically enriched compound.

In certain embodiments, the cytochrome P450 or monoamine oxidase isoform is selected from CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

In some embodiments, the deuterium-enriched pirfenidone, e.g., LYT-100, compound has at least one of the following properties: a) a half-life greater than 2.5 hours; b) a decreased pill burden; c) increased patient tolerability; d) a lower efficacious dose; e) increased bioavailability; f) increased Cₘₐₓ; and g) increase in systemic exposure during the treatment in the subject per dosage unit thereof as compared to the non-isotopically enriched compound. Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to effect one or more of a)-g) above during the treatment of the disorder as compared to the corresponding non-isotopically enriched compound. In one embodiment, administration of LYT-100 results in a half life of greater than 2.5 hours, e.g., between about 2.5 to about 3 hours, or about 3 hours. Additionally, in some embodiments, there is a decreased pill burden including BID dosing as compared to TID with pirfenidone. In addition, LYT-100 has the property of increased patient tolerability, e.g., minimal or no adverse events. In addition, LYT-100 has the property of increased Cₘₐₓ and systemic exposure as compared to pirfenidone.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to effect a longer half-life. In some embodiments, the half-life of the deuterium-enriched pirfenidone compounds as disclosed herein, or metabolites thereof, is increased by greater than about 2%, greater than about 5%, greater than about 10%, greater than about 15%, greater than about 20%, greater than about 25%, greater than about 30%, greater than about 40%, by greater than about 50%, by greater than about 60%, by greater than about 70%, by greater than about 80%, by greater than about 90%, or by greater than about 100% (including any numerical increment between the listed percentages) as compared to the corresponding non-isotopically enriched compound. In some embodiments, the half-life of the deuterium-enriched pirfenidone compounds as disclosed herein, or metabolites thereof, is increased by about 1.5-fold, increased by about 2-fold, greater than about 2-fold, greater than about 3-fold, greater than about 4-fold, greater than about greater than about 5-fold, greater than about 10-fold or more (including any numerical increment between the listed percentages) as compared to the corresponding non-isotopically enriched compound. In one embodiment, the half-life of LYT-100 is increased by greater than about 10%, between 10% and 15%, or about 15% as compared to pirfenidone.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to reduce the pill burden, e.g., effect a pill burden of less than nine (9) capsules per day (TID dosing) of the compound or a metabolite thereof, during the treatment of the disorder as compared to the corresponding non-isotopically enriched compound. Accordingly, in some embodiments, the method includes administering LYT-100 twice as day (BID dosing), as compared the three times a day (TID) for pirfenidone. Additionally, in some embodiments, pill burden is reduced to 2 pills a day, 4 pills a day, or 6 pills a day.

In certain embodiments, the pill burden of the compounds as disclosed herein, is decreased by greater than about 2%, greater than about 5%, greater than about 10%, greater than about 15%, greater than about 20%, greater than about 25%, greater than about 30%, greater than about 40%, or by greater than about 50% (including any numerical increment between the listed percentages) as compared to the corresponding non-isotopically enriched compound. In some embodiments, the pill burden is reduced by greater than about 30% (e.g., 6 pills a day), greater than about 40% (e.g., 4 pills a day), or by greater than about 50% (e.g., 2 pills a day).

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to effect an increased patient tolerability of the compound or a metabolite thereof, during the treatment of the disorder as compared to the corresponding non-isotopically enriched compound. In some embodiments, the patient tolerability is increased by altering the pharmacokinetics, e.g., by increasing the bioavailability (so as to use a lower dose) and/or by extending the half-life of the compound and/or by other means to reduce the side effects of pirfenidone.

In certain embodiments, the patient tolerability of the compounds as disclosed herein, or metabolites thereof, is increased by greater than about 2%, greater than about 5%, greater than about 10%, greater than about 15%, greater than about 20%, greater than about 25%, greater than about 30%, greater than about 40%, by greater than about 50%, by greater than about 60%, by greater than about 70%, by greater than about 80%, by greater than about 90%, or by greater than about 100% (including any numerical increment between the listed percentages) as compared to the corresponding non-isotopically enriched compound. In certain embodiments, the patient tolerability of the compounds as disclosed herein, or metabolites thereof, is increased by about 1.5-fold, increased by about 2-fold, greater than about 2-fold, greater than about 3-fold, greater than about 4-fold, greater than about greater than about 5-fold, greater than about 10-fold or more (including any numerical increment between the listed percentages) as compared to the corresponding non-isotopically enriched compound. In certain embodiments, the patient tolerability of LYT-100 is increased by greater than about 90%, or by about 100% as compared to pirfenidone. In some embodiments, administration of LYT-100 has minimal or no adverse events, or significantly reduced adverse events relative to pirfenidone. In some embodiments, the adverse events are one or more of headache, nausea, abdominal discomfort, abdominal distension, or headache. In certain embodiments, there are no significant adverse events. In certain embodiments, there are no adverse events.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to effect a lower efficacious dose per dosage of the compound or a metabolite thereof, during the treatment of the disorder as compared to the corresponding non-isotopically enriched compound.

In certain embodiments, the efficacious dose per dosage of the compounds as disclosed herein, or metabolites thereof, is decreased by greater than about 2%, greater than about 5%, greater than about 10%, greater than about 15%, greater than about 20%, greater than about 25%, greater than about 30%, greater than about 40%, by greater than about 50%, by greater than about 60%, by greater than about 70%, by greater than about 80%, by greater than about 90%, or by greater than about 100% (including any numerical increment between the listed percentages) as compared to the corresponding non-isotopically enriched compound. In certain embodiments, the efficacious dose per dosage of the compounds as disclosed herein, or metabolites thereof, is decreased by about 1.5-fold, decreased by about 2-fold, greater than about 2-fold, greater than about 3-fold, greater than about 4-fold, greater than about greater than about 5-fold, greater than about 10-fold or more (including any numerical increment between the listed percentages) as compared to the corresponding non-isotopically enriched compound.

Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to increase the bioavailability per dosage of the compound or a metabolite thereof, during the treatment of the disorder as compared to the corresponding non-isotopically enriched compound.

In certain embodiments, the bioavailability per dosage of the compounds as disclosed herein, or metabolites thereof, is increased by greater than about 2%, greater than about 5%, greater than about 10%, greater than about 15%, greater than about 20%, greater than about 25%, greater than about 30%, greater than about 40%, by greater than about 50%, by greater than about 60%, by greater than about 70%, by greater than about 80%, by greater than about 90%, or by greater than about 100% (including any numerical increment between the listed percentages) as compared to the corresponding non-isotopically enriched compound. In certain embodiments, the bioavailability per dosage of the compounds as disclosed herein, or metabolites thereof, is increased by about 1.5-fold, decreased by about 2-fold, greater than about 2-fold, greater than about 3-fold, greater than about 4-fold, greater than about greater than about 5-fold, greater than about 10-fold or more (including any numerical increment between the listed percentages) as compared to the corresponding non-isotopically enriched compound.

In certain embodiments, the systemic exposure per dosage of the compounds as disclosed herein, or metabolites thereof, is increased by greater than about 10%, greater than about 15%, greater than about 20%, greater than about 25%, greater than about 30%, greater than about 35%, greater than about 40%, greater than about 45%, or by greater than about 50% (including any numerical increment between the listed percentages) as compared to the corresponding non-isotopically enriched compound. In one embodiment, the systemic exposure per dosage of the compounds as disclosed herein is increased by greater than about 35% as compared to the corresponding non-isotopically enriched compound. In one embodiment, the systemic exposure per dosage of the compounds as disclosed herein is increased by about 35% as compared to the corresponding non-isotopically enriched compound. In one embodiment, administering LYT-100 results in about a 35% increase in AUC compared to pirfenidone.

Disclosed herein are methods for treating a subject, including a human, having or suspected of having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a deuterium-enriched pirfenidone compound as disclosed herein, or a pharmaceutically acceptable salt, solvate, or prodrug thereof; so as to affect an increase in Cₘₐₓ of the compound per dosage unit as compared to the corresponding non-isotopically enriched compound.

In certain embodiments, the Cₘₐₓ per dosage of the compounds as disclosed herein, or metabolites thereof, is increased by greater than about 10%, greater than about 15%, greater than about 20%, greater than about 25%, greater than about 30%, greater than about 35%, greater than about 40%, greater than about 45%, or by greater than about 50% (including any numerical increment between the listed percentages) as compared to the corresponding non-isotopically enriched compound. In one embodiment, the Cₘₐₓ per dosage of the compounds as disclosed herein is increased by greater than about 25% as compared to the corresponding non-isotopically enriched compound. In one embodiment, the Cₘₐₓ per dosage of the compounds as disclosed herein is increased by about 25% as compared to the corresponding non-isotopically enriched compound. In one embodiment, administering LYT-100 results in about a 25% increase in Cₘₐₓ compared to pirfenidone.

In some embodiments, the method treats the disorder while reducing or eliminating a deleterious change in a diagnostic hepatobiliary function endpoint, as compared to the corresponding non-isotopically enriched compound, e.g., pirfenidone. Disclosed herein are methods for treating a subject, including a human, having or suspected of having a fibrotic-mediated disorder and/or a collagen-mediated disorder (e.g., any of the disorders disclosed herein) or for preventing such disorder in a subject prone to the disorder; comprising administering to the subject a therapeutically effective amount of a compound as disclosed herein, or a pharmaceutically acceptable salt, solvate, or prodrug thereof; so as to reduce or eliminate a deleterious change in a diagnostic hepatobiliary function endpoint, as compared to the corresponding non-isotopically enriched compound. In some embodiments, the diagnostic hepatobiliary function endpoint is selected from alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “gamma-GTP,” “GGT”), leucine aminopeptidase (“LAP”), liver biopsy, liver ultrasonography, liver nuclear scan, 5′-nucleotidase, and blood protein.

### Adverse Event Profile

Without wishing to be bound by any particular theory, it is believed that the improved metabolic stability and/or the attenuated formation of the 5-carboxy-pirfenidone major metabolite seen with LYT-100 may contribute to improved tolerability, less frequent dosing, and better treatment compliance with LYT-100 vs. pirfenidone, all of which may translate to an overall improvement in treatment outcomes. Accordingly, adverse events observed in the MAD study were evaluated and considered with respect to the exposure levels to LYT-100 and the metabolite. Based on the blinded data obtained in the MAD study, all adverse events were mild and transient. Referring to Example 2, the most common events were headache and mild abdominal discomfort. Further, in contrast to pirfenidone (Phase I study, PIPF-005), surprisingly, LYT-100 did not demonstrate a dose-related increase in adverse events based on the MAD data. For example, the low 250 mg BID dose had the most AEs, while the high dose (1000 mg) had the lowest (two incidences of headache). There were no dose limiting toxicities observed, and no maximally tolerated dose was reached.

### Edema and Lymphedema

The term “edema” or “oedema,” as used herein, is an abnormal accumulation of fluid beneath the skin and in body cavities including, but not limited to, limbs, hands/feet, upper body (breast/chest wall, shoulder, back), lower body (buttocks, abdomen), genital (scrotum, penis, vulva), head, neck, or face. The abnormal accumulation of fluid can occur when capillary filtration exceeds lymphatic drainage. In this way, all edema has a lymphatic component. Edema includes lymphedema, lymphatic dysfunction, lymphatic tissue fibrosis, idiopathic edema, peripheral edema, and eye edema. Edema includes acute edema, chronic edema, post-operative edema, gradual-onset edema, primary edema and secondary edema. Chronic edema is edema that has been present for more than three months and can include lymphedema (primary-failure of the lymphatic development and secondary-following damage to the lymphatics), venous edema, chronic swelling due to immobility, edema related to advanced cancer, chronic swelling associated with lymphedema, chronic swelling related to obesity, and chronic swelling associated with rare vascular malformations such as Klippel-Trenaunay syndrome. Symptoms of edema can include accumulation of fluid beneath the skin and in body cavities, swelling, fullness, or puffiness of tissues, inflammation, fibrosis, heaviness, pain, decreased range of motion, aching, recurring infections, skin thickening, or discomfort. In some embodiments, “edema” does not include pulmonary edema or cerebral edema. In some embodiments, the edema is lymphedema. In some embodiments, the lymphedema is primary lymphedema. In some embodiments, the lymphedema is secondary lymphedema.

Lymphedema is a chronic condition that afflicts millions of people and is characterized by severe swelling in parts of the body, typically the arms or legs, due to the build-up of lymph fluid and inflammation, fibrosis and adipose deposition. Lymph is a clear fluid collected from body tissues that transports fats and proteins from the small intestine, removes bacteria, viruses, toxins, and certain proteins from tissues and supplies white blood cells, specifically lymphocytes, to the bloodstream to help fight infections and other diseases. Drainage of lymphatic fluid from tissues is important because the accumulation of lymphatic fluid can be pro-inflammatory. When there is injury to the lymphatics—for example surgery or radiation damage—this can block lymphatic flow and cause pro-inflammatory fluid to accumulate in tissue. This can kick off a vicious feedback loop of inflammation and fibrosis that can cause lymphatic pumping to fail and lymphedema to occur. In lymphedema, the healthy lymphatic fluid homeostasis fails, creating an environment where immune cells begin infiltrating into the tissue and releasing pro-inflammatory and pro-fibrotic cytokines, such as TGF-β. This can create fibrosis of, e.g., arm tissue and the lymphatics themselves. This can further impair lymphatic flow. While the lymphatic system is naturally regenerative, trying to regrow and repair after injury, the feedback loop of fibrosis and inflammation impairs that regeneration.

Lymphedema is a chronic debilitating disease of fibrotic and inflammatory origin, that in developed countries, such as the United States, occurs most often as a complication of cancer treatment. Thus, secondary lymphedema is the most prevalent form of lymphedema, and can develop after surgery, infection or trauma, and is frequently caused by cancer, cancer treatments such as radiation and chemotherapy, trauma or infections resulting in damage to or the removal of lymph nodes. As a complication of cancer treatment, lymphedema occurs as a result of iatrogenic injury to the lymphatic system, usually as a result of lymph node dissection. According to estimates, as many as 1 in 3 patients who undergo lymph node dissection later develop lymphedema. Large skin excisions and adjuvant therapy with radiation may also cause lymphedema. In addition, obesity and radiation are known risk factors for the development of lymphedema.

Lymphedema of the leg and its advanced form, known as elephantiasis, are significant causes of disability and morbidity in areas endemic for lymphatic filariasis, with an estimated 14 million persons affected worldwide (Stocks et al., PLoS Negl Trop Dis. 2015 Oct. 23; 9(10):e0004171). Over 1.1 billion people worldwide are at risk for lymphatic filariasis (Walsh et al, PLoS Negl Trop Dis. 2016 Aug. 22; 10(8):e0004917). Lymphatic filariasis is distributed from Latin America, across central Africa, southern Asia and into the Pacific Islands. Filarial infection is mosquito-transmitted, but efforts to control transmission that are based exclusively on mosquito control have had limited success (Lammie et al., Ann N Y Acad Sci. 2002 December; 979:131-42; discussion 188-96). Wuchereria bancrofti (Wb) is the most widely distributed of the three nematodes known to cause lymphatic filariasis (LF), the other two being Brugia malayi and Brugia timori. Wuchereria bancrofti is the species responsible for 90% of lymphatic filariasis in humans. Filarial infection can cause a variety of clinical manifestations, including lymphoedema of the limbs, genital disease (hydrocele, chylocele, and swelling of the scrotum and penis) and recurrent acute attacks. These acute attacks are caused by secondary infections, to which the lower limbs with lymphatic damage are predisposed, and which are extremely painful and are accompanied by fever. Most infected people do not have symptoms, but virtually all of them have subclinical lymphatic damage and as many as 40% have kidney damage, with proteinuria and hematuria.

Lymphedema is a serious disease with significant health consequences, including disfigurement and debilitation. Patients have chronic swelling of the affected extremity, a sense of heaviness, pain, discomfort, skin damage, fibrosis, recurrent infections, limited mobility, and decreased quality of life.

The protein-rich interstitial fluid accumulation in lymphedema leads to inflammation and an accumulation of fibroblasts, adipocytes, and keratinocytes that transform the initially soft swollen tissue into a hard fibrotic tissue with stiff, thickened skin. Fibrosis is a scarring process, which is characterized by excess deposition of collageneous and non-collagenous extracellular matrix (ECM) due to the accumulation, proliferation, and activation of fibroblasts and myofibroblasts.

Fibroblasts are the main cells that produce, maintain, and reabsorb extracellular matrix (ECM) (reviewed in Kendall and Feghali-Bostwick, Front. Pharmacol., 27 May 2014). Fibroblasts produce the structural proteins of the ECM, expressing different ECMs in different tissues requiring differing degrees of rigidity and flexibility; e.g., fibril rigidity is provided by collagen type I, while expansive stretching ability is provided by elastin proteins. As the major producers of ECM, fibroblasts are also the central mediators of the pathological fibrotic accumulation of ECM and of the cellular proliferation and differentiation that occurs in response to prolonged tissue injury and chronic inflammation in multiple fibrotic diseases including lymphedema.

During initiation and progression of fibrotic disease, such as lymphedema, fibroblasts become activated by inflammatory cytokines and differentiate into myofibroblasts that are characterized by up-regulated cellular migration and a contractile apparatus. Myofibroblasts also display exaggerated ECM production, with increase in the relative production of collagen type I, which stimulates increased chemical signaling secretion and signaling responsiveness. The response is amplified, i.e., cytokines, such as TGFβ1, provide further myofibroblast activation, promoting further collagen deposition, and so forth.

Fibroblasts and myofibroblasts also produce adhesive proteins such as fibronectin and laminin, which form the connection between cells and the ECM and are essential for collagen assembly into ECM. During fibrosis, aberrant fibronectin-matrix assembly is a major contributing factor to the switch from normal tissue repair to dysregulated fibrosis. Although collagen is the most predominant ECM component of fibrotic tissue, excessive deposition of fibronectin also occurs, and precedes the collagen deposition (To and Midwood, Fibrogenesis Tissue Repair. 2011; 4: 21, and references therein). For example, in glomerular and interstitial fibrosis, a significantly elevated expression of total fibronectin is observed, with increased levels of EIIIA+, EIIIB+ and oncofetal (IIICS+) isoforms detected in specific areas of the kidney and in areas of fibrosis.

In addition to ECM deposition, fibroblasts also serve as key players of the immune system with active roles in inflammation and immune cell recruitment (reviewed in Linthout et al., Cardiovascular Research, Volume 102, Issue 2, 1 May 2014, Pages 258-269). On the one hand, fibroblasts drive homing of circulating leucocytes via the release of chemokines, promote the recruitment of circulating leucocytes, and aid retention and survival of immune cells in fibrotic tissue. On the other hand, fibroblasts are activated by components of the innate and adaptive immunity; i.e., they are stimulated chemically by inflammatory agents to differentiate into myofibroblasts with up-regulated rates of matrix production. In other words, fibroblasts can contribute to chronic inflammation, and reciprocally, inflammatory cytokines can promote fibroblast to myofibroblast transition, facilitating fibrosis.

Dysfunctions of the lymphatic system have remained largely untreated or poorly addressed by current therapeutics. There are currently no approved drug therapies for the treatment of lymphedema. Furthermore, at present, there is no known pharmacologic therapy that can halt progression or promote resolution of lymphedema. The current standard of care for lymphedema is management, primarily with compression and physical therapy to control swelling. These approaches are cumbersome, uncomfortable and non-curative, and they do not address the underlying disease, especially in patients with more severe lymphedema. Even with management, some patients will progress from mild-to-moderate lymphedema to more severe forms. In later stages, patients may also seek ablative surgeries, including liposuction or debulking. These surgeries reduce volume but do not restore lymphatic flow, leading to a dependence on compression. An effective treatment for lymphedema cannot just be “mechanical” like the current treatments (e.g., arm compression, pumps, and physical therapy). A treatment for lymphedema would ideally address both inflammation and fibrosis to halt the aforementioned feedback loop and allow the lymphatic system to regenerate.

Given that there are currently no drug therapies that treat the underlying causes of lymphedema, the development of targeted treatments for lymphedema is an unmet biomedical need. LYT-100 is believed to be ideally suited to address lymphedema by virtue of its anti-inflammatory and anti-fibrotic properties, which target the exact mechanisms of the feedback loop that contributes to lymphedema.

There has been little progress toward the development of meaningful treatments for lymphatic diseases. Previous experimental treatments for lymphedema have focused on delivery of lymphangiogenic cytokines. Skobe et al., Nat. Med. 7: 192-198 (2001). For example, some previous studies have focused on repairing damaged lymphatics using lymphangiogenic cytokines such as vascular endothelial growth factor-c (VEGF-C). Tammela et al., Nat. Med. 13: 1458-1466 (2007); Baker et al., Breast Cancer Res. 12:R70 (2010). However, application of this approach, particularly to cancer patients, may be untenable as these same mechanisms regulate tumor growth and metastasis, raising the risk of cancer metastases or recurrence.

In some embodiments, the lymphedema occurs in one or both arms, such as in the hand, wrist, forearm, elbow, upper arm, shoulder, armit, or combination of arm areas or the entire arm. In some embodiments, the lymphedema occurs in one or both legs, such as in the foot, ankle, leg, knee, upper leg or thigh, groin, hip, or combination of leg areas or the entire leg. In some embodiments, the lymphedema occurs in the head, neck, jaw, chest, breast, thorax, abdomen, pelvis, genitals, or other areas of the body cavity. In some embodiments, the lymphedema occurs in one or more limbs, or in one or more limbs and another area of the body.

In some embodiments the lymphedema results from a vascular defect, including venous insufficiency, venous malformation, arterial malformation, capillary malformation, lymphovascular malformation, or cardiovascular disease.

In some embodiments, the subject has or has had cancer, for example, a cancer comprising a solid tumor. In some embodiments, the subject has or has had breast cancer or a cancer affecting female reproductive organs, cutaneous system, musculoskeletal system, soft tissues of the extremities or trunk, male reproductive system, urinary system, or the head and neck. In some embodiments, the subject has undergone axillary lymph node dissection. In some embodiments, the subject has received treatment for cancer, and the edema, lymphedema, or lymphatic injury is associated with the cancer treatment or diagnosis. For example, the subject may be receiving or may have received chemotherapy or radiation therapy for cancer treatment or other indications, or may have had one or more lymph nodes surgically removed in the course of cancer treatment or diagnosis.

In some embodiments, the subject has sustained a lymphatic injury (for example as the result of removal, ligation or obstruction of lymph nodes or lymph vessels, or fibrosis of lymph tissue), or the subject is obese or has or has had an infection that leads to edema, such as lymphedema. In some embodiments, the infection is a skin infection or a history of skin infection related to lymphedema or lymphatic injury. In some embodiments, the infection is a parasitic infection that obstructs lymphatic flow or injures the lymphatic system. In some embodiments, the subject has sustained lymphatic injury from joint replacement, trauma, burns, radiation, or chemotherapy.

Lymphedema typically progresses through multiple stages, with increased fibrosis, limb volume and tissue changes. Of more than 250,000 Americans estimated to be diagnosed with breast cancer each year that undergo surgery, up to one in five will develop secondary lymphedema. Beyond breast cancer, lymphedema can occur in up to 15 percent of cancer survivors with malignancies ranging from melanoma and sarcoma.

A subset of lymphedema patients will also experience cellulitis, a bacterial skin infection that can enter through wounds in lymphedematous skin. Cellulitis often requires hospitalization and intravenous antibiotics to treat, and approximately half of patients with cellulitis will have recurrent episodes. In some embodiments, provided herein are methods for reducing cellulitis in a subject comprising administering an effective amount of LYT-100.

In some rare instances, patients with chronic lymphedema may develop lymphangiosarcoma, a malignant tumor. Lymphedema is classified by clinical staging and severity, as shown in the Table 6 below.

TABLE 6
Clinical Stages of Lymphedema

| | Stage I | Stage II | Stage III |
|---|---|---|---|
| Symptoms | Limb swelling, | Limb swelling, | Disfiguring limb |
| | pitting edema, | skin thickening, | swelling, |
| | limb heaviness | dermal fibrosis, | hyperkeratosis, |
| | and discomfort | fat deposition, | loss of skin |
| | | non-pitting edema | elasticity, |
| | | | skin lesions and |
| | | | overgrowths, |
| | | | massive fibrosis |
| | | | and fat deposition, |
| | | | elephantiasis |

| | |
|---|---|
| Additional | Lifelong need for compression therapy, chronic progression, |
| Clinical | repeated infections (cellulitis, lymphangitis), elephantine |
| Concerns | skin changes, development of lymphangiosarcoma |

In some embodiments, the subject or patient has Stage I lymphedema. In some embodiments, the subject or patient has Stage II lymphedema. In some embodiments, the subject or patient has Stage III lymphedema. In some embodiments, the subject or patient is reduced in stage from Stage III to Stage II or Stage I, or from Stage II to Stage I.

The International Society of Lymphology classifies a lymphedematous limb based on staging that describes the condition of the limb. As the disease progresses into later stages, the affected limb can acquire a “woody texture” due to fibrosis. In addition to clinical staging, clinicians use a measurement of limb swelling to capture disease severity. Cancer treatments lead to new lymphedema patients each year, the majority of which will have mild lymphedema: over 70 percent of patients with secondary lymphedema have milder forms of lymphedema, while the remainder have moderate to severe lymphedema. Table 7 below summarizes the percentage of secondary breast cancer-related lymphedema patients who experience various stages of severity of lymphedema.

TABLE 7
Severity of Secondary Lymphedema

| | Mild | Moderate | Severe |
|---|---|---|---|
| Relative Limb Volume Change | 5-20% | 20-40% | >40% |

| | | |
|---|---|---|
| Percentage Patients | 73% | 27% |

Accordingly, in some embodiments, patients have mild, moderate or severe secondary lymphedema. In some embodiments, patients have mild to moderate secondary lymphedema. In some embodiments, patient have moderate to severe secondary lymphedema. In some embodiments, patients have mild to severe secondary lymphedema. LYT-100 can be used to treat the underlying mechanisms of other forms of secondary or primary lymphedema, for example, lymphatic filariasis.

The natural history of lymphedema is a chronic and progressive disorder, reflected in the increasing severity of limb swelling. The relative increase of limb volume in the affected limb compared to the unaffected limb worsens over time. In patients with mild lymphedema, approximately 48 percent will progress to more severe stages during the first five years of follow-up. Because of the progressive nature of the disease, many patients will progress to the point where bandaging and compression are incapable of reducing limb volume. The potential loss of limb range of motion and function, the risk of secondary infections and complications and the disfigurement result in physical and emotional suffering in cancer survivors. Secondary lymphedema is a lifelong disease and the affected population is increasing each year due to improved survival of cancer patients, changes in patient and disease factors, including obesity, an aging population and increased use of radiation treatment.

In some embodiments, the patients have had breast cancer surgery at least 3, 6, 9, or 12 months prior, and who have completed radiation treatment due to breast cancer at least one, two, three, four, five, six, seven, eight, nine, ten, eleven, or twelve months prior. In some embodiment, they are without recurrent cancer more than 6 months after the breast cancer surgery. In some embodiments, patients are those having pitting edema and at least one of the following: increase in relative limb volume of between 10-20% as measured by the truncated cone method of circumferential tape measurement, or a bioimpedance measure of >+6.5 L-Dex. In some embodiments, patients are also on standard of care compression or have a relative limb volume >10% or L-Dex>14 as compared to pre-surgery and/or pre-radiation volumes.

Bioimpedance, or water content, can be measured via Bioelectrical impedance spectroscopy (BIS). Multiple frequency bioelectrical impedance spectroscopy (BIS) provides accurate relative measures of protein-rich fluid in the upper limb of patients. BIS is a noninvasive technique that involves passing an extremely small electrical current through the body and measuring the impedance (or resistance) to the flow of this current. The electrical current is primarily conducted by the water containing fluids in the body. BIS quantifies the amount of protein-rich fluid in lymphedema by comparison of the affected and non-affected limbs.

Limb Volume (Perometry). Relative limb volume can be measured by the truncated cone method of circumferential tape measurement. Perometry is a noninvasive technique involving a Perometer (Pero-System), which uses infrared light to scan a limb and obtain measurements of the limb's circumference.

Tissue Dielectric Constant (MoistureMeterD). The tissue dielectric constant measures the local tissue water content under the skin at various depths ranging from skin to subcutis. The results are converted into a 0-100% scale to reflect subcutaneous fluid deposition that can occur in early stage lymphedema.

Tissue Firmness (Tonometry/SkinFibroMeter). A tonometer device is pressed into the skin to measure the amount of force required to make an indent in the tissue. The resulting measurement gauges the degree of firmness or fibrosis (tissue scarring) under the skin to assess the severity of lymphedema.

Visual-analogue scales for pain, swelling, discomfort, and function. This graphic scale has a straight line with endpoints from 0 to 10 that is marked by the patient to correlate to their extreme limits of pain, swelling, discomfort and function, ranging from “not at all” to “as bad as it could be.” The higher marks on the line indicates the worse condition.

Lymphedema Symptom Intensity and Distress Survey-Arm (L-SIDS-A) This is a self-report tool consisting of 36 items to evaluate arm lymphoedema and associated symptoms in breast cancer survivors. Symptoms are rated on a ten-point scale (5 points for intensity of the symptom and 5 point for how distressed the patient felt) for heaviness, tightness, pain, stabbing pain, cramping, numbness, achiness, swelling, hardness, tingling, pins and needles, difficulty moving, raising the arm and sadness. Lower scores indicate a higher quality of life.

Lymphedema Quality of Life Tool-Arm (LYMQOL) This is a patient completed questionnaire that assesses upper limb lymphoedema and symptoms and ability to perform common functional activities in patients with BCRL. It covers four domains: symptoms, body image/appearance, function, and mood. It also includes an overall quality of life rating. The overall QOL item ranges from 1-10 Subjects with more severe limb dysfunction have higher scores corresponding to lower quality of life.

In some embodiments, the deuterium-enriched pirfenidone compound (e.g., deupirfenidone; LYT-100) disclosed herein has the ability to effect one or more of the following: a) reduce tissue swelling, b) reduce lymphatic fluid stasis or “pooling,” c) reduce tissue fibrosis, d) reduce tissue inflammation, e) reduce infiltration of leukocytes, f) reduce infiltration of macrophages, g) reduce infiltration of naive and differentiated T-cells, h) reduce TGF-β1 expression and reduce expression and/or activation of downstream mediators (e.g., pSmad3), i) reduce levels of angiotensins and/or ACE, j) reduce collagen deposition and/or scar formation, k) improve or increase lymphatic function, 1) improve or increase lymph fluid transport (e.g., lymphatic flow), m) improve or increase lymphangiogenesis, and/or n) improve or increase lymph pulsation frequency.

### Pharmaceutical Compositions

In another aspect, pharmaceutical compositions are provided for administration in the methods described herein. Pharmaceutical compositions include the active compound, e.g., LYT-100, and one or more pharmaceutically acceptable excipients or carriers. Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Embodiments of the present disclosure can be further defined by reference to the following non-limiting examples. It will be apparent to those skilled in the art that many modifications, both to materials and methods, can be practiced without departing from the scope of the present disclosure.

### EXEMPLIFICATION

Example 1 illustrates the unexpected pharmacokinetic profile of deuterated pirfenidone which provides a significantly reduced pill burden and efficacy at a significantly lower dose, with a significant potential for reducing dose-related side effects and for reduced interpatient variability as compared to pirfenidone. Example 2 provides a dosing and food effect study for deuterated pirfenidone as well as its efficacy in lymphedema. Examples 3, 4, 5, and 6 illustrate the anti-fibrotic and anti-inflammatory efficacy of deuterated pirfenidone.

### Example 1: LYT-100 Increases Systemic Exposure in Humans

LYT-100 was studied in a single dose, double-blinded, cross-over clinical trial of 24 healthy volunteers to assess safety and pharmacokinetics (PK). Following screening, eligible healthy volunteer subjects were admitted to a single clinical study site and were randomized to 1 of 2 treatment sequences. Subjects received a single 801 mg oral dose of either LYT-001 or pirfenidone in Period 1 and, following washout, crossed over to receive the other treatment in period 2. In each period, a standardized breakfast was provided to subjects prior to administration of study drug (to compare the PK profiles in the clinically relevant fed state) and plasma samples were collected over a 48-hour period after dosing for evaluation of PK. Dosing between the 2 periods was separated by a minimum of 7 days. Subjects completed the study upon completion of the 48-hour post-dose assessments following dosing period 2.

To avoid confounding the analysis of results with any influence of formulations, both study drugs (LYT-100 and pirfenidone) were synthesized using the same manufacturing process and were provided as unformulated powder in capsules: LYT-100 801 mg (267 mg capsules×3); and pirfenidone 801 mg (267 mg capsules×3).

All capsules were identical in size, shape, and external color. Both the LYT-100 and the pirfenidone used in this trial were provided in hard-shell gelatin capsules containing 267 mg of either LYT-100 or pirfenidone powder with no excipients. The order of the two treatments was assigned via a randomization schema in a 1:1 ratio such that half of the subjects received LYT-100 first and the other half received pirfenidone first.

The plasma concentrations of LYT-100, pirfenidone, and their respective associated metabolites (e.g., 5-carboxy-pirfenidone, 5-hydroxymethyl-pirfenidone, and 4′-hydroxy-pirfenidone) and sample collection times were used for calculation of the following pharmacokinetic parameters for each subject and treatment:

| | |
|---|---|
| Cₘₐₓ | maximum observed plasma concentration, obtained |
| | directly from the Plasma concentration time profile. |
| tₘₐₓ | time of the maximum observed plasma concentration, |
| | obtained by inspection. If the maximum Plasma |
| | concentration occurs at more than one time point, the |
| | first is chosen. |
| AUC₀₋ₜ | the area under the plasma concentration versus time |
| | curve, from time 0 to the last quantifiable concentration, |
| | calculated by the linear trapezoidal method. |
| λ_{z} | apparent first order terminal elimination rate, obtained |
| | from the slope of the line, fitted by linear least squares |
| | regression, through the terminal points of the log (base |
| | e) concentration-time profiles. |
| t_{1/2} | the apparent first-order terminal elimination half-life, |
| | calculated by the equation t_{1/2} = ln(2)/λ_{z}. |
| AUC_{(inf)} | the area under the Plasma concentration versus time |
| | curve from time 0 extrapolated to infinity, by adding |
| | Cₜ/λ_{z} to AUC₀₋ₜ, where Ct is the last quantifiable |
| | concentration. |
| % AUCₑₓₜᵣₐₚ | the percentage of AUC_{(inf)} obtained by extrapolation, |
| | calculated by Cₜ/λ_{z} expressed as a percentage of the total |
| | AUC_{(inf)}. |
| CL/F | Oral clearance, calculated as (Dose/AUC_{0→∞}) |
| V_{z}/F | volume of distribution during the terminal phase after |
| | oral administration, calculated as (CL/F/λ_{z}) |

FIG. 1A and Tables 8-10 summarize the pharmacokinetics over 24 hours of the active LYT-100 (deupirfenidone) and control pirfenidone (LYT-101), their partially active metabolites, deuterated 5-hydroxymethyl-pirfenidone (LYT-110) and nondeuterated 5-hydroxymethyl-pirfenidone (LYT-111), respectively; and common metabolite, nondeuterated 5-carboxy-pirfenidone (LYT-105). FIG. 1B is an individual's single dose pharmacokinetics of an 801 mg dose of LYT-100 and 801 mg dose of pirfenidone over 48 hours showing LYT-100, pirfenidone and the metabolites of each. The mean Cₘₐₓ values in Table 8 are different when compared with the maximum concentration observed in FIG. 1A. FIG. 1A is a plot of the mean of the concentration values calculated at each (nominal) time point, producing a mean concentration time curve. The mean Cₘₐₓ values are computed from each individual subjects Cₘₐₓ value (which could occur at different Tₘₐₓ times for each individual), i.e., the mean Cₘₐₓ value reported in the PK parameter table is not the Cₘₐₓ for a mean concentration-time curve.

FIG. 1C is a model of a 500 mg twice daily dose of LYT-100 (total daily dose of 1000 mg) on day 7 based on the clinical trial pharmacokinetics. FIG. 1D is a model of a 750 mg twice daily dose of LYT-100 (total daily dose of 1500 mg) on day 7 based on the clinical trial pharmacokinetics. FIG. 1E is a model of the first 7 days of the dosing of FIG. 1D showing accumulation to steady state. FIG. 1F is a model of a 750 mg once daily dose of LYT-100 (total daily dose of 750 mg) on day 7 based on the clinical trial pharmacokinetics. FIG. 1G is a model of the first 7 days of the dosing of FIG. 1F showing accumulation to steady state.

TABLE 8
Summary of Key Pharmacokinetic Parameters for LYT-100 and Pirfenidone.

| Pharmacokinetic | Statistics | Treatment = LYT-100 | | Treatment = Pirfenidone | |
|---|---|---|---|---|---|
| Parameter | (n = 24) | Analyte = LYT-100 | | Analyte = pirfenidone | |
| Cₘₐₓ (ng/mL) | Mean (CV %) | 8835 | (27%) | 7100 | (25%) |
| tₘₐₓ (hr) | Median, (Range) | 2.25 | (1.00-4.00) | 2.50 | (1.50-4.00) |
| AUC₀₋ₜ | Mean (CV %) | 56639 | (46%) | 41091 | (42%) |
| (hr*ng/mL) | | | | | |
| AUC_{(inf)} | Mean (CV %) | 57032 | (46%) | 41316 | (42%) |
| (hr*ng/mL) | | | | | |
| % AUC extrap | Mean (CV %) | 0.674 | (56%) | 0.602 | (54%) |
| Kel (1/hr) | Mean (CV %) | 0.274 | (35%) | 0.300 | (27%) |
| t1/2 (hr) | Mean (CV %) | 2.81 | (31%) | 2.48 | (29%) |
| CL/F (L/hr) | Mean (CV %) | 17.8 | (53%) | 23.5 | (48%) |
| V_{Z}/F( L) | Mean (CV %) | 63.1 | (31%) | 76.5 | (31%) |

TABLE 9
Summary of Key Pharmacokinetic Parameters for Partially Active
Metabolites: deuterated 5-hydroxymethyl-pirfenidone (LYT-110)
and nondeuterated 5-hydroxymethyl-pirfenidone (LYT-111).

| Pharmacokinetic | Statistics | Treatment = LYT-100 | | Treatment = Pirfenidone | |
|---|---|---|---|---|---|
| Parameter | (n = 24) | Analyte = LYT-110 | | Analyte = LYT-111 | |
| Cₘₐₓ | Mean (CV %) | 20.6 | (30%) | 17.1 | (36%) |
| (ng/mL) | | | | | |
| tₘₐₓ (hr) | Median, (Range) | 2.50 | (1.00-4.00) | 2.50 | (1.50-4.00) |
| AUC₀₋ₜ | Mean (CV %) | 111 | (32%) | 75.8 | (42%) |
| (hr*ng/mL) | | | | | |
| AUC_{(inf)} | Mean (CV %) | 124 | (32%) ^{a} | 89.7 | (41%) ^{b} |
| (hr*ng/mL) | | | | | |
| % AUC extrap | Mean (CV %) | 10.1 | (35%) ^{a} | 10.3 | (33%) ^{b} |
| Kel (1/hr) | Mean (CV %) | 0.294 | (44%) ^{a} | 0.367 | (29%) ^{b} |
| t_{1/2} (hr) | Mean (CV %) | 2.76 | (38%) ^{a} | 2.04 | (29%) ^{b} |

Key:
^{a} n = 23,
^{b} n = 19

TABLE 10
Summary of Key Pharmacokinetic Parameters
for 5-carboxy-pirfenidone (LYT-105).

| Pharmacokinetic | Statistics | Treatment = LYT-100 | | Treatment = Pirfenidone | |
|---|---|---|---|---|---|
| Parameter | (n = 24) | Analyte = LYT-105 | | Analyte = LYT-105 | |
| Cₘₐₓ | Mean (CV %) | 3970 | (32%) | 5241 | (31%) |
| (ng/mL) | | | | | |
| tₘₐₓ (hr) | Median, (Range) | 2.50 | (1.50-4.00) | 3.00 | (1.50-4.00) |
| AUC₀₋ₜ | Mean (CV %) | 23090 | (19%) | 26932 | (19%) |
| (hr*ng/mL) | | | | | |
| AUC_{(inf)} | Mean (CV %) | 23350 | (19%) | 27159 | (19%) |
| (hr*ng/mL) | | | | | |
| % AUC extrap | Mean (CV %) | 1.13 | (63%) | 0.843 | (58%) |
| Kel (1/hr) | Mean (CV %) | 0.262 | (33%) | 0.288 | (24%) |
| t_{1/2} (hr) | Mean (CV %) | 2.91 | (30%) | 2.55 | (25%) |

It was observed that the systemic exposure of LYT-100 was about 35% greater than for pirfenidone, and about 25% greater for Cₘₐₓ, with no appreciable difference in the apparent elimination half-life.

The increased systemic exposure to LYT-100 was accompanied by changes in the relative abundance of downstream metabolites. Following LYT-100 and pirfenidone, the most abundant measured circulating metabolite was 5-carboxy-pirfenidone (LYT-105). 5-carboxy-pirfenidone was reduced after LYT-100 relative to pirfenidone by approximately 15% and 25% for AUC and Cₘₐₓ, respectively. As a percent of the parent analyte AUC_{0-∞}, 5-carboxy-pirfenidone represented 43.8% for LYT-100 as compared to 65.9% for pirfenidone (Table 11). The remaining measured metabolites, 5-hydroxymethyl-pirfenidone (LYT-111) and 4′-hydroxy-pirfenidone (LYT-104), were far less abundant, representing less than 2% of parent in terms of AUC. The formation of the metabolite 5-hydroxymethyl-pirfenidone was approximately 50% greater in terms of overall systemic exposure (AUC) after administration of LYT-100. Similarly, 4′-hydroxy-pirfenidone was detectable more frequently after LYT-100 than after pirfenidone. Given the low plasma concentrations of these metabolites, however, these changes contributed little to the overall pharmacokinetic profile of LYT-100 relative to pirfenidone.

TABLE 11
Summary of Metabolite/Parent Ratio, Overall

| | | Metabolite/Parent Ratio^{a} | | | | | |
|---|---|---|---|---|---|---|---|
| | | Pirfenidone (LYT-101) | | | LYT-100 | | |
| Pharmacokinetic | | LYT-105/ | LYT-111/ | LYT-104/ | LYT-105/ | LYT-110/ | LYT-103/ |
| Parameter | Statistic | LYT-101 | LYT-101 | LYT-101 | LYT-100 | LYT-100 | LYT-100 |
| Cₘₐₓ | N | 24 | 24 | 24 | 24 | 24 | 24 |
| (ng/mL) | Mean | 68.1% | 0.2% | 0.0% | 43.3% | 0.2% | 0.1% |
| | Std Dev | 29.0% | 0.1% | 0.0% | 22.2% | 0.1% | 0.0% |
| | CV(%) | 42.6% | 37.5% | 94.5% | 51.2% | 39.1% | 28.0% |
| | Median | 57.5% | 0.2% | 0.0% | 33.5% | 0.2% | 0.1% |
| | Minimum | 36.7% | 0.1% | 0.0% | 22.5% | 0.1% | 0.1% |
| | Maximum | 128.6% | 0.4% | 0.1% | 103.3% | 0.4% | 0.2% |
| AUC_{o-∞} | N | 24 | 19 | 0 | 24 | 23 | 10 |
| (hr*ng/mL) | Mean | 65.9% | 0.2% | — | 43.8% | 0.2% | 0.1% |
| | Std Dev | 28.1% | 0.1% | — | 22.4% | 0.1% | 0.0% |
| | CV(%) | 42.7% | 35.8% | — | 51.2% | 38.0% | 33.8% |
| | Median | 53.7% | 0.2% | — | 32.3% | 0.2% | 0.1% |
| | Minimum | 35.4% | 0.1% | — | 21.2% | 0.1% | 0.1% |
| | Maximum | 128.9% | 0.4% | — | 101.9% | 0.4% | 0.2% |

^{a}The analytes were pirfenidone (LYT-101), nondeuterated 5-hydroxymethyl-pirfenidone (LYT-111), nondeuterated 5-carboxy-pirfenidone (LYT-105), nondeuterated 4′-hydroxy-pirfenidone (LYT-104), LYT-100 (deupirfenidone), deuterated 5-hydroxymethyl-pirfenidone (LYT-110), and deuterated 4′- hydroxy-pirfenidone (LYT-103).
AUC_{o-∞} = area under the plasma concentration versus time curve from zero to infinity; Cₘₐₓ = maximum observed plasma concentration; CV = coefficient of variation; hr = hour; mL = milliliter; MW = molecular weight; N = number; ng = nanogram.
Metabolite/Parent Ratio Formula = Parameter (Metabolite)/Parameter (Parent) * MW(Parent)/MW(Metabolite)
Pharmacokinetic parameters determined using Phoenix WinNonlin v6.3 (Certara)

On average, after administration of LYT-100, the 5-carboxy-pirfenidone metabolite (LYT-105) represented 43.8% of the parent in comparison to 65.9% of the parent after administration of pirfenidone. This difference in exposure was not associated with a change in half-life, suggesting formation, and not clearance of this non-deuterated metabolite is affected by the deuterium substitution in the parent molecule.

Administration in the fed state of a single 801 mg dose of LYT-100 resulted in overall greater exposure (AUC, Cₘₐₓ) than observed with administration of an 801 mg dose of pirfenidone. No appreciable difference in the apparent elimination t½ or time to Cₘₐₓ was observed for the 2 compounds. The higher peak and overall exposure of LYT-100 was associated with a lower systemic exposure of the 5-carboxy-pirfenidone, suggesting the kinetic isotope effect at least partially protects against pre-systemic conversion of pirfenidone into 5-carboxy-pirfenidone.

The deuterium kinetic isotope effect appears independent of phenotype when comparing exposure between deuterated and non-deuterated pirfenidone. CYP1A2 has been reported as the main metabolizing enzyme for pirfenidone and higher enzyme activity in the hyperinduced CYP1A2 phenotype is associated with lower exposure of both deuterated and non-deuterated forms of pirfenidone relative to normal expression levels.

Overall, single doses of LYT-100 and pirfenidone were well tolerated and have a comparable safety profile. No clinically significant differences were observed between the 2 treatments in terms of type, severity, or frequency of treatment emergent adverse events. The most common adverse event following either treatment was headache. Significantly, although administration of the 801 mg dose of LYT-100 resulted in greater drug exposure than with the same pirfenidone dose, the incidence of gastrointestinal and nervous system adverse events was not increased with LYT-100 administration as compared to pirfenidone. No significant changes in laboratory parameters, vital signs, or ECGs were observed following either treatment.

### Example 2: LYT-100 Dosing and Food Effect Study, and Efficacy in Lymphedema

This study is a Phase 1 Multiple Ascending Dose and Food Effect Study in healthy volunteers to determine the pharmacokinetics and maximally tolerated dose of deupirfenidone (LYT-100) followed by a randomized double-blind placebo-controlled phase 2A in patients with breast cancer-related upper limb secondary lymphoedema.

The Multiple Ascending Dose (MAD) and Food Effect (Parts 1 and 2) will be performed at a single Study Centre in Australia. Part 3 will take place at up to 5 study centres in Australia.

### Study Objectives:

This study has 3 Parts, with each Part having specific objectives. Part 1 will assess safety and tolerability in a multiple dose-escalation design, Part 2 is a food effect assessment, and Part 3 is a placebo-controlled assessment of safety and efficacy signals in the target population.

### Parts 1 and 2: Healthy Volunteers

### Primary Objectives

Part 1: To evaluate safety and tolerability of multiple twice-daily (BID) doses of LYT-100 administered over 5 days.

Part 1: To determine time to steady state (up to Day 5) and to characterise the steady-state PK profile of LYT-100.

Part 1: To determine the maximum tolerated dose (MTD) for multiple BID doses of LYT-100 administered over 5 days.

Part 2: To descriptively compare the PK profile and compare the relative bioavailability of a single dose of LYT 100 administered at a dose below the MTD without food, to the equivalent dose with food.

### Secondary Objectives

Part 1: To assess the pharmacokinetic (PK) profiles of multiple BID doses of LYT-100 administered over 5 days for dose proportionality.

Part 3: Breast Carcinoma Patients with Secondary Lymphoedema Following Axillary Node Dissection and/or Sentinel Lymph Node Biopsy

Part 3 is a 26-week randomized, double-blind, placebo-controlled assessment of LYT-100 at multiple study centres. Part 3 will be performed in breast carcinoma patients with secondary mild to moderate lymphoedema following axillary node dissection and/or sentinel lymph node biopsy or excision/clearance, with or without radiation, dosed with LYT-100 (750 mg BID without regard to food) for 26 weeks. Informed consent will be obtained prior to each study part. Screening will be performed up to 21 days prior to administration of the first dose of LYT-100 for all study parts. Only patients who meet all of the applicable inclusion and none of the applicable exclusion criteria per study part will be enrolled.

### Primary Objective

To assess safety and tolerability.

### Secondary Objectives:

To explore efficacy signals of LYT-100 on: lymphatic obstruction and subsequent oedema, infection (as characterised by cellulitis and/or lymphangitis), and quality of life

To assess the population PK profile.

To describe the association between fibrotic and inflammatory biomarkers, in particular TGF-β1, and disease progression in mild to moderate lymphoedema.

### Part 1: Treatment Period

This is a randomised, double-blind, placebo-controlled, multiple ascending dose design to assess the safety, tolerability and PK profile of multiple doses of LYT-100 administered under fed conditions at steady state in healthy subjects. Up to 5 dosing cohorts are planned in Part 1. Planned dose levels are as follows in Table 12.

TABLE 12
Dosing Regimens for Part 1 and Part 2

| | Planned | | | | |
|---|---|---|---|---|---|
| | Dose of | Total Daily | | | |
| | LYT-100 | Dose of | | Number of Participants | |
| Cohort | Part 1 | LYT-100 | | LYT-100 | Placebo |
| 1 | 100 mg | 200 | mg | 6 | 2 |
| | every 12 h | | | | |
| 2 | 250 mg | 500 | mg | 6 | 2 |
| | every 12 h | | | | |
| 3 | 500 mg | 1000 | mg | 6 | 2 |
| | every 12 h | | | | |
| 4 | 750 mg | 1500 | mg | 6 | 2 |
| | every 12 h | | | | |
| (5) | (Part 2 Food | (500 | mg) | (6) from | (2) from |
| | Effect | | | previous | previous |
| | Study) | | | cohort(s) | cohort(s) |
| 6 | 1000 mg | 2000 | mg | 6 | 2 |
| | every 12 h | | | | |

All Part 1 cohorts will be dosed every 12 hours with food for 5 days. Additional cohorts and intermediate doses may be selected in lieu of predefined doses as noted and in accordance with safety and tolerability responses, but doses will not exceed 1000 mg BID or a total daily dose of 2000 mg.

In cohort 6, three sentinel subjects (2 active and 1 placebo) will enrol and dose ≥48 hours in advance of the remaining 5 subjects (4 active and 1 placebo). If clinically significant safety signals assessed as >Mild/Grade 1 are observed in the 3 sentinel subjects in advance of dosing the remaining 5 subjects, the Safety Review Committee may meet to review safety data before the remaining 5 subjects are enrolled.

Up to 40 subjects will be enrolled in Part 1 (n=6 LYT-100 and n=2 placebo in each cohort) unless additional intermediate cohorts are needed. Subjects will be admitted to the Clinical Research Unit (CRU) on Day −1 and will be discharged on Day 7 in the absence of clinically significant safety signals, following completion of all Day 7 assessments and at the Investigator's discretion.

During the treatment period (Day 1 through Day 5), subjects will be administered their assigned study medication BID, every 12 h±0.25 h (with approximately 240 mL of non-carbonated water), 30 minutes after the start of consumption of their standardised breakfast or dinner (12 h apart). A standardised lunch will be served >4 h post breakfast and >4 h prior to dinner. An evening snack will be served >3 h following evening study medication administration. No additional fluids will be allowed during the 1 h pre- and post-doses. On Day 6, subjects will replicate mealtimes as scheduled on Day 1-5. To ensure study drug dosing every 12 hours, here is an example of meal and dosing schedule in Part 1:
Breakfast: meal to be served 30 mins prior to AM dosing. Breakfast must be completed within 30 mins of start time.
Lunch: meal to be served at least 4 h post-AM study drug dose.
Dinner: meal to be served at least 11.5 h post-AM dose and served 30 minutes prior to PM study drug dose.
Evening snack: Snack to be served at least 15 h post-AM dose (at least 3 h post-PM dose).

Subjects will return to the study centre for a follow-up visit 7 days after their final dose of study drug. For all cohorts in Part 1, the decision to escalate or modify the dose prior to dosing of the next Cohort will be determined by a Safety Review Committee (SRC).

### Part 2: Treatment Period

Eight (8) subjects completing Part 1 will participate in Part 2 (n=6 LYT-100 and n=2 placebo). An unblinded statistician will ensure that subjects receiving active treatment or placebo in Part 1 will maintain the same treatment allocation in Part 2, though the dose of active study treatment and number of matching placebo capsules may differ.

A single dose of 500 mg of LYT-100 or placebo will be administered on two days, separated by a minimum 7-day washout period. Four (4) subjects (3 active and 1 placebo) will be randomized to receive their single dose of Part 2 study treatment under fed conditions, while 4 subjects (3 active and 1 placebo) will be randomized to receive their single dose of Part 2 study treatment under fasted conditions.

Subjects will return in Part 2 to the CRU following a minimum 7-day washout period to receive a single dose of 500 mg LYT-100 or placebo after crossing-over to the alternate fasted or fed single dose study treatment administration condition. Subjects will be administered a single dose of their assigned treatment under fasting conditions (Cohort 5) in order to permit a comparison of the rate and extent of absorption of LYT-100 when given the equivalent dose under fed conditions (Cohort 5). The comparison will be based on Day 1, Day 2 and Day 3 PK plasma samples after the first dose of study drug on Day 1 under fast and fed conditions of Part 2.

Rescreening of subjects in Part 2 prior to the first dose of study drug will be conducted according to the Schedule of Events for Part 2 to ensure that the subject continues to meet study eligibility criteria. A total of eight (8) subjects will participate in Part 2 (n=6 LYT-100 and n=2 placebo).

Subjects in Part 2 will receive the same treatment allocation of active or placebo while they were participating in Part 1, though the dose of active study treatment and number of matching placebo capsules may differ. Subjects will be randomized to one of two meal sequences as follows:
Fasted-Fed in first dosing period of Part 1 and second dosing period of Part 2, respectively
Fed-Fasted in first dosing period of Part 1 and second dosing period of Part 2, respectively

Subjects will be admitted to the CRU on Day −1 and will fast overnight for at least 10 h. On Day 1, a single dose of study drug, i.e., 500 mg LYT-100 or placebo will be administered with approximately 240 mL of non-carbonated water while either fasted or fed per randomization sequence.

On Fasted Days, meals will be provided as follows:

On Day 1, breakfast will be provided ≥4 h post-study drug administration. A standardised lunch will be served ≥4 h following breakfast, and dinner will be served ≥4 h following lunch. An evening snack will be served ≥3 h following dinner. No additional fluids will be allowed during the 1 h pre- and post-dose. On Day 2, subjects will replicate mealtimes as scheduled on Day 1.

Mealtimes on Day 1 in relation to dosing in Part 2 are as follows:
Breakfast: meal to be served 4 h post-AM study drug dose.
Lunch: meal to be served at least 4 h following breakfast.
Dinner: meal to be served at least 8 h following breakfast.
Evening snack: snack to be served at least 11 h following breakfast.

On Fed Days, meals will be provided as follows:

On Day 1, a standardised breakfast will be provided 30 minutes prior to study drug administration. A standardised lunch will be served ≥4 h post breakfast and ≥4 h prior to dinner. An evening snack will be served ≥15 h following morning study medication administration. Fluids are restricted only during the 1 h pre- and post-morning dose. On Day 2, subjects will replicate mealtimes as scheduled on Day 1.

Mealtimes on Day 1 in relation to dosing in Part 2 are as follows:
Breakfast: meal to be served 30 mins prior to AM dosing. Breakfast must be completed within 30 mins of start time.
Lunch: meal to be served at least 4 h post-AM study drug dose.
Dinner: meal to be served at least 11.5 h post-AM dose and served 30 minutes prior to PM study drug dose.
Evening snack: Snack to be served at least 15 h post-AM dose

Subjects will remain in the CRU until completion of the 48 h post-dose assessments following the single dose administration with fed or fasted as outlined in the Schedule of Events and in the absence of clinically significant safety signals, following completion of all Day 3 assessments and at the Investigator's discretion. Subjects will return after a ≥7-day washout period to participate in the alternate randomized meal sequence. They will return on Day 10 for their final study day visit.

### Part 1 and Part 2 Safety Monitoring and Follow Up

All subjects in Part 1 and Part 2 will be monitored for safety (including assessment of chemistry, haematology and urinalysis parameters, electrocardiograms [ECGs], vital signs and adverse events [AEs]) and samples will be collected for assessment of PK at predefined time points pre and post-dose as delineated in the Schedule of Events.

All subjects in Part 1 will be followed for at least 30 days and Part 2 will be followed for at least 10 days after the last administered dose of study drug.

In Part 1, subjects will attend the CRU on an outpatient basis 7 days (±1 day) following the last administered dose for safety assessments and a final safety follow up teleconference between site staff and the study participant will occur 30 days (±3 days) after the last administered dose, at the discretion of the Investigator. If required, following the teleconference, an onsite visit to the CRU will be scheduled, at the Investigator's discretion.

In the case of premature discontinuation from the study, subjects will return to the CRU and complete an early termination visit with assessments as delineated in the Schedule of Events. Following the early termination visit, subjects will be contacted by telephone by site staff 30 days (±3 days) in Part 1 and 10 days (±3 days) in Part 2, post the last administered dose of study drug for a final safety review, at the Investigator's discretion.

### Part 3 Treatment Period

Part 3 is a double-blind, parallel, placebo-controlled study being conducted to evaluate the safety and efficacy of LYT-100 compared to placebo. Part 3 will be conducted across multiple centres, with up to 50 patients randomized to receive LYT-100 or placebo, in a ratio of 1:1.

Dosing is determined from outcomes in Part 1 and Part 2. LYT-100 dosing is to be titrated starting at 500 mg BID during the first 3 days of dosing, followed by 750 mg BID thereafter, or matching placebo. Patients will take double blind-study medication orally without regard to food BID (approximately 10 to 12 hours between the two daily doses) on an outpatient basis for 26 weeks. Patients will be followed for an additional 22 weeks post-treatment to assess for longer-term outcomes.

TABLE 13
Dosing Regimens for Days 1 through 3 and Thereafter

| Day 1 to Day 3 | Day 4 through Week 26 |
|---|---|
| ¹LYT-100 500 mg BID² or | ¹LYT-100 750 mg BID² or |
| matching Placebo x 3 days | matching Placebo x 179 days |

¹Patients will be administered LYT-100 study medication, or placebo, orally without regard to food with approximately 10 to 12 hours between the two daily doses.
²Doses may be adjusted according to safety and tolerability to avoid toxicity by adjusting to lower doses in response to patient safety and tolerability issues. If dosing titration is not well tolerated, adjustments to dosing may be made as follows: reductions to 250 mg, BID X 2 days (may be longer if needed), 500 mg BID X 2 days (may be longer if needed), 750 mg BID thereafter vs. matching placebo. In addition, if tolerability issues persist, the patient may be instructed to take study medication with food.

Patients with breast cancer related lymphoedema will be assessed for safety, tolerability, clinical endpoints, PK, and biomarkers while receiving LYT-100 or placebo over a 6-month dosing period. If a patient is using a standard of care compression sleeve, compression pump therapy, and/or manual lymphatic drainage within 4 weeks prior to screening, they must be agreeable to continuing the same routine care throughout the 6-month study treatment period and throughout 2 weeks post-study drug discontinuation. Qualified patients currently using a compression sleeve at least 4 weeks prior to screening should be properly fitted for a new compression sleeve and begin wearing this at least 1 week prior to their baseline visit/assessments. If a patient is not using a standard compression sleeve, compression pump therapy, and/or manual lymphatic drainage ≥4 weeks prior to screening and are not planning to be using these prior to the study, they must be agreeable to not using the lymphoedema therapy(s) throughout treatment and 2-weeks post-study drug discontinuation. Patients will be stratified at enrolment into the standard compression sleeve, compression pump therapy, and/or manual lymphatic drainage stratum vs. non-compression/non-lymphatic drainage stratum. In addition, patients will be stratified by higher risk of lymphoedema progression (axillary lymph node dissection) vs. lower-risk of lymphoedema progression (sentinel node biopsy).

TABLE 14
Part 3 Baseline Randomization Stratification Levels
Compression sleeve, compression pump, and/or lymphatic drainage

| Yes | | No | |
|---|---|---|---|
| Risk for Progression | | Risk for Progression | |
| High | Low | High | Low |
| (history of axillary | (history of | (history of axillary | (history of |
| lymph node | sentinel | lymph node | sentinel |
| dissection and/or | node | dissection and/or | node |
| regional lymph node | biopsy) | regional lymph node | biopsy) |
| radiation posterior | | radiation posterior | |
| field with or without | | field with or without | |
| supraclavicular) | | supraclavicular) | |

Following confirmation of study eligibility, patients will be seen in the clinic for their final baseline assessments (Day −1). Patients will begin their BID dosing of study medication on the following morning (Study Day 1) and will continue for 26 weeks, with clinic visits at Weeks 1, 2, 4, 8, 12, 16, 20 and 26. The site will contact the patient by phone at Week 23 to check in and assess for compliance to study drug, assess for new concomitant medications and adverse events and remind the patient to complete their Patient Diary one week prior to the Week 26 study visit.

All patients in Part 3 will be monitored for safety (including assessment of chemistry, haematology and urinalysis parameters, ECGs, vital signs and AEs). Patients will complete the Efficacy assessments which will include clinical and quality of life measures at time points as delineated in the Schedule of Events. Sparse PK samples will be obtained for population PK analysis to determine the variability of LYT 100 drug concentration data in individual patients across multiple clinical sites. Fibrotic and inflammatory biomarkers will be assessed for changes from baseline. With patients using compression sleeves, pumps and/or lymphatic drainage as a treatment modality(s) at least 4 weeks prior to and at Screening, they will remain on compression treatments during the treatment period as noted. Compression sleeves will be removed upon arrival at each study visit and until after the bioimpedance assessment is collected which should be scheduled toward the end of the study visit and just prior to blood pressure and blood collection. Time of sleeve removal will be noted at each study visit. In addition to routine practices such as diet, exercise, or skin care, use or non-use of compression sleeves, compression pumps and/or lymphatic drainage will be recorded in the Patient Diary one week prior to each study visit with lymphoedema assessments, including frequency of use and the number of hours used on each occasion. Medication compliance will also be recorded on the Patient Diary.

### Part 3 Follow Up

Safety and tolerability will be assessed throughout all parts of the study by monitoring AEs, physical examination, vital signs, 12-lead ECGs, clinical laboratory values (haematology panel, multiphasic chemistry panel and urinalysis), and review of concomitant treatments/medication use.

Patients will return to the clinic 28±3 days from the last dose of study medication for a safety follow-up visit as delineated in the Schedule of Events.

In the event of premature discontinuation from the study, patients will return to the investigative site and complete an early termination visit with assessments as delineated in the Schedule of Events. Following the early termination visit, patients will be asked to return to the investigative site for one last safety follow-up visit, approximately 28-days from the last dose of study medication.

### Safety Oversight for Part 1 and Part 2:

The study will be subject to oversight by a SRC comprised of the Principal Investigator (PI), medical monitor (MM), and Sponsor representative, at a minimum. Details of the roles and functioning of the SRC will be available in the SRC Charter.

### Part 1

The SRC will provide recommendations on the dose of LYT-100 for the next Cohort. The raw data will remain blinded. In the event that unblinding of an individual subject is required, every effort will be made to not compromise the overall blinded status of the study.

If an intolerable dose of LYT-100 is identified, the dose will not be further escalated and the previously tolerated dose will be considered the MTD or alternatively, an intermediate dose, lower than the intolerable dose, may be explored.

Escalation from one dose level to the next will occur after review of raw clinical safety data and approval by the SRC up to and including the onsite follow-up visit on Day 12 (7 days post last administration of study drug) for the last subject in the preceding cohort; cumulative data for earlier cohorts may also be reviewed.

As noted previously, in Cohort 6, three sentinel subjects (2 active and 1 placebo) will enrol and dose ≥48 hours in advance of the remaining 5 subjects (4 active and 1 placebo). If clinically significant safety signals assessed as >Mild/Grade 1 are observed in the 3 sentinel subjects in advance of dosing the remaining 5 subjects, the Safety Review Committee may meet to review safety data before the remaining 5 subjects are enrolled.

### Part 2

A single dose of 500 mg of LYT-100 or placebo will be administered on two days, separated by a minimum 7-day washout period.

Stopping Rules for Part 1 and Part 2:

At any phase of the study, administration of study drug will be paused and subjects will not receive further study drug until data review, recommendations and approval have been provided by the SRC.

Dose-limiting toxicity will be defined as 2 or more clinically significant AEs or abnormal laboratory values assessed as unrelated to intercurrent illness, or concomitant medications, which are determined by the Investigator to be related to the study drug and meet any of the following criteria:
Two or more Grade 3 toxicities that are considered possibly or probably related to study drug.
Any subject experiences a serious adverse event (SAE) that is considered possibly or probably related to study drug, or
An AE or group of AEs that singularly or in aggregate suggests to the Investigator, Sponsor or Medical Monitor that the study drug is possibly or probably related, and it is poorly tolerated and further treatment per protocol may not be safe.

With observation of apparent dose-limiting toxicity, review by the SRC will take place as soon as possible to evaluate the events and determine next steps.

### Safety Oversight for Part 3

This is a phase 2A, multi-centre, double-blind, parallel arm, placebo-controlled study of LYT-100 in patients with breast cancer-related lymphoedema. The study will be performed in up to 5 clinical sites in Australia.

The SRC will convene after 20% of the patients enrolled in Part 3 have completed Week 8 of the Treatment Phase and the double-blind data is available for review. If safety or tolerability issues are identified by the medical monitor for patients while receiving LYT-100 vs. placebo at any time in Part 3, the SRC may meet again to review safety and available population PK data and provide recommendations. Options for changes to dosing or protocol assessments may be recommended by the SRC. Ultimate decisions regarding those recommendations remain with the Sponsor. Adverse events of special interest including elevated liver enzymes (e.g., ALT, AST, total bilirubin elevations), photosensitivity and rash, and gastrointestinal symptoms (e.g., nausea, vomiting diarrhea, dyspepsia, gastroesophageal reflux and abdominal pain) will be reviewed by the medical monitor periodically for changes in IP tolerability throughout the trial and if warranted, may trigger additional ad hoc SRC meeting(s).

### Number of Participants (Planned):

Parts 1 and 2: Up to 48 healthy female and male adult subjects (3:1 ratio), unless additional intermediate cohorts are needed.

Part 3: Up to 50 patients with breast cancer-related upper-limb unilateral secondary lymphoedema (1:1 ratio).

### Main Criteria for Inclusion

### Part 1 (MAD) and Part 2 (Single-Dose Fed-Fasting)

### Healthy Volunteers

Inclusion Criteria:
1. Male or female between 18 and 65 years old (inclusive) at the time of screening.
2. In good general health at screening, free from clinically significant unstable medical, surgical or psychiatric illness, at the discretion of the Investigator.
3. Subjects have a body mass index (BMI) between ≥18.0 and ≤35.0 kg/m2 at screening.
4. Vital signs (measured in supine position after 5 minutes' rest) at screening:
5. Systolic blood pressure ≥90 and ≤140 mmHg;
6. Diastolic blood pressure ≥40 and ≤90 mmHg;
7. Heart rate ≥40 and ≤100 bpm;
8. Temperature ≥35.5° C. and ≤37.5° C.;
9. Vital signs may be repeated once, within a minimum of 10 minutes of the completion of the last set of vital signs (while maintaining supine position until the repeated set of vital signs are collected), if it is suspected that falsely high or low levels have been obtained.
10. No relevant dietary restrictions, and willing to consume standard meals provided and willing to avoid soy products while participating in the trial.
11. Willing to comply with all study procedures and requirements, including not driving or operating machinery for 12 h following study drug administration.
12. Willing to abstain from direct sun exposure from 2 days prior to dosing and until final study procedures have been conducted.

### Main Criteria for Exclusion

### Part 1 (MAD) and Part 2 (Single-Dose Fed-Fasting)

### Healthy Volunteers

1. History or presence of malignancy at screening or baseline, with the exception of adequately treated localised skin cancer (basal cell or squamous cell carcinoma) or carcinoma in-situ of the cervix.
2. Clinically significant infection within 28 days of the start of dosing, including a positive test for COVID-19, or infections requiring parenteral antibiotics within the 6 months prior to screening. Known exposure to another person with COVID-19 within the last 14 days is also an exclusion criterion.
3. Clinically significant surgical procedure within 3 months of screening, at the discretion of the Investigator.
4. Currently suffering from clinically significant systemic allergic disease at screening or baseline or has a history of significant drug allergies including a history of anaphylactic reaction (particularly reactions to general anaesthetic agents); allergic reaction due to any drug which led to significant morbidity; prior allergic reaction to pirfenidone.
5. Chronic administration (defined as more than 14 consecutive days) of immunosuppressants or other immune-modifying drugs within 3 months prior to study drug administration; corticosteroids are permitted at the discretion of the Investigator.
6. History or presence at screening or baseline of a condition associated with significant immunosuppression.
7. Positive test for hepatitis C antibody (HCV), hepatitis B surface antigen (HBsAg), or human immunodeficiency virus (HIV) antibody at screening.
8. Symptoms of dysphagia at screening or baseline or known difficulty in swallowing capsules.
9. Any condition at screening or baseline (e.g., chronic diarrhoea, inflammatory bowel disease or prior surgery of the gastrointestinal tract) that would interfere with drug absorption or any disease or condition that is likely to affect drug metabolism or excretion, at the discretion of the Investigator.
10. History or presence at screening or baseline of cardiac arrhythmia or congenital long QT syndrome.
11. QT interval corrected using Fridericia's formula (QTcF)>450 msec. ECG may be repeated 30 to 60 minutes apart from the first one collected at screening. If repeat ECG is ≤450 msec, the second ECG may be used to determine patient eligibility. However, if repeat ECG confirms QTcF remains >450 msec, the subject is not eligible.
12. Use of tobacco or nicotine containing products in the previous 3 months prior to dosing or a positive urine cotinine test at Screening or Baseline.
13. Lack of willingness to abstain from the consumption of tobacco or nicotine-containing products throughout the duration of the study and until completion of the final Follow-up visit.
14. Regular alcohol consumption defined as ≥21 alcohol units per week (where 1 unit=284 mL of beer, 25 mL of 40% spirit or a 125 mL glass of wine) or the subject is unwilling to abstain from alcohol for 48 h prior to admission and 48 h prior to study visits.
15. Use of any prescription drugs (other than permitted contraception), over-the-counter (OTC) medication, nonsteroidal anti-inflammatory agents (NSAIDs), herbal remedies, supplements or vitamins within the 2 weeks prior to dosing or throughout the duration of the study, without prior approval of the Investigator and written approval of the Medical Monitor.
16. Paracetamol may be utilised, provided that the dose of Paracetamol does not exceed 2 g in any 24 h period.
17. Use of any of the following drugs within 28 days or 10 half-lives of that drug, whichever is longer, prior to study drug administration:
   a. Fluvoxamine, enoxacin, ciprofloxacin;
   b. Other inhibitors of CYP1A2 (including but not limited to methoxsalen or mexiletine);
   c. Inducers of CYP1A2 (such as phenytoin), CYP2C9 or 2C19 (including but not limited to carbamazepine or rifampin);
   d. Any drug associated with substantial risk for prolongation of the QTc interval (including but not limited to moxifloxacin, quinidine, procainamide, amiodarone, sotalol).
18. Vaccination with a live vaccine within the 4 weeks prior to screening or that is planned within 4 weeks of dosing, and any non-live vaccination within the two weeks prior to screening or that is planned within two weeks of dosing (including those for COVID-19).
19. Exposure to any significantly immune suppressing drug within the 3 months prior to screening or 5 half-lives, whichever is longer.
20. Use of any investigational drug or device within 3 months prior to screening.
21. Consumption of grapefruit, grapefruit juice, Seville oranges, Seville orange juice, or any foods containing these ingredients, within 7 days prior to dosing or unwilling to abstain from these throughout the duration of the study.

### Part 3: Patient Cohort

### Inclusion Criteria:

1. Female or male between ≥18 and ≤80 years old (inclusive) at the time of informed consent.
2. At least 6 months and no more than 15 years since the most recent type of surgery related to breast cancer, including sentinel lymph node biopsy, or excision/clearance, axillary lymph node dissection or any type of surgery (excluding fine needle aspiration biopsy [FNA]), at the time of study screening. No intention to have breast reconstructive surgery, nipple reconstruction and/or tattooing during the course of the study.
3. At least 3 months since completion of any neoadjuvant therapy, adjuvant chemotherapy, intravenous and/or oral biologic therapy (e.g., trastuzumab and pertuzumab), radiotherapy, and/or any investigational adjuvant therapy at the time of study screening.
4. At least 3 months since initiation of anti-hormonal oral adjuvant therapy as well as goserelin and/or adjuvant zolendronic acid.
5. Diagnosis of primary breast cancer, and without evidence of local, locoregional and/or distant recurrence and/or metastasis of breast cancer for at least 6 months since breast cancer surgery, as determined at screening and baseline.
6. Documented evidence of pitting oedema in one arm for at least 3 months and also at screening and at least one of the following:
   Increase in relative limb volume of between 10-20% as measured by perometry or the truncated cone method of circumferential tape measurement compared to any prior documented measurement;
   A bioimpedance measure of ≥10 at baseline visit or a change from pre-surgical measure of >+6.5 L-Dex at baseline visit; or
   Overt signs of lymphoedema in the arm clinically indicating Lymphoedema Stage I or II confirmed by asymmetry ≥2 via LymphaScan tissue dielectric constant (TDC) in swell spot.
7. Receiving standard of care compression or agreeable to using care compression, i.e. sleeves and/or pumps and/or manual lymphatic drainage, or no compression care and/or no manual lymphatic drainage ≥4 weeks prior to screening and throughout the study.
8. In good general health at screening and baseline apart from a history of breast cancer and secondary lymphoedema, i.e., free from clinically significant unstable medical, surgical or psychiatric illness (at the discretion of the Investigator); no acute conditions requiring invasive care or hospitalisation; and no conditions or elective procedures requiring invasive intervention within the next 6 months.
9. Vital signs (measured in supine position after 10-minutes rest) at screening:
   a. Systolic blood pressure ≥90 and ≤140 mmHg;
   b. Diastolic blood pressure ≥50 and ≤90 mmHg;
   c. Heart rate ≥45 and ≤100 bpm;
   d. Vital signs may be repeated once, within a minimum of 10 minutes of the completion of the last set of vital signs (while maintaining supine positions until the repeated set of vital signs are collected), if it is suspected that falsely high or low levels have been obtained.
10. Body Mass Index≥18 and ≤35 kg/m² at screening.
11. Willing and able to abstain from direct whole body sun exposure from 2 days prior to dosing and until final study procedures have been conducted. Patients should be instructed to avoid or minimize exposure to sunlight (including sunlamps), use an SPF 50 sun block, or higher, wear clothing that protects against sun exposure and avoid concomitant medications known to cause photosensitivity.

### Part 3: Patient Cohort

### Exclusion Criteria:

1. Bilateral lymphoedema or history of bilateral axillary lymph node removal (i.e., sentinel lymph node biopsy or axillary lymph node dissection), or primary lymphoedema or lymphatic or vascular malformation, determined at screening.
2. Chronic administration (defined as more than 14 consecutive days) of immunosuppressants or other immune-modifying drugs within 3 months prior to study drug administration; corticosteroids are permitted at the discretion of the PI.
3. Recent history (in the 8 weeks prior to screening) of cellulitis, lymphangitis, dermatitis, necrotising fasciitis, or current open wounds or sores in the affected extremity.
4. Fibrotic stranding on affected arm not related to BCRL; history of breast or arm procedures unrelated to axillary node dissection such as non-cancer related reconstructive or cosmetic breast surgery, Botox for hyperhidrosis, chronic intravenous use, port, pic-line, etc., for medical or recreational reasons, tattoos (excluding pink ribbon tattoo designated to inform health caretaker not to take blood pressure in affected arm), or other extreme body modifications, determined at screening.
5. Relative limb volume >20%, stage III secondary lymphedema, or history of clinically diagnosed secondary lymphoedema greater than 4 years, determined at screening.
6. Initiated use of compression or manual lymphatic drainage or other lymphoedema therapies at the start of the study within 4 weeks of the screening visit. Rescreening is allowed following a course of stable compression regimen of >4 weeks.
7. Presence of malignancy, with the exception of adequately treated localised skin cancer (basal cell or squamous cell carcinoma), carcinoma in-situ of the cervix, or unilateral breast cancer history with completed treatment and with no active cancer at the time of screening or in the preceding 6 months.
8. Evidence of clinically relevant medical history/illness at screening, as determined by the Investigator including stroke, uncontrolled hypertension, and other cardiac disease, vascular disease, pulmonary disease, gastrointestinal disease, hepatic disease, renal failure and other kidney disease, rheumatologic disease, coagulopathy or other haematological disease, uncontrolled diabetes and other endocrine disorders, any progressive neurologic disorder, psychiatric disease, dermatological disorder, or surgical history except for orthopaedic and reconstructive breast cancer surgery.
9. Clinically significant infection within 28 days of the start of dosing, as determined by the Investigator.
10. Clinically significant surgical procedure/s, including but not limited to breast cancer reconstruction surgery, within 3 months of screening, or further breast cancer reconstruction surgery planned during the Study.
11. For baseline liver function tests (LFT) 2.5× upper normal limit (UNL) or severe hepatic impairment.
12. Positive test for HCV, HBsAg, or HIV antibody at screening.
13. Currently suffering from clinically significant systemic allergic disease at screening or baseline or has a history of significant drug allergies including a history of anaphylactic reaction (particularly reactions to general anaesthetic agents); allergic reaction due to any drug which led to significant morbidity; prior allergic reaction to pirfenidone.
14. Symptoms of dysphagia or known difficulty in swallowing capsules, determined at screening.
15. History or presence of cardiac arrhythmia or congenital long QT syndrome determined at screening.
16. QTcF>450 msec demonstrated by two ECGs between 30 and 60 minutes apart at screening.
17. Use of tobacco or nicotine containing products in the previous 30 days prior to dosing or a positive urine cotinine test at Screening or Baseline.
18. Regular alcohol consumption defined as >21 alcohol units per week (where 1 unit=284 mL of beer, 25 mL of 40% spirit or a 125 mL glass of wine), determined at screening.
19. Use of any over-the-counter medication, herbal supplements, or diet aids within 48 h prior to dosing.
20. Treated with immunosuppressive or antifibrotic drugs, anti-tumour necrosis factor, immunotherapy, or investigational drugs at screening or within the preceding 30 days.
21. Use of any of the following drugs within 28 days or 10 half-lives of that drug, whichever is the longer, prior to study drug administration:
   a. Fluvoxamine, enoxacin, ciprofloxacin;
   b. Other inhibitors of CYP1A2 (including but not limited to methoxsalen or mexiletine);
   c. Inducers of CYP1A2 (such as phenytoin), CYP2C9 or 2C19 (including but not limited to carbamazepine or rifampin);
   d. Drugs associated with substantial risk for prolongation of the QTc interval (including but not limited to moxifloxacin, quinidine, procainamide, amiodarone, sotalol).
22. Use of any investigational drug or device within 28 days or 10 half-lives of the drug, whichever is the longer, prior to start of dosing.
23. Any condition (e.g., chronic diarrhoea, inflammatory bowel disease or prior surgery of the gastrointestinal tract) that would interfere with drug absorption or any disease or condition that is likely to affect drug metabolism, or excretion, determined at screening.
24. History of anaphylactic reaction (particularly reactions to general anesthetic agents); allergic reaction due to any drug which led to significant morbidity; prior allergic reaction to pirfenidone.

### Dosage and Mode of Administration:

All subjects in Parts 1 or 2 will be randomised 3:1 to receive either LYT-100 (deupirfenidone) formulated as powder in capsules, or placebo (a matching, inactive capsule containing methyl cellulose). All patients in Part 3 will be randomized to receive LYT-100 or placebo in a 1:1 ratio. The dosing regimen per study part and cohort is presented below:

TABLE 15
Parts 1, 2, and 3: Cohorts and Number of Participants or Patients per Treatment Arm

| Cohort⁺ | | Placebo | | LYT-100 | | LYT-100 dose⁺ |
|---|---|---|---|---|---|---|
| | PART 1 ^{A} | | | | | |
| | (Healthy | | | | | |
| | Volunteers) | | | | | |
| 1 | Multiple Dose | 2 | participants | 6 | participants | 100 mg, BID with |
| | (n = 8) | | | | | food X 5 days |
| 2 | Multiple Dose | 2 | participants | 6 | participants | 250 mg, BID* with |
| | (n = 8) | | | | | food X 5 days |
| 3 | Multiple Dose | 2 | participants ^{A} | 6 | participants ^{A} | 500 mg, BID* with |
| | (n = 8) | | | | | food X 5 days |
| 4 | Multiple Dose | 2 | participants ^{A} | 6 | participants ^{A} | 750 mg, BID* with |
| | (n = 8) | | | | | food X 5 days |
| 6 | Multiple Dose | 2 | participants ^{A} | 6 | participants ^{A} | 1000 mg, BID* with |
| | (n = 8) | | | | | food X 5 days |
| | PART 2 ^{A} | | | | | |
| | (Healthy | | | | | |
| | Volunteers) | | | | | |
| 5† | Fed/Fasted | 2 | participants ^{A} | 6 | participants ^{A} | 500 mg, single dose |
| | Cohort (n = 8) | | | | | fed and fasted** |
| | PART 3 (Patients | | | | | |
| | with Secondary | | | | | |
| | Lymphoedema) | | | | | |
| 7** | Patient Cohort | 25 | participants | 25 | participants | LYT-100 BID |
| | (n = up to 50) | | | | | (titrate to 750 mg)** |
| | | | | | | or matching placebo |
| | | | | | | for 179 days |

⁺For Part 1, the SRC may approve dose escalation to the next dose or to an intermediate dose (a dose lower than the next planned dose). The number of cohorts may be expanded and number of participants within a cohort may be increased.
^{A} All subjects from prior cohorts from Part 1 will be invited to return following a minimum 7-day washout to participate in the Part 2 Fed/Fasted cohort (Cohort 5), where a single dose of 500 mg LYT-100 or Placebo will be administered under fed or fasted conditions. The alternate fast/fed meal sequence for the second single dose received will occur ≥7 days later.
†Cohort 5 will consist of any subjects who participated previously in another of the cohorts in Part 1. A washout period of at least 7 days will apply between single dosing under fed conditions and fasted.
*Dose escalation and adjustment will be dependent on SRC review of all safety and available PK data, with no dose exceeding 1000 mg BID.
**Patients will be administered LYT-100 study medication, or placebo, orally without regard to food with approximately 10 to 12 hours between the two daily doses. Doses may be adjusted according to safety and tolerability to avoid toxicity by adjusting to lower doses in response to patient safety and tolerability issues. If dosing titration is not well tolerated, adjustments to dosing may be made as follows: reductions to 250 mg, BID X 2 days (may be longer if needed), 500 mg BID X 2 days (may be longer if needed), 750 mg BID thereafter vs. matching placebo. In addition, if tolerability issues persist, the patient may be instructed to take study medication with food.

Duration of Treatment with Study Medication:

Part 1: 5 days

Part 2: 2 single dose days

Part 3: 26 weeks. Note that patients will each have a screening and post-treatment follow-up period.

### Criteria for Evaluation:

### Safety:

Safety and tolerability will be assessed throughout Part 1, Part 2 and Part 3 of the study by monitoring AEs, physical examination, vital signs, 12-lead ECGs, clinical laboratory values (haematology panel, multiphasic chemistry panel and urinalysis), and review of concomitant treatments/medication use.

### Efficacy:

For Part 3 only, the following parameters will be measured at each study visit except the screening visit:
Limb water content, by Bioelectrical impedance spectroscopy: Multiple frequency bioelectrical impedance spectroscopy (L-dex) provides accurate relative measures of protein-rich fluid in the upper limb of patients. BIS is a non-invasive technique that involves passing an extremely small electrical current through the body and measuring the impedance (or resistance) to the flow of this current. The electrical current is primarily conducted by the water containing fluids in the body. BIS quantifies the amount of protein-rich fluid in lymphoedema by comparison of the affected to the non-affected limb.
Limb volume (truncated cone tape measure and/or perometry). Perometry is a non-invasive technique involving a Perometer (Pero-System), which uses infrared light to scan a limb and obtain measurements of the limb's circumference.
Tissue dielectric constant (MoistureMeterD): The tissue dielectric constant measures the local tissue water content under the skin at various depths ranging from skin to subcutis. The results are converted into a 0-100% scale to reflect subcutaneous fluid deposition that can occur in early stage lymphoedema.
Tissue firmness (tonometry/SkinFibroMeter): A tonometer device is pressed into the skin to measure the amount of force required to make an indent in the tissue. The resulting measurement gauges the degree of firmness or fibrosis (tissue scarring) under the skin to assess the severity of lymphoedema. (r) at dorsal surface of arm 10 cm below the elbow
Visual-analogue scales for pain, swelling, discomfort, and function: This graphic scale has a straight line with endpoints of 0 and 10 that is marked by the patient to calibrate to their extreme limits of pain, swelling, discomfort and function, ranging from “not at all” to “as bad as it could be”. The higher marks on the line indicates the worse condition.

### Health-related Quality of Life (QoL):

For Part 3 only, the following will be assessed at Day −1, Weeks 1, 12 and 26 (or early termination):
Lymphedema Symptom Intensity and Distress Survey-Arm (L-SIDS); This is a self-report tool consisting of 36 items to evaluate arm lymphoedema and associated symptoms in breast cancer survivors. Symptoms are rated on a ten-point scale (5 points for intensity of the symptom and 5 point for how distressed the patient felt) for heaviness, tightness, pain, stabbing pain, cramping, numbness, achiness, swelling, hardness, tingling, pins and needles, difficulty moving, raising the arm and sadness. Lower scores indicate a higher quality of life.
Lymphoedema Quality of Life Tool, Arm (LYMQOL). This is a patient completed questionnaire that assesses upper limb lymphoedema and symptoms and ability to perform common functional activities in patients with BCRL. It addresses the following four domains—Symptoms, Body Image/Appearance, Function and Mood. Each item is scored as 1=Not at all; 2=A little; 3=Quite a bit; and 4=A lot. Total scores for each domain are summed and divided by the total number of completed question responses. The overall QOL item ranges from 1-10. Lower scores indicate a higher quality of life.

### Occurrence of Infection:

For Part 3 only, the following will be assessed along with Adverse Events:
Cellulitis
Lymphangitis

### Pharmacokinetics:

Participants will provide blood samples at Baseline (Day −1) for the determination of CYP1A2, CYP2C9, CYP2C19, and CYP2D6 genotype to support exploratory PK analyses. Participants are required to provide consent for genotyping.

### Parts 1 and 2 (Healthy Subjects)

In Part 1, blood samples (4 mL each) for PK will be collected for Cohorts 1, 2, 3, 4, and 6 at specified times (hours) at each as follows:
Day 1 and Day 5: 0 (pre-dose), 0.5, 1, 1.5, 2, 2.5, 3, 3.5, 4, 6, 8, 12 (pre-dose), 13, 14, 15, 16, 18 h
Day 2, Day 3 and Day 4: 0 (pre-morning-dose) and 12 h (pre-evening-dose)
Day 6: 12, 18, 24 and 36 h post-last dose
Day 7: 48 h (post-last dose)

Data will be used to assess the PK profiles of multiple BID doses of LYT-100 administered with food over 5 days for dose proportionality.

In Part 2, blood samples (4 mL each) for PK will be collected for Cohort 5 fed and fasted at specified times as follows:
Day 1: 0 (pre-dose), 0.5, 1, 1.5, 2, 2.5, 3, 3.5, 4, 6, 8, 10, 12, 15, 18 h
Day 2: 24 h post-dose
Day 3: 48 h post-dose

Data will be used to descriptively compare the PK profile and compare the relative bioavailability of a single dose of LYT-100 administered at a dose below the MTD with food, to the equivalent dose without food. Comparison of the PK parameters in the fed and fasted states will be performed, and analysis of PK by gender may be performed if the data allow.

Plasma concentration time data for LYT-100, and its metabolite(s) will be analysed using non-compartmental methods. Pharmacokinetics for Day 1 to 3 in Part 2 (fed state) will be compared to PK for Day 1 to Day 3 in Part 2 (fasted state). Plasma PK parameters (non-compartmental or compartmental analysis as appropriate) will include, but are not limited to:
Following single dose: Tₘₐₓ, t_{lag}, Cₘₐₓ, AUC₀₋₄, AUC₀₋ₗₐₛₜ, AUC_{0-inf}, lambda z, % AUCₑₓₜ, CL/F, Vz/F, Cₘₐₓ/D, AUC₀₋ₗₐₛₜ/D, AUC_{0-inf}/D and MRT (D is dose)
After repeated doses: C_{trough}, C_{trough}/D
Following repeated doses on last day: Tₘₐₓ, Cₘₐₓ, C_{avg}, t_{lag}, t_{1/2}, AUC₀₋₄, AUC₀₋ₗₐₛₜ, AUC_{0-inf}, lambda z, % AUCₑₓₜ, CLss/F, Vzss/F, MRT, PTF, Rac(AUC), Rac(Cₘₐₓ), Cₘₐₓ/D, AUCₗₐₛₜ/D, AUC_{0-inf}/D and Rac (accumulation ratio)
   Part 3 (Patients with Secondary Lymphoedema Post-Axillary/Post-Sentinel Node Dissection)

Blood samples (4 mL each) for population pharmacokinetics will be collected for Cohort 7 at specified times at any visit from Week 1 to Week 26 of the study. Each patient will provide up to a minimum of 1 sample at each timepoint in reference to dosing: Pre-dose, 1 to <2 h, 2 to ≤4 h, 4 to ≤8 h, 8 to 12 h.

Sparse PK sampling will be employed for population PK analysis (as a secondary endpoint) to determine the variability of LYT-100 drug concentration data from individual patients across multiple clinical sites.

The conclusion from Part 2 demonstrated that the food effect on the PK Cₘₐₓ of LYT-100 appears to be less than the reported food effect PK Cₘₐₓ of pirfenidone, which is thought to be related to the acute adverse events of pirfenidone. There were no clear correlations between the adverse events seen and the fed and fasted states. The overall safety and tolerability profile with and without food, taken together with the reduced food effect on Cₘₐₓ in Part 2 of this study suggests that there is no clear rationale for patients to take LYT-100 with regard to food. If safety and tolerability issues are reported during the study, the patient may be instructed to take study medication with food during Part 3.

### Fibrotic and Inflammatory Biomarkers:

The following parameters will be measured in Part 3 only at all study visits except the screening visit:
Fibrotic and inflammatory biomarkers (G-CSF, MIG, FGF-2, IL-4, IL-10, lymphotoxin-α/TNF-β, leptin, IL-6, IL-1β, TNF-α, TGF-β1, MMP-9, TIMP-1, MCP-1).

These data may be reported separately in a supplementary report to the main Clinical Study Report.

### Study Endpoints for the Study are Defined as Follows:

### Part 1 and Part 2

Safety and tolerability (AEs, physical examination, vital signs, ECGs, clinical laboratory parameters [haematology panel, multiphasic chemistry panel and urinalysis], and concomitant treatments).
Pharmacokinetic parameters (AUC₍₀₋₁₂₎, AUC₍₀₋₂₄₎, t_{1/2}, VZ/F, CL/F, Cₘₐₓ, C₂₄, Cₘᵢₙ (T₂₄), and Tₘₐₓ).

### Part 3

### Primary Endpoint:

Safety and tolerability (AEs, physical examination, vital signs, ECGs, clinical laboratory parameters [haematology panel, multiphasic chemistry panel and urinalysis], and concomitant treatments).

### Secondary Endpoint:

Efficacy:
   Limb water content, by BIS.
   Limb volume (perometry).
   Tissue dielectric constant (MoistureMeterD).
   Tissue firmness (tonometry/SkinFibroMeter).
   Visual-analogue scales for pain, swelling, discomfort, and function.
Infection
   Cellulitis
   Lymphangitis
Health-related QoL:
   Lymphedema Symptom Intensity and Distress Survey-Arm (L-SIDS)
   Lymphoedema Quality of Life Tool (LYMQOL)
Population PK parameters.
Fibrotic and Inflammatory biomarkers:
   Fibrotic and inflammatory biomarkers (G-CSF, MIG, FGF-2, IL-4, IL-10, lymphotoxin-α/TNF-β, leptin, IL-6, IL-1β, TNF-α, TGF-β1, MMP-9, TIMP-1, MCP-1).

Comparisons between LYT-100 and placebo will be based on clinical interpretation of effect, magnitudes of effect, and a preponderance of evidence. Estimates of changes over time from these data may be used to power future clinical studies. The number of occurrences of cellulitis and lymphangitis within the treatment period will be tabulated. Visual-analogue scales (VAS) for pain, swelling, discomfort, and function, and QoL assessments using the LSIDS-A and LYMQOL questionnaires will be provided at baseline and treatment period timepoints.

### Statistical Methods

In Part 1 and Part 2, eight subjects per cohort were chosen to adequately characterise the rate and extent of absorption as measured by select PK parameters, and to allow comparison of PK in a fed versus fasted state.

Part 3 will randomize 50 patients with secondary lymphoedema, in a 1:1 ratio to LYT-100 or placebo, as part of this early development and exploratory study of LYT-100. Formal sample size calculations will not be performed; rather, the sample size selected should be adequate for preliminary evaluation of safety, tolerability, efficacy signalling, PK and fibrotic and inflammatory biomarker parameters in the targeted patient population.

### General Statistical Plan

The analysis will be consistent with the study design, with assessment of each Part performed separately. The baseline for all variables will be the last measurement obtained prior to the participant receiving the first dose of study treatment.

Participant disposition (including the number and percent of participants/patients who are enrolled, who receive treatment, who prematurely discontinue and reasons for discontinuation, and who complete the study) will be tabulated by treatment group. Summary statistics for days of exposure and concentration of exposure will be provided by treatment group.

Adverse events, concomitant medications, clinical laboratory findings, physical examinations, ECGs and vital signs for each participant will be tabulated or summarised descriptively, where appropriate.

Demographic information will be presented for each participant and summarised. Treatment-emergent adverse events and laboratory, vital signs, and ECG parameters will be summarised. In addition, change from baseline will be summarised for laboratory and vital sign parameters. Shift tables will also be provided for clinical laboratory results. ECG results will be classified as normal and abnormal and summarised. ECG results for QTcF will also be classified as <450 msec, 450-500 msec or >500 msec. Changes in physical exams will be described.

### Study Populations

Analysis populations will be defined for each Part separately. In general:
The Safety population is defined as all participants who receive LYT-100 or Placebo.
The Full Analysis Set (FAS) population is defined as all randomized participants who received at least one dose of LYT-100 and had at least one post-baseline efficacy assessment. All efficacy endpoints will be assessed using this population.
The Pharmacokinetic Population (PP) is defined as all participants who receive LYT-100 and for whom an evaluable concentration-time profile is available for the determination of at least one PK variable.
The Fibrotic and Inflammatory Biomarker Population is defined as all patients in Part 3 only who receive LYT-100 and who provide biomarker and lymphoedema assessment data.

### Analysis of PK in Parts 1 and 2

Analyses will be performed for each cohort separately. Determination of steady state for LYT-100 will be performed using Helmert Contrasts using trough concentrations at Days 2, 3, 4, 5, and 6. Pharmacokinetic parameter values will be listed and summarised by dose. Dose-linearity across the LYT-100 doses will be assessed. Comparison of the PK parameters in the fed and fasted states will be performed, and analysis of PK by gender may be performed if the data allow.

### Part 3

Analysis of clinical assessments and potential progression or disease will be explored. Comparisons between LYT-100 and placebo will be based on clinical interpretation of effect, magnitudes of effect, and a preponderance of evidence. Estimates of changes over time from these data may be used to power future clinical studies.

The primary endpoints are safety (clinical laboratory parameters, vital signs, ECGs and spontaneously reported AEs) and tolerability. Secondary endpoints include efficacy (lymphoedema assessments, infection and health related QOL), population PK and fibrotic and Inflammatory biomarkers: Change from Baseline to each post-Baseline visit (through Week 26; on-treatment effect), as well as the change from Baseline to Week 28, 36 and Week 48 on the following endpoints will be calculated for the following endpoints: fibrotic and inflammatory biomarkers (G-CSF, MIG, FGF-2, IL-4, IL-10, lymphotoxin α/TNF-β, leptin, IL-6, IL-1(3, TNF-a, TGF-β1, MMP-9, TIMP-1, MCP-1), limb water content (BIS), limb volume (truncated cone tape measure and/or perometry), tissue dielectric constant (MoistureMeterD), tissue firmness (tonometry/SkinFibroMeter), visual-analogue scales (pain, swelling, discomfort, and function), and QoL (LSIDS-A, LYMQOL). For select efficacy outcomes, use of a mixed model for repeated measures (MMRM) will be used to provide model-based estimates of changes over time in each of these outcomes. Descriptive statistics will also be provided at each time point. Details for the model (including covariates to be included) will be provided in the SAP. Data for all endpoints will be tabulated, displayed graphically or summarised descriptively, as appropriate. No formal hypothesis testing will be performed.

### Results for Study Part 1

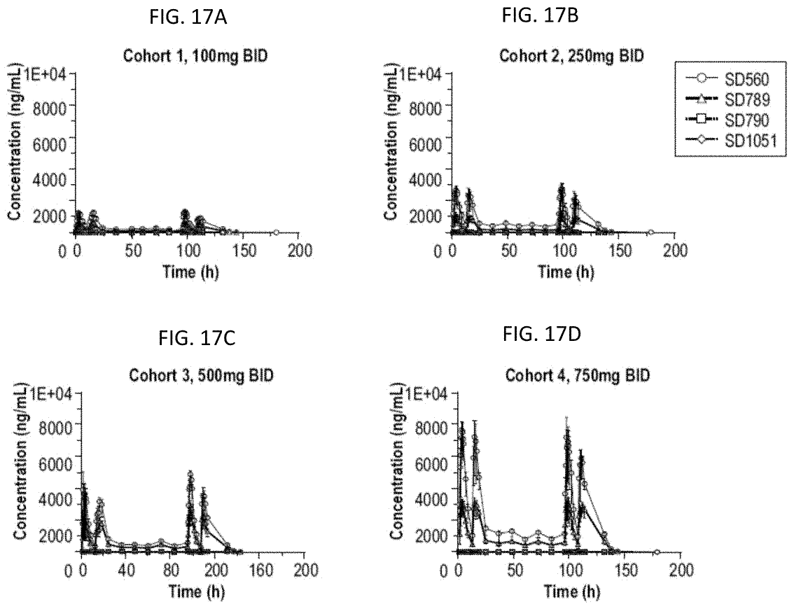

All cohorts (n=6 each) were dosed every 12 hours with food for 5 days at either 100, 250, 500, or 750 mg BID. FIGS. 17A-17D summarize the pharmacokinetics over 7 days (48 hours after last administration) for the active LYT-100 (deupirfenidone; SD-560) and the major metabolites, d2-5-hydroxymethyl-pirfenidone (LYT-110; SD-790), nondeuterated 5-carboxy-pirfenidone (LYT-105; SD-789), and d3-4′-hydroxy-pirfenidone (SD-1051). FIG. 17A shows the concentration in ng/mL of the active and each metabolite at each timepoint for the 100 mg BID Cohort 1. FIG. 17B shows the concentration in ng/mL of the active and each metabolite at each timepoint for the 250 mg BID Cohort 2. FIG. 17C shows the concentration in ng/mL of the active and each metabolite at each timepoint for the 500 mg BID Cohort 3. FIG. 17D shows the concentration in ng/mL of the active and each metabolite at each timepoint for the 750 mg BID Cohort 4. In each case, the data demonstrated increased exposure to drug and metabolites with increasing dose.

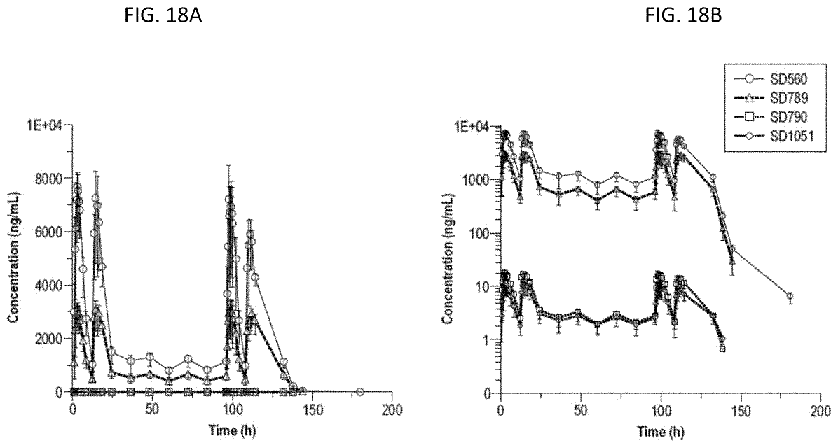

FIGS. 18A and 18B (log scale) provide the concentration in ng/mL of the active and each metabolite at each timepoint for the 750 mg BID Cohort 4. The exposure of LYT-100 (SD-560) was greatest, followed by 5-carboxy-pirfenidone (SD-789). Exposures for d2-5-hydroxymethyl-pirfenidone (SD-790) and d3-4′-hydroxy-pirfenidone (SD-1051) were approximately equal to each other, and more than two orders of magnitude less than that of the parent or SD-789, consistent with previous cohorts. Cₘₐₓ was similar to previous cohorts, and C_{trough} value was roughly constant, and no obvious accumulation was observed.

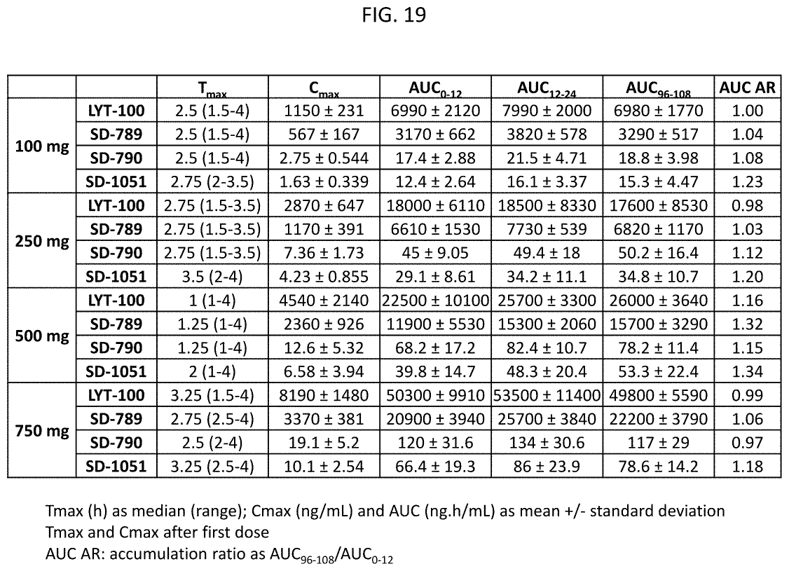

Pharmacokinetic data (Tₘₐₓ, Cₘₐₓ, AUC₀₋₁₂, AUC₁₂₋₂₄, AUC₉₆₋₁₀₈, and AUC accumulation ratio (AUC₉₆₋₁₀₈/AUC₀₋₁₂)) for each dosing Cohort (100 mg, 250 mg, 500 mg, and 750 mg BID) for the active and each metabolite (SD-789, SD-790, and SD-1051) is provided in FIG. 19. The data demonstrate that no major accumulation of LYT-100 or its metabolites occurred over the length of the study, with similar accumulation ratios (˜1) occurring across all dose groups. The data also confirmed that SD-789 was the major metabolite, and had an average ratio to the parent (M/P) by AUC of 0.45 (0.37-0.50). The minor metabolites SD-790 and SD-1051 had M/P of 0.0025 (0.0020-0.0029) and 0.0017 (0.0012-0.0022), respectively.

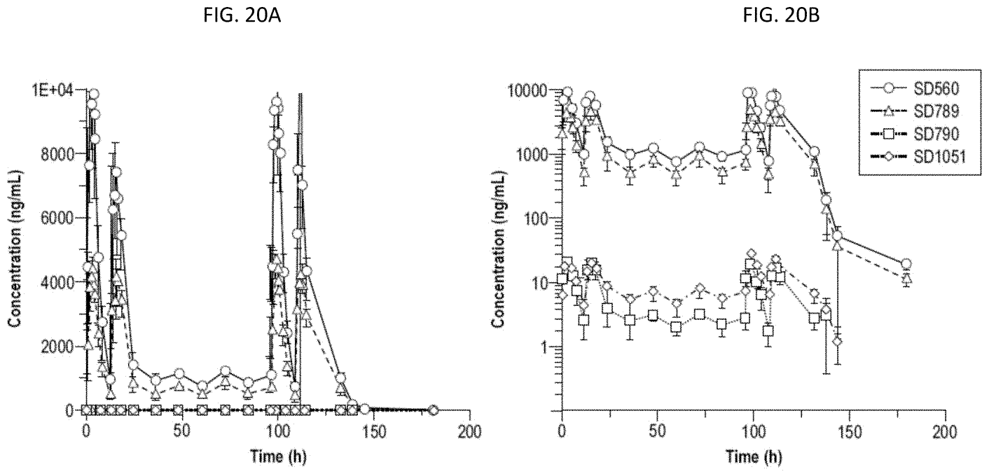

Pharmacokinetic data for LYT-100 and each metabolite (SD-789, SD-790, and SD-1051) are provided for Cohort 6 (1000 mg BID) in FIGS. 20A and 20B (linear and log scale concentration axes, respectively), which demonstrated similar results as for previous cohorts. The exposure of LYT-100 (SD-560) was greatest, followed by 5-carboxy-pirfenidone (SD-789). Cₘₐₓ was similar to previous cohorts, and C_{trough} value was roughly constant, and no obvious accumulation was observed.

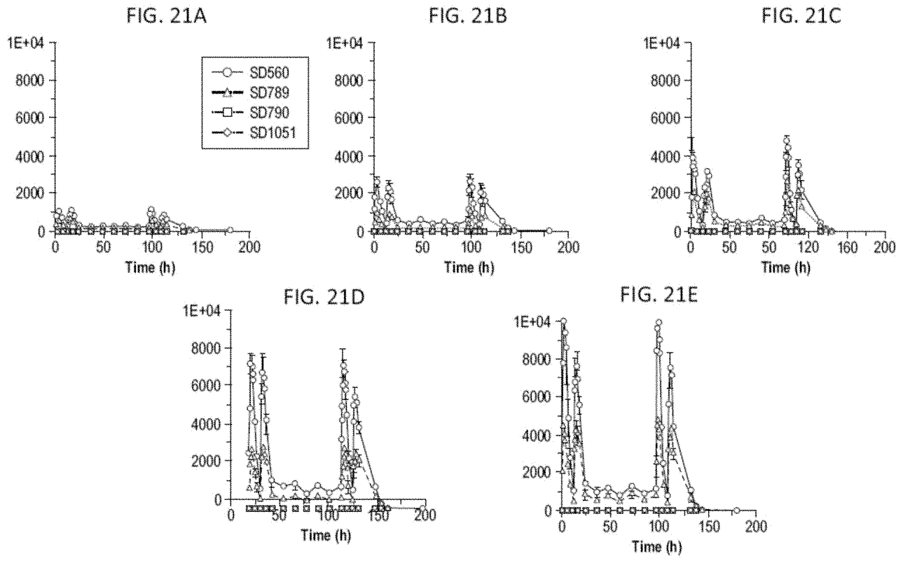

Pharmacokinetic data for LYT-100 and each metabolite (SD-789, SD-790, and SD-1051) are provided for each dosing Cohort (100 mg, 250 mg, 500 mg, 750 mg, and 1000 mg BID) in FIGS. 21A-21E, respectively, which demonstrated increased exposure to drug and metabolites with dose.

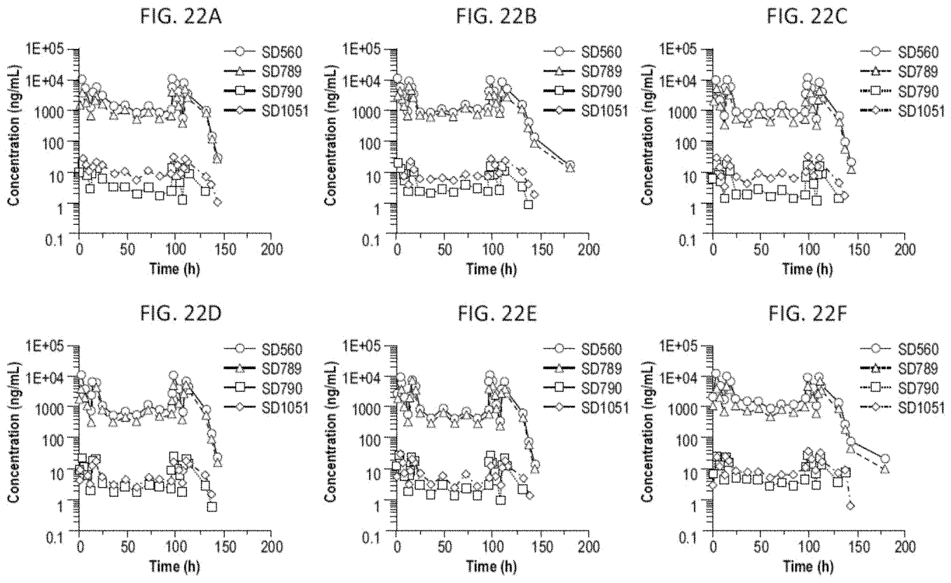

Pharmacokinetic data for LYT-100 and each metabolite (SD-789, SD-790, and SD-1051) are provided for each of the six subjects of Cohort 6 (1000 mg BID) in FIGS. 22A-22F, which demonstrated little variation across subjects.

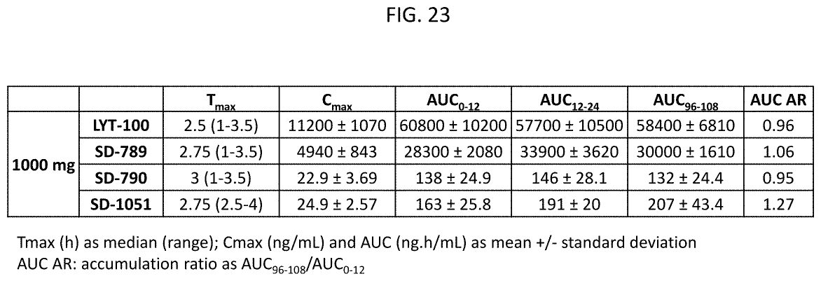

Pharmacokinetic data for LYT-100 and each metabolite (SD-789, SD-790, and SD-1051) are provided for each dosing Cohort (100 mg, 250 mg, 500 mg, 750 mg, and 1000 mg BID) in FIG. 23. The data demonstrate that no major accumulation of LYT-100 (accumulation ratios (˜1) across all dose groups) or its metabolites occurred over the length of the study. The data also confirmed that SD-789 was the major metabolite, and had an average ratio to the parent (M/P) by AUC of 0.56 (0.44-0.66). The minor metabolites SD-790 and SD-1051 had M/P of 0.0028 (0.0018-0.0038) and 0.0037 (0.0030-0.0058), respectively.

The dose dependent AUC was evaluated for LYT-100 and SD-789 across all dose Cohorts using the AUC₉₆₋₁₀₈ data points. The data (FIG. 24) demonstrated linear dose proportionality for both parent and major metabolite.

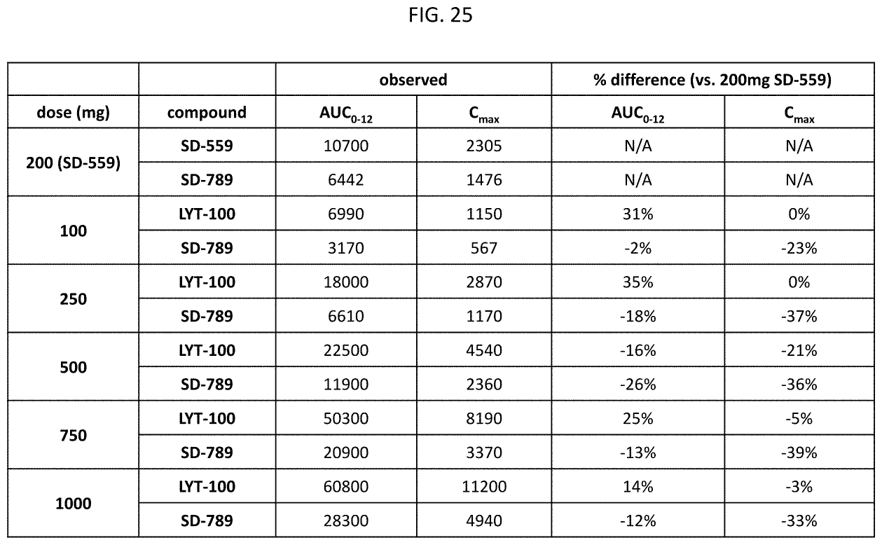

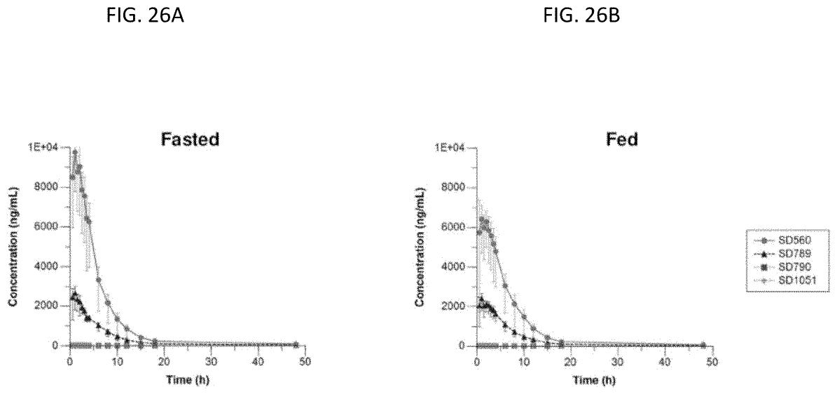

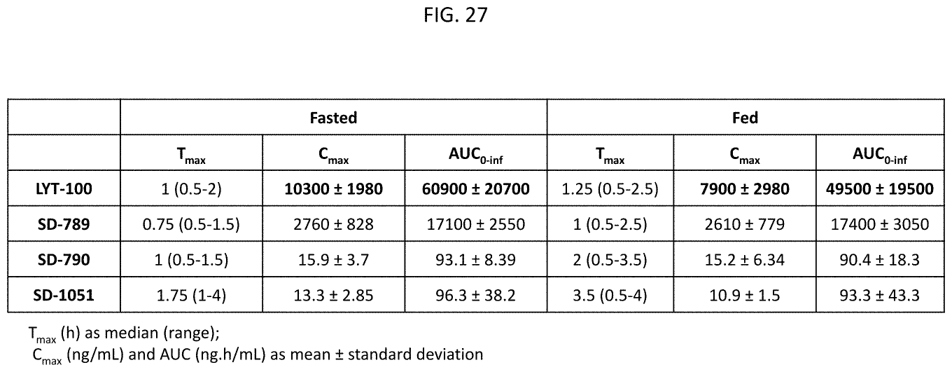

Data across the Cohorts was compared against data from Huang et al. at 200 mg BID pirfenidone, extrapolated to 100, 250, 500, 750, 1000 mg assuming dose proportionality, and comparing to AUC₀₋₁₂ and Cₘₐₓ, LYT-100 and SD-789 only. With the exception of the 500 mg dose, there was an increase for LYT-100 AUC over that of SD-559 and a decrease for SD-789 Cₘₐₓ over that of SD-559 (FIG. 25). Pharmacokinetic data for LYT-100 and each metabolite (SD-789, SD-790, and SD-1051) are provided for dosing Cohort 5 (500 mg single dose; fasted and fed states) in FIGS. 26A and 26B (fasted and fed, respectively) which demonstrated a similar exposure profile as in the other Cohorts, but with a markedly lower fed Cₘₐₓ. Pharmacokinetic data for Cohort 5 (Tₘₐₓ, Cₘₐₓ, AUC_{0-inf}) for the active and each metabolite in fasted and fed subjects following a single 500 mg dose is provided in FIG. 27. The data demonstrated that in the fed state, the Cₘₐₓ and AUC of LYT-100 were decreased relative to that achieved when subjects were dosed in the fasted state (23% and 19% decrease in Cₘₐₓ and AUC, respectively). Pirfenidone demonstrates a much greater food effect on Cₘₐₓ (49% decrease in fed state) For LYT-100, there was no food effect on Cₘₐₓ or AUC of LYT-100 metabolites. In contrast to food effects on pirfenidone (median Tₘₐₓ shift from 0.5 to 3), there was no food effect on the Tₘₐₓ of either LYT-100 or its metabolites.

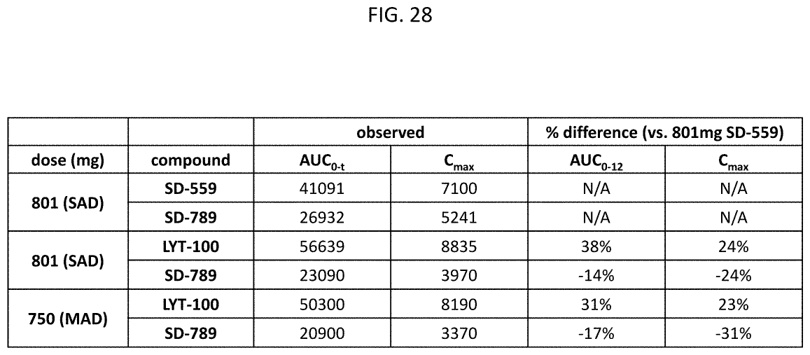

A comparison of data from the 750 mg MAD dose, pirfenidone at 750 mg, and the SAD dose of LYT-100 extrapolated to 750 mg (FIG. 28). The 750 mg MAD data reiterated observations in the SAD study, and demonstrated that there was higher exposure to LYT-100 than SD-559 for the same dose, and lower exposure to SD-789 after LYT-100 vs. SD-559 dosing at same dose level. This data confirms the deuterium kinetic isotope effect for LYT-100 vs. SD-559 on metabolic profile.

### Adverse Event Profile

Based on the blinded data obtained in the MAD study, all adverse events were mild and transient. The most commen events were headache and mild abdominal discomfort. Further, in contrast to pirfenidone (Phase I study, PIPF-005), LYT-100 did not demonstrate a dose-related increase in adverse events based on the MAD blinded study data. For example, the low 250 mg BID dose had the most AEs, while the high dose (1000 mg) had the lowest (two incidences of headache). There were no dose limiting toxicities observed, and no maximally tolerated dose was reached.

### Example 3: LYT-100 Significantly Reduced Area of Fibrosis in Mouse Model

Non-alcoholic steatohepatitis (NASH) is characterized by lobular inflammation, hepatocyte ballooning and degeneration progressing to liver fibrosis. LYT-100 was orally administered at 0 mL/kg (Vehicle only: 0.5% CMC) or 10 mL/kg twice daily from 6-9 weeks of age in 18 male mice in which NASH mice was induced by a single subcutaneous injection of 200 streptozotocin solution 2 days after birth and feet with a high fat diet after 4 weeks of age. LYT-100 was administered at an oral dose of 30 mg/kg twice daily (60 mg/kg/day). In addition, nine non-NASH mice were fed with a normal diet and monitored.

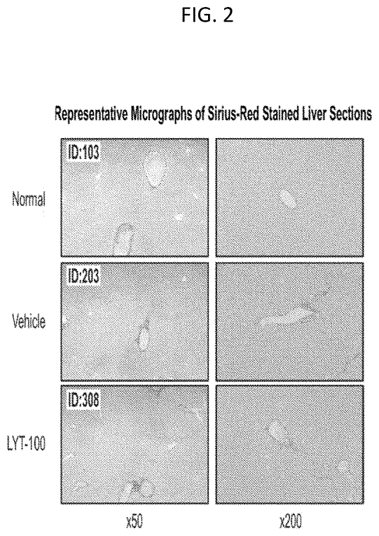

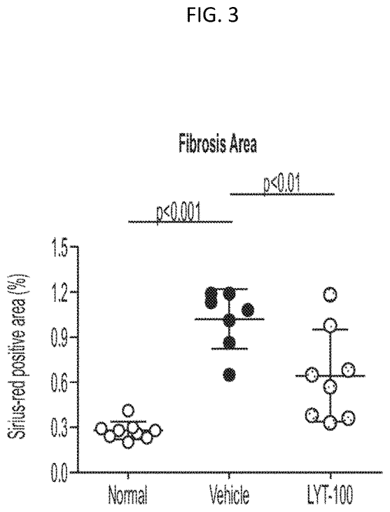

FIG. 2 depicts representative micrographs of Sirius-red stained liver sections illustrating that LYT-100 significantly reduced the area of fibrosis. Specifically, liver sections from the vehicle group exhibited collagen deposition in the pericentral region of the liver lobule. And the LYT-100 group showed a significant reduction in the fibrosis area compared to the vehicle group. These results demonstrate that LYT-100 has a potential to inhibit the progression of fibrosis. FIG. 3 illustrates the percent fibrosis area for LYT-100 versus vehicle and control. The results are also summarized Table 16 below.

TABLE 16
Fibrosis Area

| Parameter | Normal | Vehicle | LYT-100 |
|---|---|---|---|
| (mean ± SD) | (n = 9) | (n = 7) | (n = 8) |
| Fibrosis Area (%) | 0.27 ± 0.06 | 1.02 ± 0.20 | 0.64 ± 0.31* |

*p < 0.01, Vehicle vs LYT-100

Liver sections from the Vehicle group exhibited severe micro- and macro vesicular fat deposition, hepatocellular ballooning and inflammatory cell infiltration. While LYT-100 hepatocyte ballooning was similar to Vehicle, scores were lower for lobular inflammation and steatosis. (Table 17).

TABLE 17
NAFLD Activity Score

| | | Score | | | | | | | | | | | |
|---|---|---|---|---|---|---|---|---|---|---|---|---|---|
| | | Steatosis | | | | Lobular Inflammation | | | | Hepatocyte ballooning | | | NAS |
| Group | n | 0 | 1 | 2 | 3 | 0 | 1 | 2 | 3 | 0 | 1 | 2 | (mean ± SD) |
| Normal | 9 | 9 | — | — | — | 9 | — | — | — | 9 | — | — | 0.0 ± 0.0 |
| Vehicle | 7 | 2 | 5 | — | — | — | 2 | 1 | 4 | — | 1 | 6 | 4.9 ± 1.2 |
| LYT-100 | 8 | 4 | 3 | 1 | — | — | 5 | 3 | — | — | — | 8 | 4.0 ± 1.1 |

Definition of NAS Components

| Item | Score | Extent |
|---|---|---|
| Steatosis | 0 | <5% |
| | 1 | 5-33% |
| | 2 | >33%-66% |
| | 3 | >66% |
| Hepatocyte | 0 | None |
| Ballooning | 1 | Few balloon cells |
| | 2 | Many cells/prominent ballooning |
| Lobular | 0 | No foci |
| Inflammation | 1 | <2 foci/200x |
| | 2 | 2-4 foci/200x |
| | 3 | >4 foci/200x |

As evidenced above, LYT-100 significantly reduced the area of fibrosis, reduced inflammation, and reduced accumulation of fat (steatosis), as compared to the untreated NASH mice.

### Example 4: LYT-100 Reduction of TGF-β-Induced Proliferation and Collagen Levels in Primary Mouse Lung Fibroblasts

LYT-100 was evaluated for an ability to reduce the TGF-β-induced proliferation of, and collagen levels in, Primary Mouse Lung Fibroblasts (PMLF).

Inhibition of p38 members by LYT-100 is important as p38 members are activated by TGF-β signaling pathway. TGF-β activation, in turn plays a significant role in transcriptional induction of the collagen type IA2. The collagen type IA2 makes up the majority of extracellular matrix, which accumulates during progression of, e.g., IPF. Deposition of collagen is one of the most important components of fibrotic lung tissue, a process primarily induced by TGF-β. Since accumulation of insoluble collagen encroaches on the alveolar space, it plays pivotal role in distortion of lung architecture and progression of IPF. Therefore, inhibition of TGF-β-induced collagen synthesis is an important target for IPF. In addition to insoluble (structural) collagen, fibrotic lungs of IPF patients also show high levels of non-structural (soluble) collagen.

Although this type of collagen may eventually become insoluble collagen, until then, soluble collagen can serve as a ligand for integrin receptors of lung fibroblasts and epithelial cells. Binding of soluble collagen to these receptors induces proliferation and migration of these cells. Fibronectin is another important component of fibrotic lungs as it is induced by TGF-β and functions both as a structural component of extra cellular matrix (ECM), as well as a ligand for integrin receptors. Just like soluble collagen, binding of fibronectin to integrin receptors induces the proliferation of fibroblast and epithelial cells of the lungs and plays significant role in progression of IPF.

### Preparation of Primary Mouse Lung Fibroblast

Primary Mouse lung fibroblast were prepared as follows. One lung was removed from 2 months old male BalbC Mouse, perfused with sterile PBS, minced and incubated in 2 ml of serum free Dulbecco's Modified Eagle's Medium (DMEM) containing 100 μg/ml of collagenase I for one hour at 37° C. Each sample was centrifuged at 1500 r.p.m (revolution per minute) for 5 minutes, washed three times with PBS and the final cellular pellet was resuspended in DMEM supplemented with 10% serum and Pen/Strep, and incubated in 150 mm plates at 37° C. with 80% humidity and %% CO2. The growth medium was removed and fresh medium was added every day for 10 days.

### Testing the Effect of LYT-100 on Survival of Primary Mouse Lung Fibroblast

LYT-100 was evaluated for an ability to alter TGF-β-induced proliferation of PMLF. At the end of 10-day incubation period above, lung fibroblasts were confluent. Before testing the effect of LYT-100 on survival of these cells, fibroblasts were tripsinized and five thousand cells were plated into 96 well plate in 200 μL complete DMEM, and incubated until cells reached to 95-100% confluency, then the medium was removed and complete DMEM containing Prolin (10 μM) and Ascorbic acid (20 μg/ml) was added. LYT-100, dissolved in pure ethanol, was added to the plates at a final concentration of 500 μM 1 h prior to addition of TGF-β (5 ng/ml), and cells were further incubated for 72 hrs. One hundred μL of the growth medium was removed and 20 μL of MTT stock solution (prepared in PBS at 5.5 mg/ml concentration) was added and cells were incubated for 4 hrs, then 100 μl of DMSO was added, and absorbance of developed color was monitored at 540-690 nm.

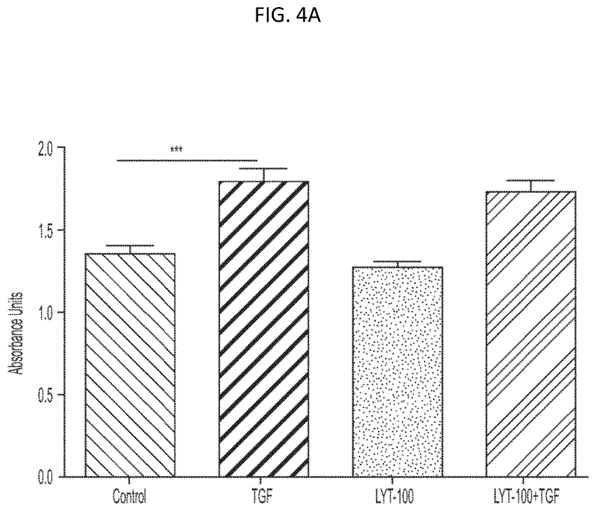

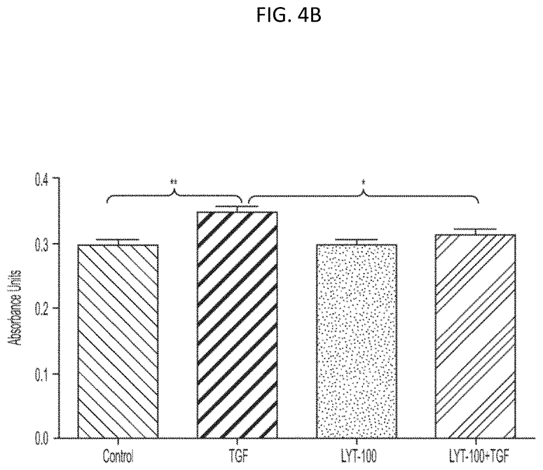

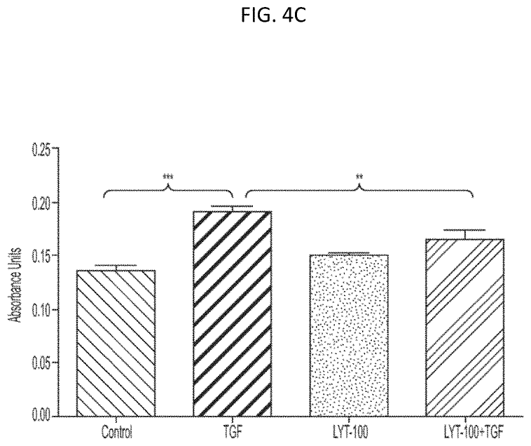

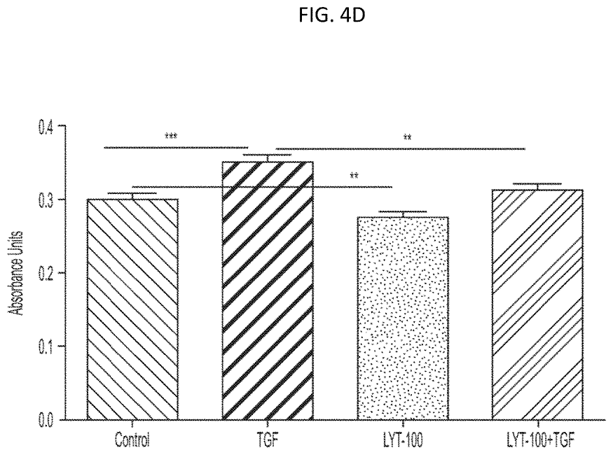

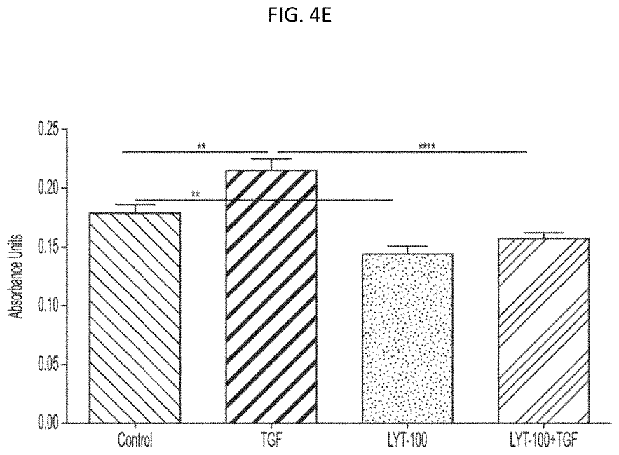

As shown at FIG. 4A, LYT-100 did not affect the survival of PMLF alone. TGF-β (5 ng/ml) significantly induced the proliferation of PMLF by nearly 45% (p=0.001), and LYT-100 did appear to diminish TGF-β-induced proliferation of PMLF by 10%, but this effect was not statistically significant (p=0.19).

### TGF-β-Induced Insoluble Collagen Synthesis Using 6-Well Plate Format

The effect of LYT-100 on inhibition of TGF-β-induced collagen synthesis was evaluated in PMLF in a 6-well format. One hundred thousand Primary Mouse Lung Fibroblasts were plated in 6-well plates and incubated in complete DMEM until they reached confluency. The incubation medium was removed and complete DMEM containing Prolin (10 μM) and Ascorbic acid (20 μg/ml) was added. LYT-100 was added to the plates at a final concentration of 500 μM 1 h prior addition of TGF-β (5 ng/ml), and cells were further incubated for 72 hrs.

Supernatant was removed, cells were washed with cold PBS, 1 ml Sircol reagent was added. The Sircol reagent contains the collagen binding dye Sirius red. The cells were scraped off with Sircol reagent and samples were shaken for 5 h at room temperature (RT), centrifuged at 10,000 rpm for 5 min, supernatant was removed, the pellet was washed in 0.5 M acetic acid to remove unbound dye, and recentrifuged at 10,000 rpm for 5 min, supernatant was removed and the final pellet was dissolved in 1 ml 0.5M NaOH and shaken at RT for 5 h. A sample of 100 μl of resultant solution was placed in 96-well. The color reaction was assessed by optical density at a wave length of 600 nm.

As shown in FIG. 4B, PMLF responded to TGF-β with increased total collagen levels, (increase of 21%; p=0.0087). LYT-100 inhibited this induction by 15% (p=0.026), as compared to the TGF-β alone, without reducing the background level of collagen.

### TGF-β-Induced Insoluble Collagen Synthesis Using 96-Well Plate Format

The effect of LYT-100 on TGF-β-induced collagen was confirmed in a high throughput collagen assay using 96-well plate format. Approximately 5,000 primary mouse fibroblasts were plated in complete DMEM in 96 well plates and incubated for 3 days at which time the cultures achieved confluency. After cells reached confluency, the medium was removed and fresh DMEM supplemented with ascorbic acid (20 μg/ml) and prolin (10 μMol) was added. LYT-100 was then added to the appropriate cultures at a final incubation concentration of 500 μM. One hour later, TGF-β was added to the appropriate cultures at a final concentration of 5 ng/ml. After 72 hours, the media was replaced with a 0.5% glutaraldehyde solution. After 30 minutes, the adherent cells were washed and subsequently incubated with acetic acid at a final concentration of 0.5M. After a 30 min room temperature incubation, and subsequent washing steps, the wells were incubated with Sircol reagent. After 5 hours, the unbound dye was removed and the plates were washed and allowed to dry. To extract collagen-bound Sircol, 100 μL of alkaline solution (0.5M NaOH) was added and plates were shaken for 1 h on rotary shaker at room temperature. Absorbance at 600 nm was determined to detect bound collagen.

As shown in FIG. 4C, in the 6-well format, TGF-β induced insoluble collagen level by 40% (p=0.0002), LYT-100 diminished this TGF-β-stimulated collagen accumulation by 24% (p=0.0003) without reducing the background level of collagen.

### TGF-β-Induced Soluble Fibronectin and Collagen Synthesis

LYT-100 was evaluated for its ability to modify TGF-β-induced soluble fibronectin and soluble collagen synthesis using a selective ELISA. Approximately 5,000 primary mouse lung fibroblasts were plated in complete DMEM in 96 well plates and incubated for 3 days at which time the cultures achieved confluency. After cells reached to confluency, medium was removed and fresh DMEM supplemented with ascorbic acid (20 μg/ml) and prolin (10 μM) was added. LYT-100 was then added to the appropriate cultures at a final incubation concentration of 500 μM. One hour later, TGF-β (5 ng/ml) was added to the appropriate cultures at a final concentration. After 72 hours, 200 μl samples of the supernatant were placed onto an ELISA plate and incubated overnight. After blocking with %1 BSA for 2 h, plates were incubated with either an anti-collagen type I antibody or an anti-fibronectin antibody.

The plates were washed after 1 hour and incubated with secondary horseradish peroxidase-conjugated antibodies (anti-goat for the collagen antibody, anti-rabbit for the fibronectin antibody). After a series of washing steps the color reagent TMB (3,3′,5,5′-Tetramethylbenzidine) was added and 15 minutes later the reactions were terminated with equal volumes of 2 N H2SO4. The levels of soluble collagen and fibronectin were determined by evaluating absorbance at 450 nm.

Referring to FIG. 4D, TGF-β induced the level of soluble fibronectin by 16% (p=0.0021). LYT-100 inhibited TGF-β-dependent induction of fibronectin by 11% (p=0.0185). Moreover, LYT-100 also inhibited the background level of soluble fibronectin by 10% (p=0.03).

As shown in FIG. 4E, TGF-β induced the level of soluble collagen by 20% (p=0.0185). LYT-100 inhibited this TGF-β-dependent increase by 36% (p=0.0001). Moreover, it also inhibited background level of soluble collagen by 23% (p=0.0115).

In summary, LYT-100 was found to: (i) reduce TGF-β-induced cell proliferation, (ii) reduce both background and TGF-β-induced levels of insoluble (structural) collagen; (iii) reduce both background and TGF-β-induced levels of soluble collagen; and (iv) reduce both background and TGF-β-induced levels of soluble fibronectin.

During the progression of IPF, an accumulation of extra cellular matrix components such as collagen and an increase in the fibroblast population is observed. Persistent proliferation of fibroblasts is considered an important contributor to the lung architecture in IPF, including the diminished interstitial spaces of the alveoli. Thus, reducing TGF-β-induced proliferation of fibroblasts and structural collagen with LYT-100 has the potential to prolong lung function in IPF. In addition to inhibiting TGF-β-induced insoluble collagen level, LYT-100 also inhibits TGF-β-induced secreted collagen and fibronectin β. Secreted collagen and fibronectin not only increase the rate of formation of fibrotic foci in the lung, these proteins can also act as ligands for integrin receptors. When integrin receptors are activated they induce not only the proliferation of epithelial cells and fibroblasts of the lungs, but they also, along with TGF-β, induce epithelial mesenchymal transition (EMT) of the epithelial cells of the lungs. EMT causes these cells to migrate to different regions of the lungs. This migration is considered to be a very important contributor for the generation of new fibrotic foci in the lungs and progression of IPF.

LYT-100 has the ability to inhibit TGF-β-induced pro-fibrotic processes and to reduce basal factors, which have the potential to exacerbate ongoing fibrosis.

### Example 5: Effect of LYT-100 on L929 Cells

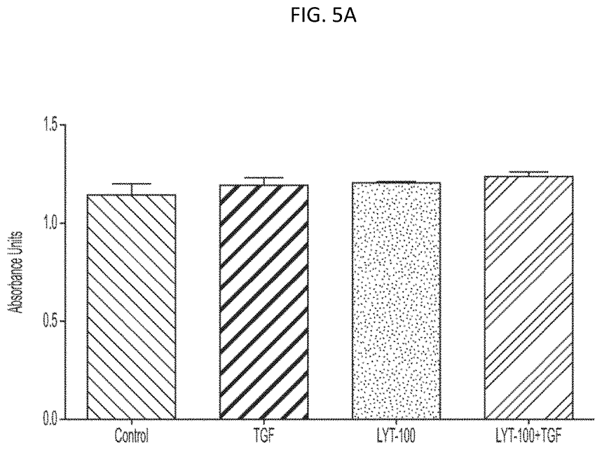

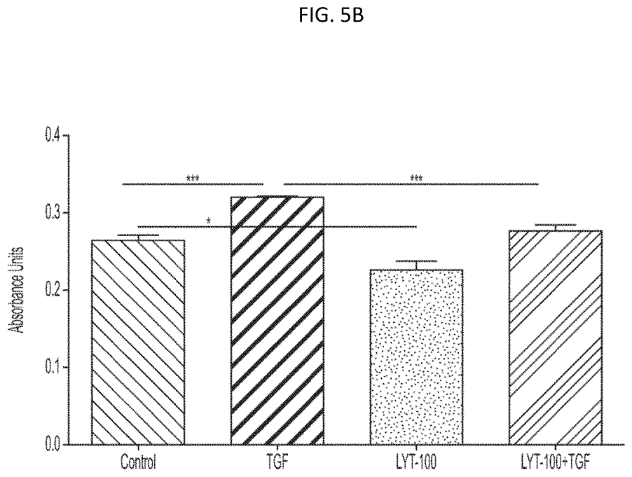

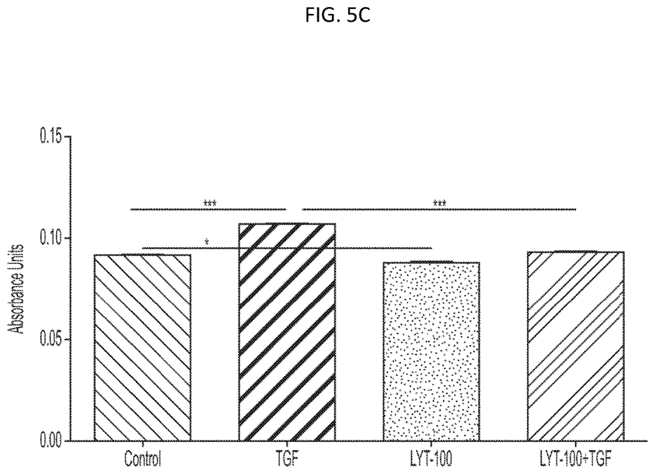

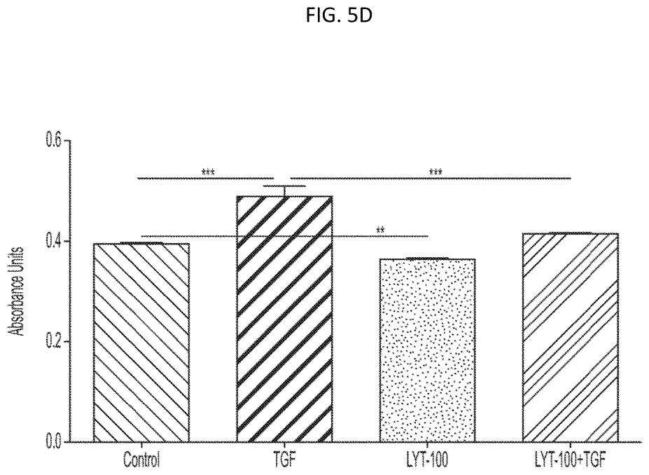

The effect of LYT-100 on survival of L929 Cells was determined. Five thousand L929 cells were plated in completed DMEN and incubated until confluency for 3 days. Medium was removed and complete DMEM containing Prolin (20 μg/ml) and ascorbic acid (10 uM) was added. LYT-100 was given at 500 μM 1 h prior addition of TGFb (5 ng/ml), and cells were further incubated for 72 hrs. 1004, of medium was removed, 204, MTT solution was added for 4 hrs, then 100 μl of DMSO was added, and absorbance of developed dark pink color was determined at 54-690 nM. FIG. 5A illustrates that LYT-100 does not affect survival of L929 cells.

The effect of LYT-100 on TGF-induced collagen synthesis in 6-wells was determined. 100,000 L929 cells were plated in complete DMEN and incubated until confluency for 3 days. Medium was removed and complete DMEM containing Prolin (20 μg/ml) and ascorbic acid (10 μM) was added. LYT-100 was given at 500 μM 1 hour prior addition of TGF-β (5 ng/ml). Cells were further incubated for 72 hrs. Supernatant was removed, cells were washed with cold PBS, 1 ml SIRCOL reagent was added onto the cells and cells were scraped off, samples were shaken for 5 h. at RT, centrifuged at 10.000 rpm for 5 min, supernatant was removed, pellet was dissolved in 0.5 M acetic acid to remove unbound dye, and re-centrifuged at 10.000 rpm for 5 min, supernatant was removed and final pelet was dissolved in 1 ml 0.5M NaOH, shaken at RT for 5 h, 100 μl of resulted solution was placed in 96-well and O.D was determined at 600. The results are summarized in FIG. 5B, which illustrates that LYT-100 inhibits TGF-induced collagen synthesis. LYT-100 also significantly inhibits collagen synthesis in the absence of added TGF-β.

Next, the effect of LYT-100 onTGF-induced collagen synthesis was confirmed using 96-well plate format. Five thousand L929 cells were plated in complete DMEN and incubated until confluency for 3 days. Medium was removed and complete DMEM containing Prolin (20 μg/ml) and ascorbic acid (10 μM) was added. LYT-100 was given at 500 μM 1 h prior addition of TGF-β (5 ng/ml). Cells were further incubated for 72 hrs. Supernatant was removed, 0.5% gluteraldehyde was added for 30 min at RT, removed, washed 3× with dd water, 0.5 M acetic acid was added for 30 min at RT, removed, washed with water, air dried and 100 μl SIRCOL dye was added for 5 h at RT. Dye was removed, plate was washed extensively under running water, air dried and 200 μl of 0.5 M NaOH was added, plates were shaken at RT for 1 h, and OD was determined at 600 nm. The results summarized in FIG. 5C illustrate that LYT-100 significantly inhibited or reduced TGF-β-induced total collagen levels. LYT-100 also significantly inhibited or reduced total collagen level in the absence of TGF-β induction.

The effect of LYT-100 on TGF-induced Soluble Collagen Synthesis was determined using a 96-well plate format. Five thousand L929 cells were plated in complete DMEN and incubated until confluency for 3 days. Medium was removed and complete DMEM containing Prolin (20 μg/ml) and ascorbic acid (10 μM) was added. LYT-100 was given at 500 μM 1 h prior addition of TGF-β (5 ng/ml). Cells were further incubated for 72 hrs. 200 μl supernatant of 96-well SIRCOL plate was placed onto ELISA plate and incubated 0/N. Next day, supernatant was removed and 100 ul of 1% BSA in PBST was added and incubated for 2 h at RT, BSA was removed, plate was washed 3× with 200 μl of PBST, and anti-collagen type I a.b was added at 1:2000 dilution (prepared in %1 BSA in PBST), incubated at RT for 1 h, primary a.b was removed, plate was washed 3× with 200 μl PBST, and secondary anti-goat HRP was added at 1:2000 dilution, incubate at RT for 1 h, removed, plate was washed 3× with 200 μl PBST and 100 μl of TMB solution was added for color development for 15 min, then 100 μl of 2 N H2SO4 was added to stop the reaction and O.D of developed yellow color was determined at 450 nm.

As illustrated in FIG. 5D, LYT-100 significantly inhibits TGF-β-induced soluble collagen levels. LYT-100 also significantly reduced soluble collagen levels in the absence of TGF-β-induction.

Fibronectin is another important component of fibrotic lungs as it is induced by TGF-β and functions both as a structural component of extra cellular matrix as well as well as a ligand for integrin receptors. Just like soluble collagen, binding of fibronectin to integrin receptors induces the proliferation of fibroblast and epithelial cells of the lungs. The effect of LYT-100 of TGF-induced soluble fibronectin synthesis was determined using a process similar to that described in the above paragraph for soluble collagen synthesis except that a fibronectin ELISA was used. As illustrated in FIG. 5E, LYT-100 significantly reduced soluble fibronectin levels, in the absence and presence of TGF-β-induction.

### Example 6: LYT-100 Study in Mouse Model of Lymphedema

This experiment tested the effect of LYT-100 in a mouse tail model of lymphedema. LYT-100 or control (carboxymethylcellose) was delivered once daily by oral gavage, in mice with ablated tail lymphatics via circumferential excision and ablation of collecting lymphatic trunks. Tail volume was measured weekly for all animals, starting pre-surgery and continuing until the occurrence of COVID19 required termination of the study at 6 weeks. At sacrifice, tails were harvested for histology and immunofluorescent imaging to characterize tissue changes with surgery and LYT-100 or control treatment. Tail volume and markers of lymphatics, fibrosis, and inflammation were compared between LYT-100 and the control group.

Animals: 14 adult (10-14 week old) C57BL/6 J mice. 7 animals per group.

Surgery: The superficial and deep collecting lymphatics of the mid portion of the tail were excised using a 2-mm full-thickness skin and subcutaneous excision performed at a distance of 15 mm from the base of the tail. Lymphatic trunks (collecting lymphatics) adjacent to the lateral veins were identified and ablated through controlled, limited cautery application under a surgical microscope.

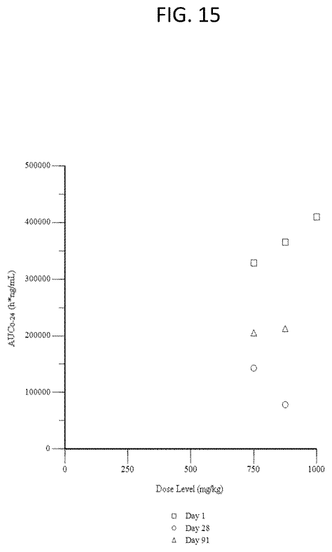

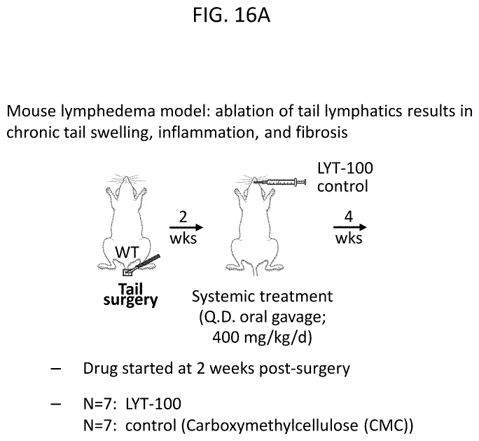

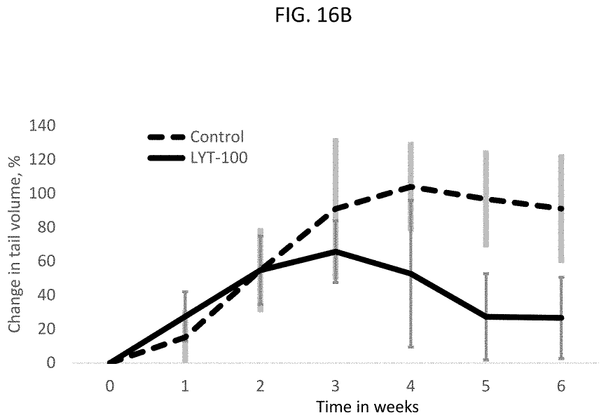

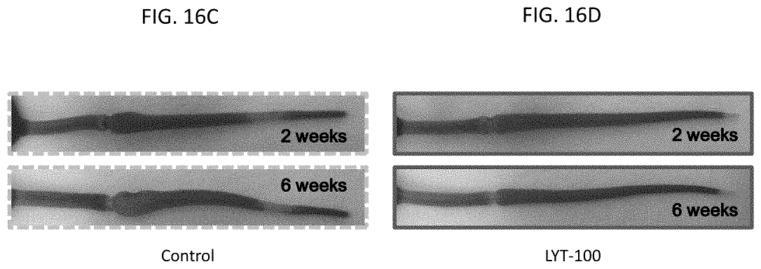

FIG. 16A-D depicts results of once daily administration of LYT-100 to reduce swelling in a mouse lymphedema model over the six weeks. The mouse lymphedema model is graphically illustrated in FIG. 16A. As shown in FIG. 16B, daily administration of LYT-100 significantly reduced swelling as compared to carboxymethylcellulose control by 5 weeks. The images in FIGS. 16C and 16D depict the differences in swelling at 6 weeks.

The dosing amounts, route and schedule are provided in Table 18.

TABLE 18

| | | | | | Dosing |
|---|---|---|---|---|---|
| | Test | | | | route and |
| Group | article | Test article preparation | Dosing | | schedule |
| Group 1 | LYT-100 | Crystals ground into fine | 250 | mg/kg/day | Oral |
| | | powder and suspended in | | | gavage, |
| | | 0.5% carboxymethycellulose | | | twice daily |
| | | (40 mg/mL) | | | |
| Group 2 | LYT-101 | Crystals ground into fine | 250 | mg/kg/day | Oral |
| | | powder and suspended in | | | gavage, |
| | | 0.5% carboxymethycellulose | | | twice daily |
| | | (40 mg/mL) | | | |
| Group 3 | Control | 0.5% carboxymethycellulose | 10 | mL/kg/day | Oral |
| | | | | | gavage, |
| | | | | | twice daily |
| Group 4 | LYT-100 | Crystals ground into fine | 250 | mg/kg/day | Oral |
| | | powder and suspended in | | | gavage, |
| | | 0.5% carboxymethycellulose | | | twice daily |
| | | (40 mg/mL) | | | |
| Group 5 | LYT-101 | Crystals ground into fine | 250 | mg/kg/day | Oral |
| | | powder and suspended in | | | gavage, |
| | | 0.5% carboxymethycellulose | | | twice daily |
| | | (40 mg/mL) | | | |
| Group 6 | Control | 0.5% carboxymethycellulose | 10 | mL/kg/day | Oral |
| | | | | | gavage, |
| | | | | | twice daily |

Measurements are provided in Table 19.

TABLE 19
Measurements

| | |
|---|---|
| Tail volume | Calculated with truncated cone formula (Sitzia 1995) and confirmed |
| | using histological measurements of soft tissue thickness of the |
| | skin/subcutaneous tissues was measured serially using digital images |
| | of histology slides stained with hematoxylin and eosin |
| Histology | Tissues fixed in 4% paraformaldehyde at 4° C., decalcified in 5% |
| | sodium EDTA (Santa Cruz Biotechnology, Dallas, Tex.), embedded |
| | in paraffin, and sectioned at 5 micrometers. Cut sections rehydrated |
| | and heat-mediated antigen unmasking performed using 90° C. sodium |
| | citrate (Sigma-Aldrich). Non-specific binding blocked with 2% |
| | BSA/20% animal serum. Tissues incubated overnight with primary |
| | antibody at 4° C. Primary antibodies used for immunohistochemical |
| | stains include goat anti-mouse LYVE-1, rat anti-mouse CD45, rabbit |
| | anti-mouse CD4, Cy3-conjugated mouse anti-αSMA (from Sigma- |
| | Aldrich), rabbit anti-human IFN-γ, rabbit anti-mouse TGF-β1, rabbit |
| | anti-mouse p-SMAD3, rabbit anti-mouse collagen I (all from |
| | ABCAM, Cambridge, MA) |
| Immunofluorescence | Immunofluorescence staining performed with AlexaFluor |
| imaging | fluorophore-conjugated secondary antibodies (Life Technologies, |
| | Norwalk, CT). Images scanned using Mirax imaging software (Carl |
| | Zeiss). Peri-lymphatic CD45+ and CD4+ cell counts assessed by |
| | counting positively stained cells within 50 μm of the most inflamed |
| | lymphatic vessel in each quadrant of the leg. Positively stained cells |
| | counted by two blinded reviewers in four randomly-selected, 40× |
| | high-power fields in a minimum of 4 fields per animal. Collagen I |
| | deposition quantified using Metamorph software (Molecular Devices, |
| | Sunnyvale, CA) in dermal areas of 5 μm cross-sections. This analysis |
| | confirmed using picrosirius red staining (Polysciences, Warrington, |
| | PA) using manufacturer's instructions. Scar index quantified with |
| | Metamorph software by calculating the ratio of red-orange:green- |
| | yellow fibers with higher numbers representing increased scarring. |

Study procedure and timing are provided in Table 20.

TABLE 20
Study Details

| | | |
|---|---|---|
| Time | Procedure | Notes |

| | | | |
|---|---|---|---|
| 0 | weeks | Surgery | Lymphatic tail |
| | | | surgery |
| 6 | weeks | Begin intervention | |
| | | (daily oral gavage) | |
| 12 | weeks | Interim sacrifice groups | |
| 18 | weeks | Late sacrifice groups | |

| | | |
|---|---|---|
| Weekly | Tail volume measurement | From pre-surgery |

| | |
|---|---|
| Statistical Analysis | ANOVA |

### Example 7: Repeat Dose Oral Toxicity and Toxicokinetics Study of LYT-100 and Pirfenidone in Sprague Dawley Rats Followed by a 4-Week Recovery Period

LYT-100 and pirfenidone were administered orally once daily for 91 consecutive days to Sprague Dawley rats to evaluate the potential reversibility of any findings following a 4-week recovery period. The profile of LYT-100, pirfenidone, and their metabolites were compared in order to understand the relationship between systemic exposure and their toxicity.

Male and female Sprague Dawley rats were separated in 6 different groups based on the 250, 500 and 750 mg/kg dose levels of LYT-100 and 750, and 875/1000 mg/kg (1000 mg/kg to male rats only on Day 1 only) dose levels of pirfenidone (Table 21).

TABLE 21
Group Assignments

| | | Dose | | | | | |
|---|---|---|---|---|---|---|---|
| | | | Dose | Dose | | Number of | |
| | | | Level | Concentration | Volume^{a} | Animals | |
| Group | Test Material | Route | (mg/kg) | (mg/mL) | (mL/kg/h) | Males | Females |
| 1 | Control | Oral | 0 | 0 | 10 | 3 | 3 |
| 2 | Deupirfenidone | Oral | 250 | 25 | 10 | 9 | 9 |
| 3 | Deupirfenidone | Oral | 500 | 50 | 10 | 9 | 9 |
| 4 | Deupirfenidone | Oral | 750 | 75 | 10 | 9 | 9 |
| 5 | Pirfenidone | Oral | 750 | 75 | 10 | 9 | 9 |
| 6 | Pirfenidone | Oral | 875 | 100^{b}/87.5 | 8.75/10^{b} | 18^{c} | 9 |

^{a}Individual dose volume was calculated based on the most recent body weight.
^{b}The first preparation of Group 6 dose formulations (prepared at 100 mg/kg) was administered at 8.75 mL/kg. The subsequent dose formulations were prepared at 87.5 mg/mL to allow for consistent dose volumes of 10 mL/kg to be administered to all study animals.
^{c}A subset of Group 6 male TK animals was administered Test Article by oral gavage once to obtain Day 1 TK profile for male animals at an adjusted dose level of 875 mg/kg.

LYT-100 (deupirfenidone, SD-560) and SD-559 (pirfenidone) were administered via an oral gavage to fed male and female Sprague Dawley rats. Dose levels were administered once daily for 91 consecutive days with 250, 500, and 750 mg/kg (LYT-100) in Groups 2, 3, and 4, respectively and 750 and 875 mg/kg (SD-559) in Groups 5 and 6, respectively. The dose level of SD-559 for Group 6 was lowered from 1000 mg/kg to 875 mg/kg on Day 1 (Tox and TK females; Tox males) and Day 2 (TK males) due to test article-related clinical signs. The original male TK animals received 1000 mg/kg of SD-559 on Day 1 and continued on study. The protocol was amended to add additional TK male animals in Group 6 for Day 1 collection of TK samples at 875 mg/kg.

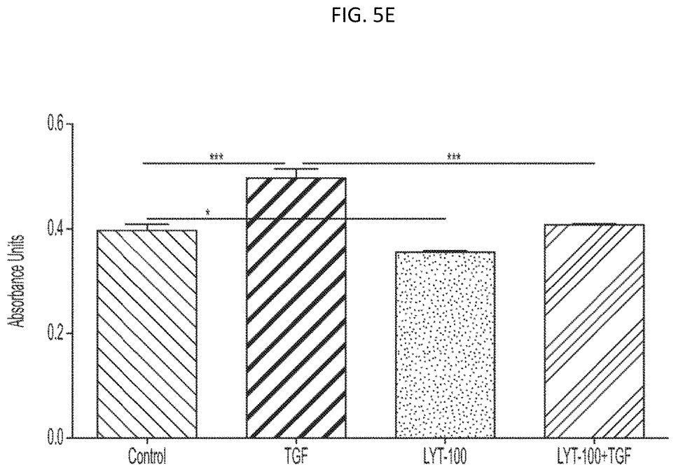

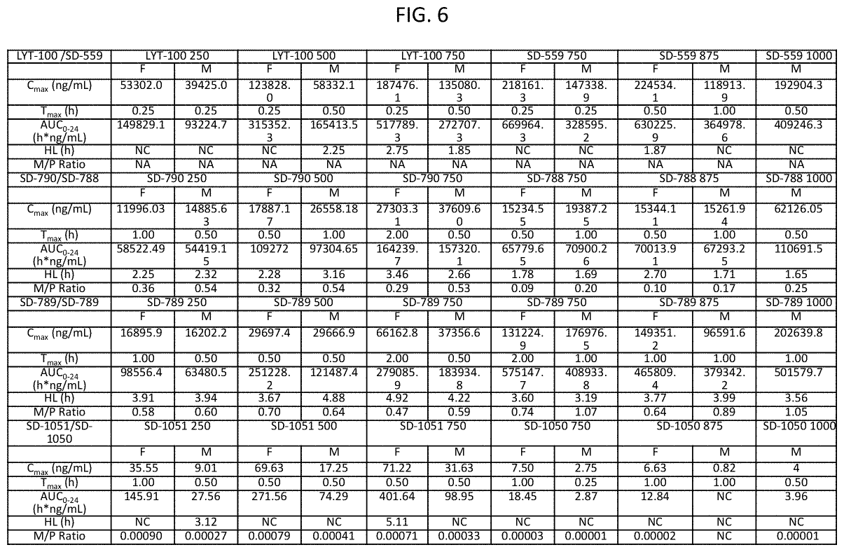

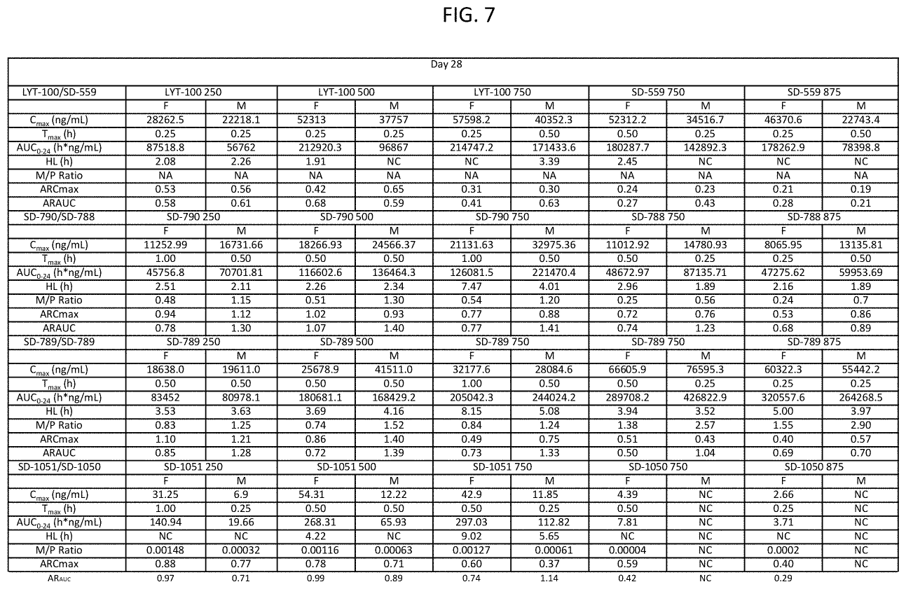

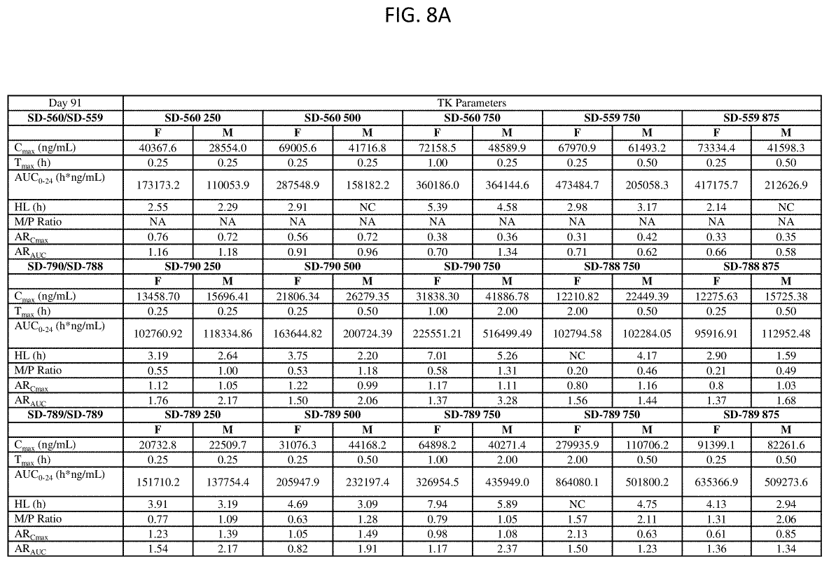

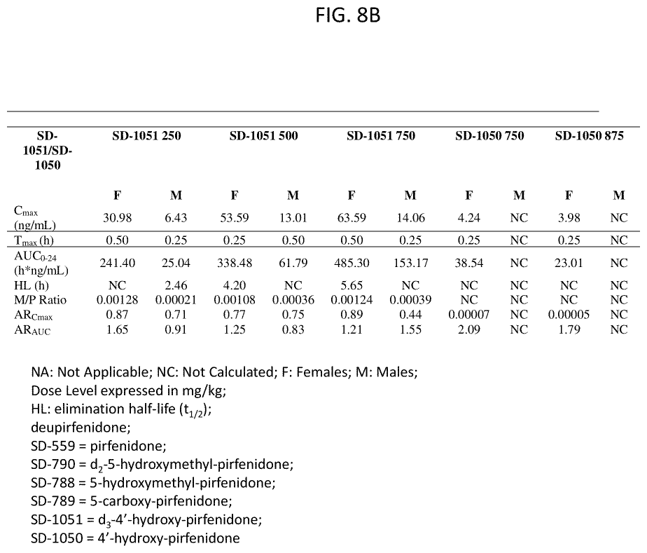

Blood samples were collected from 3 subsets of 3 animals/sex on Day 1 and Day 28 at predose, 0.25, 0.5, 1, 2, 4, 8 and 24 hours (hr) post-dose for groups 2 to 6 and from one subset of 3 animals/sex at 0.25 hr post-dose for the vehicle group (group 1). Plasma was analyzed for LYT-100 and its metabolites d2-5-hydroxymethyl-pirfenidone (SD-790; active metabolite), d3-4′-hydroxy-pirfenidone (SD-1051) and 5-carboxy-pirfenidone (SD-789; inactive metabolite) (Groups 2 to 4) and for SD-559 and its metabolites 5-hydroxymethyl-pirfenidone (SD-788; active metabolite), 4′-hydroxy-pirfenidone (SD-1050) and 5-carboxy-pirfenidone (SD-789; inactive metabolite) (Groups 5 and 6) using validated LC-MS/MS bioanalytical methods. Analysis of samples from Group 1 for all analytes confirmed showed no exposure to any compound. Non-compartmental analysis (NCA) of plasma concentration data was conducted using Phoenix® WinNonlin®, version 8.0. A summary of the toxicokinetic (TK) parameters is presented in FIGS. 6, 7, and 8A-B for Days 1, 28 and 91, respectively. An assessment of similarity in exposure for parent and metabolites between Group 4 (LYT-100 at 750 mg/kg) and Group 5 (SD-559 at 750 mg/kg) is presented in Table 22 for Day 1 and Day 28.

TABLE 22
Assessment of similarity in exposure, as assessed
by AUC, between Group 4 (LYT-100 at 750 mg/kg)
and Group 5 (SD-559 at 750 mg/kg)

| | | LYT-100/ | SD-790/ | SD-1051/ | SD-789 Group 4/ |
|---|---|---|---|---|---|
| Day | Gender | SD-559 | SD-788 | SD-1050 | SD-789 Group 5 |
| 1 | Female | 0.77 | 2.50 | 21.77 | 0.49 |
| 1 | Male | 0.83 | 2.22 | 34.51 | 0.45 |
| 28 | Female | 1.19 | 2.59 | 38.04 | 0.71 |
| 28 | Male | 1.20 | 2.54 | NC | 0.57 |

LYT-100 = deupirfenidone; SD-559 = pirfenidone; SD-790 = d2-5-hydroxymethyl-pirfenidone; SD-788 = 5-hydroxymethyl-pirfenidone; SD-789 = 5-carboxy-pirfenidone; SD-1051 = d3-4′-hydroxy-pirfenidone; SD-1050 = 4′-hydroxy-pirfenidone.

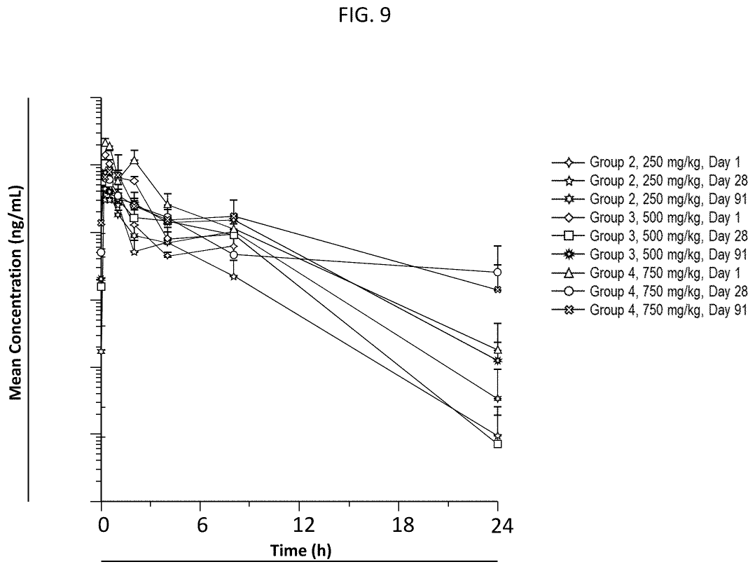

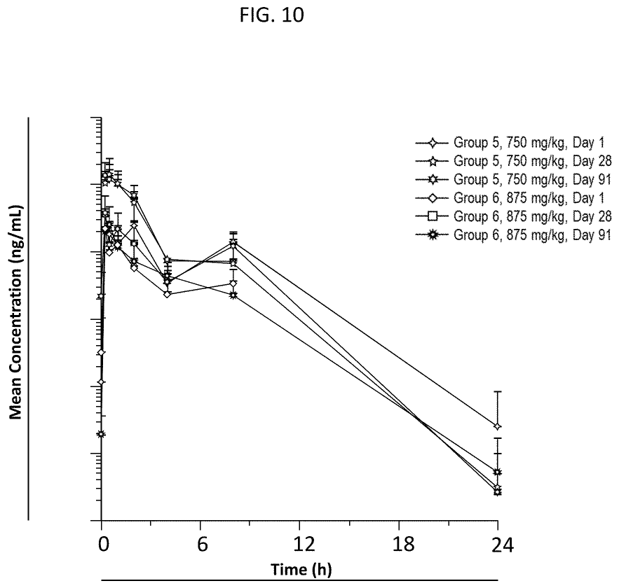

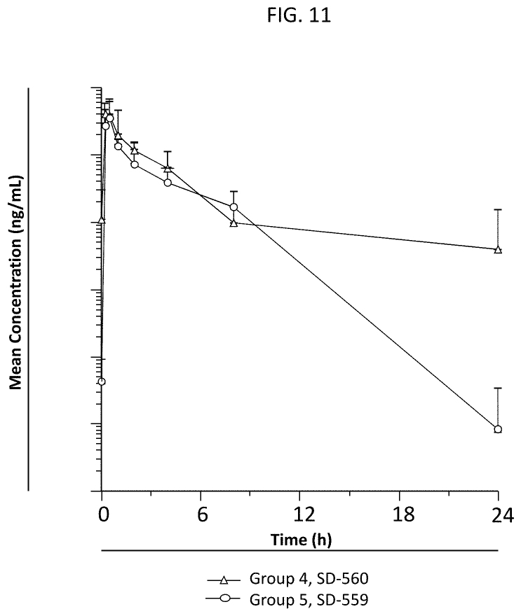

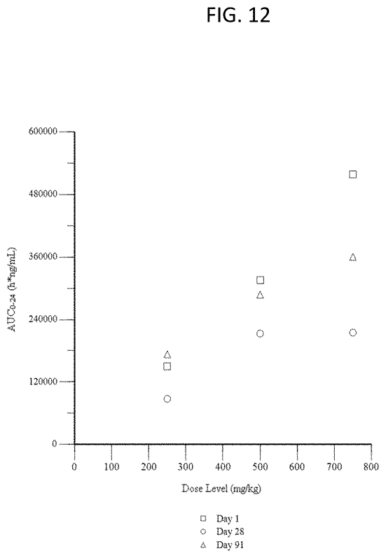

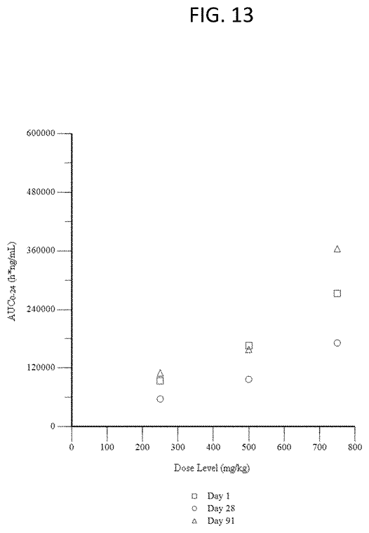

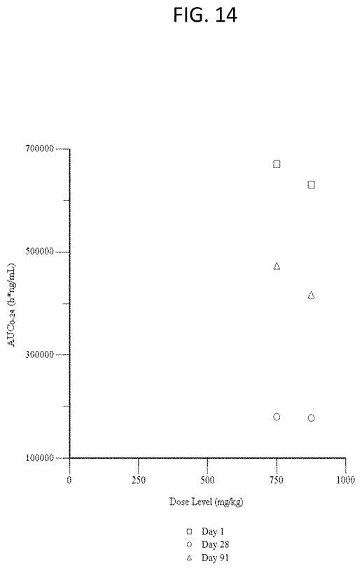

The increased exposure for LYT-100 relative to pirfenidone supports a less frequent dosing and/or lower dose than pirfenidone. Exemplary FIG. 9 and FIG. 10 depict the mean plasma concentration of LYT-100 over time following single and repeat oral administration of deupirfenidone and pirfenidone, respectively, in female Sprague Dawley rats on Days 1, 28 and 91. FIG. 11 is a chart of mean plasma concentrations following single and repeat oral administration of 750 mg/kg LYT-100 and pirfenidone in female Sprague Dawley rats on day 28. FIG. 12 and FIG. 13 are charts depicting the AUC₀₋₂₄ dose relationship following single and repeat oral administration of deupirfenidone in of female and male Sprague Dawley rats, respectively. FIG. 14 and FIG. 15 are charts depicting the AUC₀₋₂₄ dose relationship following single and repeat oral administration of pirfenidone in of female and male Sprague Dawley rats, respectively.

In animals dosed with deupirfenidone (SD-560), females generally had higher Cₘₐₓ and AUC₀₋₂₄ values than males for analytes SD-560 and SD-1051 with differences being ≥2-fold in some of the dose groups. No marked gender difference (≤2.0-fold) was observed for analytes SD-790 and SD-789 on all collection days, except Group 3 AUC₀₋₂₄ on Day 1 for SD-789 and AUC₀₋₂₄ in Group 4 on Day 91 for SD-790. Similarly, in animals dosed with pirfenidone (SD-559), females generally had higher Cₘₐₓ and AUC₀₋₂₄ values than males for analytes SD-559 and SD-1050 with differences being ≥2-fold in some of the dose groups on all collection days except for the 750 and 875 mg/kg dose levels on Days 28 and 91 due to the absence of TK parameters for males (BLQ values were observed over the complete sampling interval for all animals). No marked gender difference was observed for analytes SD-788 and SD-789 at the two dose levels on all collection days, except for SD-789 Cₘₐₓ in Group 5 on Day 91.

In animals dosed with pirfenidone (SD-559), females generally had higher Cₘₐₓ and AUC₀₋₂₄ values than males for analytes SD-559 and SD-1050 with differences being ≥2-fold in some of the dose groups on all collection days except for the 750 and 875 mg/kg dose levels on Days 28 and 91 due to the absence of TK parameters for males. No marked gender difference was observed for analytes SD-788 and SD-789 at the two dose levels on all collection days, except for SD-789 Cₘₐₓ in Group 5 on Day 91.

Exposure of SD-560 in Groups 2 to 4, as assessed with Cₘₐₓ and AUC₀₋₂₄, increased with dose where the increases were approximately less than dose proportional for Cₘₐₓ and dose proportional for AUC₀₋₂₄ over the entire dose range of 250 to 750 mg/kg. Exposure of SD-790 increased with dose where the increases were, in general, dose proportional for Cₘₐₓ and AUC₀₋₂₄ over the entire dose range of 250 to 750 mg/kg with the exception of Cₘₐₓ in females and males on Day 28 (lower than dose proportional increase) and AUC₀₋₂₄ in males on Day 91 (higher than dose proportional increase). Exposure of SD-1051 in Groups 2 through 4, as assessed with Cₘₐₓ, increased approximately in a less than dose proportional manner for Cₘₐₓ over the entire dose range, except for males on Day 1. Exposure of SD-1051, as assessed with AUC₀₋₂₄, increased dose proportionally on Day 1 over the entire dose range. However, on Days 28 and 91, the increase in AUC₀₋₂₄ was less than dose proportional in females and greater than dose proportional in males. Exposure of SD-789 in Groups 2 through 4, as assessed with Cₘₐₓ and AUC₀₋₂₄, increased with dose where the increases were generally approximately dose proportional for Cₘₐₓ (with some variability) and were dose proportional for AUC₀₋₂₄ over the entire dose range.

Exposure of SD-559, and metabolites SD-788, SD-789 and SD-1050 in Groups 5 and 6, as assessed with Cₘₐₓ and AUC₀₋₂₄ values, generally did not increase with increasing dose from 750 to 875 mg/kg and similar exposure for SD-559 and the different metabolites was observed between the two groups. The difference between dose levels is 14%, which may be masked by inter-individual variability of plasma concentrations. However, Cₘₐₓ was lower for the 875 mg/kg dose level when compared to 750 mg/kg in females on Day 91 for SD-789 and in males on Day 1 for SD-1050.

No accumulation was observed for SD-560, SD-559 or the different metabolites at the different dose levels after multiple dosing for 28 Days. Exposure to parent SD-560 or SD-559 was lower on Day 28 compared to Day 1 with accumulation ratios between 0.68 and 0.21. Lower accumulation was observed for the high dose of SD-560 and the lowest was for SD-559. Following 91 days of dosing, exposure to parent SD-560 and SD-560 was higher than on Day 28 and lower or equal to exposure on Day 1 with the lowest accumulation ratio for SD-559 (0.58-0.71). After 91 days of dosing, little to moderate accumulation was observed for SD-790 and SD-788 based AUC₀₋₂₄, for SD-789 based on AUC₀₋₂₄ following SD-560 administration and for SD-789 based on Cₘₐₓ following SD-559 administration, and SD-1050 based on AUC_{0-24.}

It is interesting to note that, while on Day 1 exposure to LYT-100 (Cₘₐₓ or AUC₀₋₂₄) was slightly lower (0.77 and 0.83 fold for females and males, respectively) than exposure of SD-559 at the same nominal dose (750 mg/kg); on Day 28 this is reversed (1.19 and 1.20 fold for females and males, respectively). This may be attributed to a slowing down of the metabolism of LYT-100 versus SD-559 because of deuterium incorporation. The stabilization imparted by deuterium substitution would manifest in a slower metabolism and therefore a higher exposure to the parent compound.

Assessment of similarity in exposure (AUC₀₋₂₄) and Cₘₐₓ between SD-560 at 750 mg/kg and SD-559 at 750 mg/kg, suggested that SD-560 showed comparable Cₘₐₓ and exposure to SD-559 on Days 1, 28 and 91 in both sexes with the exception of AUC₀₋₂₄, on day 91 for the 750 mg/kg dose. Metabolite SD-790 generated from SD-560 at 750 mg/kg showed almost twice the exposure and Cₘₐₓ on all days and in both genders vs. the non-deuterated corresponding metabolite (SD-788) generated from SD-559 at 750 mg/kg. Metabolite SD-1051, deuterated analog of SD-1050, showed significantly higher levels and exposure than metabolite SD-1050 on all days and in both genders at 750 mg/kg of SD-560 versus 750 mg/kg of SD-559. However, SD-560 sequential metabolite, SD-789, formed from SD-790 showed approximately 50% lower Cₘₐₓ and exposure than SD-789, sequential metabolite of SD-559. Increased exposure to the 5-hydroxy metabolite, SD-790 (deuterated) or SD-788 (non-deuterated), and decreased exposure to the 5-carboxy metabolite, SD-789, in animals dosed with deupirfenidone versus pirfenidone can be ascribed to deuterium stabilization against metabolism of deupirfenidone vs. pirfenidone. By slowing down the conversion of d2-5-hydroxymethyl pirfenidone (SD-790) to 5-carboxy pirfenidone (SD-789), deuterium in deupirfenidone would decrease Cₘₐₓ and exposure to 5-carboxy-pirfenidone while leading to accumulation of d2-5-hydroxy pirfenidone. Deuterium however does not appear to significantly slow down metabolism of deupirfenidone to d2-5-hydroxy pirfenidone in rat as exposure to deupirfenidone is similar to that of pirfenidone at the same dose level.

Similar trends in exposure and Cₘₐₓ of SD-560 across all dose levels (250, 500 and 750 mg/kg), after adjusting for the different doses, were observed, when compared to SD-559 at 875 mg/kg or 1000 mg/kg (male rats on Day 1).

This data also provides relevant information to human dosing. Day 1 TK data, compared to the single dose human PK data, show that the highest human exposure to parent and metabolites is covered in the present toxicity study. Exposure to LYT-100 in the TK study, as assessed by AUC, is about 5-fold larger at 750 mg/kg, on Day 1 and in male rats, than its exposure in human at the high dose of 801 mg. Exposure to deuterated 5-hydroxymethyl pirfenidone (SD-790; active metabolite) is about 1250-fold higher in the rat and exposure to 5-carboxy-pirfenidone (SD-789; inactive metabolite) in the rat is about 8-fold higher than in human, using the TK data on Day 1 in male rats for the comparison since there is a gender effect and because the exposure in male is lower than in female. Finally, the deuterated 4′-hydroxy pirfenidone metabolite (SD-1051) was not quantifiable in human. A similar conclusion can be reached when comparing Day 1 Cₘₐₓ between male rat and human, where Cₘₐₓ of LYT-100, deuterated 5-hydroxymethyl-pirfenidone (SD-790; active metabolite), and 5-carboxy-pirfenidone (SD-789; inactive metabolite) are about 15-, 1825- and 9-fold, respectively, larger in rat than in human. When using data collected in male rat after 28 days of daily dosing, the conclusion is still applicable as exposures to LYT-100, deuterated 5-hydroxymethyl-pirfenidone (SD-790; active metabolite), and 5-carboxy-pirfenidone (SD-789; inactive metabolite) are about 5-, 1600-, and 7-fold, respectively, larger in rats than in humans.

1. A method of treating lymphedema, comprising administering to a subject in need thereof an effective amount of deupirfenidone:

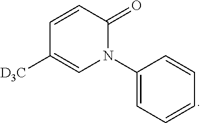 [SMILES: [2H]C([2H])([2H])c1ccc(=O)n(-c2ccccc2)c1]

2. The method of claim 1, wherein the deupirfenidone is administered orally at a total daily dose of 500 mg.

3. The method of claim 1, wherein the deupirfenidone is administered orally at a total daily dose of 1000 mg.

4. The method of claim 1, wherein the deupirfenidone is administered orally at a total daily dose of 1500 mg.

5. The method of claim 1, wherein the deupirfenidone is administered orally at a total daily dose of 2000 mg.

6-16. (canceled)

17. The method according to claim 1, wherein the subject has received treatment for cancer.

18. The method according to claim 1, wherein the subject has mild to moderate breast cancer-related lymphedema.

19. The method according to claim 1, wherein the subject is receiving or has received chemotherapy or radiation therapy.

20-26. (canceled)

27. A method of treating a fibrotic- or collagen-mediated disorder, the method comprising orally administering to a subject in need thereof the deuterium enriched pirfenidone LYT-100, wherein the administering comprises long-term dosing at a high dosage level without interruption.

28. The method according to claim 27, wherein the fibrotic- or collagen-mediated disorder is a chronic disease or disorder.

29. The method according to claim 28, wherein the chronic disease or disorder is edema.

30. The method according to claim 29, wherein the lymphedema is primary lymphedema or secondary lymphedema.

31. The method of claim 27, wherein the high dosage level is a total daily dose from about 1500 mg to about 2000 mg.

32. The method of claim 27, wherein, the long-term dosing is at least 3 months.

33. The method of claim 27, wherein the administering does not comprise up or down titration of the high dosage level during the treating.

34-42. (canceled)
